(19) United States
(12) Patent Application Publication
(10) Pub. No.: US 2018/0369186 A1
(43) Pub. Date: Dec. 27, 2018

(54) METHOD FOR MODULATING AUTOPHAGY AND APPLICATIONS THEREOF

(71) Applicant: JAWAHARLAL NEHRU CENTRE FOR ADVANCED SCIENTIFIC RESEARCH, Bangalore (IN)

(72) Inventors: Ravi MANJITHAYA, Bangalore (IN); Piyush MISHRA, Bangalore (IN); Suresh SANTHI NATESAN, Bangalore (IN); Somya BATS, Bangalore (IN); Veena AMMANATHAN, Bangalore (IN); Aravinda CHAVALMANE, Bangalore (IN)

(21) Appl. No.: 16/060,445

(22) PCT Filed: Dec. 9, 2016
(86) PCT No.: PCT/IB2016/057498

§ 371 Date: Jun. 8, 2018

(30) Foreign Application Priority Data
Dec. 9, 2015 (IN) 6596/CHE/2015

(51) Int. Cl. A61K 31/277; A61K 31/325; A61K 31/352; A61K 31/4025; A61K 31/443; A61P 25/28; A61P 25/16

(52) U.S. Cl. CPC A61K 31/277; A61K 31/325; A61K 31/352; A61P 25/16; A61K 31/443; A61P 25/28; A61K 31/4025

(57) ABSTRACT

The present disclosure relates to method of modulating autophagy by modulators of autophagy, wherein the autophagy includes but is not limited to macroautophagy, chaperone mediated autophagy and microautophagy. The present disclosure further relates to modulators of autophagy for increasing or decreasing the autophagic flux. The disclosure also relates to modulator per se in modulating autophagy including but not limiting to macroautophagy, chaperon mediated autophagy and microautophagy.

### TECHNICAL FIELD

The present disclosure relates to method of modulating autophagy by modulators of autophagy, wherein the autophagy includes but not limiting to macroautophagy, chaperone mediated autophagy and microautophagy. The present disclosure further relates to modulators of autophagy for increasing or decreasing the autophagic flux. The disclosure also relates to modulator per se in modulating autophagy.

### BACKGROUND OF THE DISCLOSURE

Autophagy is a natural degradation pathway that ensures orderly degradation of damaged or dysfunctional cellular components. It is an evolutionarily conserved process in which cell's own components are degraded by the lysosomal machinery. The process involves isolation of the targeted cytoplasmic constituents within a vesicle known as an autophagosome which is surrounded by a double-membrane. This is followed by fusion of the autophagosome with a lysosome to form an autolysosome, where the engulfed contents referred to as ‘cargo’ are subjected to enzymatic degradation. The degradation products, like amino acids and other basic building blocks, are recycled back to the cytoplasm and are used up by the cell (Rabinowitz and White 2010). This conserved pathway from yeast to humans play critical role in cell survival during nutritional deprivation, clearance of damaged/superfluous organelles, protein aggregates and intracellular pathogens. Dysfunction of autophagy leads to cell death, cancer, neurodegenerative and other diseases. Therefore, there is a need for proper function and balance in the action of the autophagy to preserve homeostasis, and present invention aims at doing so in a manner as described herein below.

### STATEMENT OF THE DISCLOSURE

Accordingly, the present disclosure relates to a method of modulating autophagy in a cell comprising step of contacting cell with at least one autophagy modulator, wherein the modulator is mTOR dependent or mTOR independent and wherein the modulator enhances autophagosome lysosome fusion or inhibits autophagosome biogenesis autophagosome maturation or degradation of autophagy proteins, degradation of autophagic cargo following authophagosome lysosome fusion.

In an embodiment, the present disclosure relates to a modulator of autophagy for enhancing formation of autolysosome by promoting autophagosome and lysosome fusion, or inhibits autophagosome biogenesis, autophagosome maturation, degradation of autophagic cargo following autophagosome-lysosome fusion, or any combination thereof, thereby decreasing autophagic flux.

In another embodiment, the present disclosure relates to a modulator of autophagy, wherein the modulator enhances formation of autolysosome by promoting fusion of autophagosome and lysosome, thereby increasing autophagic flux or inhibits at least one of autophagosome biogenesis, autophagosome maturation, degradation of autophagic cargo inside vacuole after autophagosome-lysosome fusion, or any combination thereof, thereby decreasing autophagic flux.

### BRIEF DESCRIPTION OF THE ACCOMPANYING FIGURES

In order that the invention may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

FIG. 1 illustrates development of dual luciferase assay to monitor autophagy in real time, wherein

(A) illustrates the degradation of luciferase assay, wherein S. cerevisiae shuttle vectors pRS306 (URA) and pRS305 (LEU) are used to clone the POT1 promoter and the firefly and Renilla luciferase genes, respectively.

(B) illustrates gradual decrease in luciferase counts upon induction of autophagy in wild type cells, whereas cells carrying core autophagy mutants atg1 and atg5 and selective autophagy mutant atg36 (adaptor protein for pexophagy) did not show any drop in the luciferase activity over time.

FIG. 2 illustrates screening of small molecule libraries, wherein

(A) illustrates screening two small molecule libraries for their effect on autophagy using luciferase based assay for monitoring autophagy.

(B) illustrates dose dependent effect on the rates of degradation of firefly luciferase by Bay11-7082 and ZPCK.

FIG. 3 illustrates the effect of 6-Bio on autophagy, wherein

(A) illustrates box plot (representative plot for 100 compounds) demonstrating hits from small molecule library of pharmacologically active compounds, LOPAC¹²⁸⁰, screened in S. cerevisiae toxicity model of α-synuclein. In the box plot, compounds that rescued the growth lag due to α-synuclein toxicity (denoted by absorbance, A₆₀₀) of WT α-synuclein-EGFP strains≥3 SD units (grey box) are considered hits (blue) and the ones that did not rescue the growth lag due to α-synuclein toxicity are in green. WT EGFP (black) and untreated WT α-synuclein-EGFP (red) represent the positive and negative controls.

(B) illustrates growth curve of WT EGFP cells with or without 6-Bio (50 μM) treatment.

(C) illustrates western blot of GFP-Atg8 processing assay under growth condition, wherein fusion protein GFP-Atg8 accumulation and free GFP release is monitored across time course (0 h and 6 h) with or without 6-Bio (50 μM) treatment, respectively.

(D) illustrates western blot of GFP-Atg8 processing assay under starvation condition, wherein fusion protein GFP-Atg8 accumulation and free GFP release is monitored across time course (0 h, 2 h, 4 h and 6 h) with or without 6-Bio (50 μM) treatment, respectively.

(E) illustrates microscopy images of ptf LC3 transfected HeLa cells treated with 6-Bio (5 μm) and quantification of autophagosome and autolysosome indicating fold change over its untreated counterpart (n=25), scale bar=15 μm.

FIG. 4 illustrates effect of 6-Bio on α-synuclein in an autophagy dependant manner, wherein

(A) Illustrates microscopy images of WT α-synuclein-EGFP yeast cells treated with or without 6-Bio (50 μM) for 16 h, vacuole stained with CMAC-Blue (100 nM), scale bar=2 μm.

(B) illustrates quantification plot for α-synuclein-EGFP degradation assay in wild-type (WT) yeast strain under growth condition upon treatment with 6-Bio (50 μM).

(C) illustrates quantification plot for α-synuclein-EGFP degradation assay in wild-type (WT) yeast strain under starvation condition upon treatment with 6-Bio (50 μM).

(D) illustrates quantification plot for α-synuclein-EGFP degradation assay in autophagy mutant (atg1Δ) strain under growth condition upon treatment with 6-Bio (50 μM).

(E) illustrates quantification plot for α-synuclein-EGFP degradation assay in autophagy mutant (atg1Δ) strain under starvation condition upon treatment with 6-Bio (50 μM).

(F) illustrates western blot (below) and graph (above) indicating fold change in EGFP-α-synuclein degradation in SH-SY5Y cells upon treatment with 6-Bio (5 μM), 3-MA (5 mM) and both, respectively.

FIG. 5 illustrates enhancement of mTOR dependant autophagy by 6-Bio and confers neuroprotection in a mouse MPTP toxicity model, wherein

(A) illustrates western blots indicating dose-dependent modulation of autophagy related proteins (LC3, P70S6 kinase and 4E-BPI) by 6-Bio in HeLa cells.

(B) Illustrates photomicrographs of TH⁺ immunostained dopaminergic neurons in SNpc (arrow) of mouse midbrain in Control group, MPTP treated (23 mg/kg body weight) mouse, 6-Bio (5 mg/kg body weight) treated mouse and both [Prophylaxis (MPTP+Pro) and Co-administration (MPTP+Co)], scale bar=300 μm.

(C) illustrates stereological quantification indicating the number of TH⁺ DA and its intensity in SNpc neurons.

(D)illustrates densitometric quantification indicating the number of TH⁺ DA and its intensity in SNpc neurons.

FIG. 6 illustrates growth curve (A), growth rate (B) and doubling time (C) of WT α-synuclein-EGFP (red curve) versus WT EGFP (black curve) in S. cerevisiae α-synuclein toxicity model.

FIG. 7 illustrates schematic representation of small molecule library screened in S. cerevisiae α-synuclein toxicity model.

FIG. 8 illustrates effect of 6-Bio in autophagy mutants expressing α-synuclein, wherein

(A) illustrates a plot indicating the percent growth of WT α-synuclein-EGFP strain in presence of Agk2 (50 μM) and 6-Bio (50 μM).

(B) illustrates growth of autophagy mutants (atg1Δ, atg5Δ, atg8Δ, atg11Δ and atg15Δ) expressing α-synuclein-EGFP observed with or without 6-Bio (50 μM).

FIG. 9 illustrates α-synuclein-EGFP degradation assays in yeast, wherein

(A) illustrates a schematic representation of α-synuclein-EGFP degradation assay conditions.

(B) illustrates western blots for α-synuclein-EGFP degradation upon 6-Bio (50 μM) administration in wild type cell under growth condition.

(B) illustrates western blots for α-synuclein-EGFP degradation upon 6-Bio (50 μM) administration in wild type cell under starvation condition.

(C) illustrates western blots for α-synuclein-EGFP degradation upon 6-Bio (50 μM) administration in autophagy mutant (atg1Δ) cells under growth condition.

(D) illustrates western blots for α-synuclein-EGFP degradation upon 6-Bio (50 μM) administration in autophagy mutant (atg1Δ) cells under starvation condition.

FIG. 10 illustrates the effect of 6-Bio administration in MPTP mouse model, wherein

(A) illustrates the schedule of dosage administration of MPTP (23 mg/kg) and 6-Bio (5 mg/kg) in mice groups.

(B) illustrates photomicrographs of TH⁺ immunostained DA neurons in SNpc of mouse midbrain of control, MPTP, 6-Bio and both [Prophylaxis (MPTP+Pro) and Co-administration (MPTP+Co)]groups.

(C) illustrates quantitative plot of SNpc volume of mouse brains for all the groups (control, MPTP, 6-Bio and both [Prophylaxis (MPTP+Pro) and Co-administration (MPTP+Co)]).

FIG. 11 illustrates Pot1 GFP assay for Acacetin under nitrogen starvation medium.

FIG. 12 illustrates fold change in colony forming units for Acacetin using Burden assay, wherein

(A) illustrates the effect of acacetin on U1752 cells infected with Salmonella typhimurium SL1344.

(B) illustrates the effect of acacetin on HeLa cells infected with Salmonella typhimurium SL1344,

FIG. 13 illustrates growth Curve of Salmonella typhimurium SL1344 post incubation with acacetin, gentamycin and combination of acacetin and gentamycin, respectively.

FIG. 14 illustrates co-localization GFP-LC3 with mcherry in HeLa cells infected with Salmonella typhimurium SL1344, followed by treatment with gentamycin and acacetin.

FIG. 15 illustrates live cell microscopic images of GFP-LC3 transfected HeLa cells infected with mcherry-Salmonella typhimuriumSL1344, wherein

(A) illustrates the imageslive cell microscopic images of GFP-LC.3 transfected HeLa cells infected with mcherry-Salmonella typhimuriumSL1344 and treated with gentamycin.

(B) illustrates the imageslive cell microscopic images of GFP-LC3 transfected HeLa cells infected with mcherry-Salmonella typhimuriumSL1344 and treated with gentamycin, followed by acacetin.

(C) illustrates intensity of the red channel featured in live cell microscopic images measured using image J-Stacks T function.

FIG. 16 illustrates Traffic Light Assay for Acacetin, wherein

(A) illustrates ptf-LC3 transfected HeLa cells treated with Acacetin for 2 hours.

(B) illustrates number of autophagosomes and autolysosomes counted using image J-cell counter function.

FIG. 17 illustrates the effect of Bay11-7082 and ZPCK on autophagy, wherein

(A) illustrates POT1-GFP processing assay for accessing the effect of Bay11-7082 and ZPCK pexophagy under starvation condition.

(B) illustrates GFP-Atg8 assay for accessing the effect of Bay11-7082 and ZPCK on general autophagy.

(C) illustrates pexophagy as monitored via fluorescence microscopy.

(D) illustrates protease protection assay depicting conversion of precursor to matured form of aminopeptidase on treatment with proteinase K in Bay11-7082 treated cells.

FIG. 18 illustrates inhibitory effect of Bay11-7082 and ZPCK on autophagy in HeLa cells.

FIG. 19 illustrates induction of autophagy in mice brain by 6-Bio to clear toxic protein aggregates. (A) Representative immuno histofluorescent photomicrographs of various cohorts namely control, MPTP (23 mg/kg of body weight), 6-Bio (5 mg/kg of body weight) and MPTP+Co that were stained for LC3B (an autophagy marker) and TH (SNpc) in midbrain. Autophagic modulation by 6-Bio were evaluated in DAergic neurons in SNpc and the LC3B puncta fold change per neuron was quantitated (B). (C) Representative immuno histofluorescent photomicrographs of above mentioned cohorts were stained for A11 (toxic oligomers) and TH (SNpc) midbrain. Aggregate clearance by 6-Bio were evaluated in DAergic neurons in SNpc and the A1 puncta fold change per neuron was quantitated (D). Statistical analysis was performed using one-way ANOVA and post-hoc Bonferroni test. Scale bar=50 μm. Error bars, mean±SEM, ns-non significant, ***-P<0.00.1.

FIG. 20 illustrates amelioration of MPTP-induced behavioral deficits by 6-Bio. Effect of 6-Bio (5 mg/kg) on (A) latency to fall of various cohorts namely Placebo, MPTP and MPTP+Co as assessed by rotarod test (B) Representative trajectory maps of all mentioned cohorts as analyzed by open field test. (C) Periphery distance travelled by all indicated cohorts as assessed by open field test. Effect of 6-Bio (5 mg/kg) on various cohorts namely Placebo, MPTP and MPTP+Post. (D) latency to fall of various cohorts namely Placebo, MPTP and MPTP+Post as assessed by rotarod test. (E) Periphery distance travelled by all indicated cohorts as assessed by open field test. Both rotarod and open field behavior analyses performed on day 13 or day 7 post-MPTP/vehicle administrations. Both rotarod and open field behavior analyses performed on day 13 or day 7 post-MPTP/vehicle administrations. 6-Bio (5 mg/kg) was administrated either along with MPTP (MPTP+Co) or post 48 h of MPTP administration (MPTP+Post). Statistical analysis was performed using one-way ANOVA and post-hoc Bonferroni test. Scale bar=50 μm. Error bars, mean±SEM. ns-non significant, ***-P<0.001.

FIG. 21 illustrates blockage of initial step of autophagy by Bay-11 whereas ZPCK acts towards the later stages of autophagy in yeast Saccharomyces cerevisiae. (A) Pot01-GFP processing assay for assessing the effect of Bay11 and (B) ZPCK on pexophagy. No free GFP release was seen on treatment of wild type cells with Bay11 even after 6 hours of starvation, whereas very little free GFP was observed only at the later time points in ZPCK treated cells as quantified in (C) and (D). Effect of Bay11 (E) and ZPCK (F) on general autophagy was monitored by GFP18 Atg8 assay. No or delayed release of GFP was observed on treatment with either Bay11 or ZPCK respectively as compared to the untreated cells (G). Pexophagy (degradation of peroxisomes via autophagy) as monitored via fluorescence microscopy revealed that Bay11 acted at a step prior to fusion of autophagosomes with the vacuole (H) (labelled with FM4-64). No free GFP was seen inside the vacuole and the peroxisomes were present in the cytosol even on autophagy induction, morphology similar to an early step mutant of autophagy Δatg1 (H), (I) and (J). On treatment with ZPCK, peroxisomes got accumulated inside the vacuole, morphology similar to Δatg15, an autophagy mutant deficient in the vacuolar proteases (H), (I) and (J). (K) Quantitation showing percentage number of cells with diffused GFP accumulation inside the vacuole in different treatment conditions after 6 hours in starvation. (L) Graph showing percentage number of cells with accumulation of pexophagic bodies inside the vacuole on starvation in wild type, Δatg15 and wild type cells treated with ZPCK.

FIG. 22 illustrates effect of Bay11 treatment on maturation of autophagosomes. (A) GFP-Atg8 fluorescence microscopy showed an accumulation of GFP-Atg8 positive puncta on treatment with Bay11 under starvation condition. Graphs showing diffused GFP inside the vacuole (B) and number of puncta in the cytosol at 4 hours of starvation (C) in wild type, Δypt7 and wild type cells treated with Bay11. (D) To elucidate the step of action of Bay11, a protease protection assay was performed using aminopeptidase as a marker, which is also a substrate for autophagy on starvation. Conversion of precursor to matured form of aminopeptidase on treatment with proteinase K (PK) in Bay11 treated cells indicated that the cargo is not protected by the autophagosome. (E) Quantitation showing relative precursor and mature form of aminopeptidase levels for different treatment groups. Y-axis shows the total aminopeptidase levels. TX-Triton X-100; PK-Proteinase K. (F) Co-localization of genomically tagged GFP-Atg8 and Atg5-RedStar* proteins in untreated and Bay11 treated conditions. (G) Quantitation showing percentage number of cells with more than one Atg5 puncta. (H) Quantitation showing number of co-localization events per 100 cells in untreated and Bay11 treated cells. Scale bar=5 μm. Data shown represent a minimum of 100 cells from 3 independent experiments and are expressed as the mean±SD. ***P<0.001 (individual means compared using two-tailed unpaired t-test).

FIG. 23 illustrates inhibition of autophagy by Bay11 and ZPCK in MEFs. (A and B) MEFs were treated with DMSO (vehicle control), 5 μm Bay11 or 5 μM ZPCK for 24 h or 48 h, fixed for immunofluorescence analysis with anti-p62 antibody and imaged by confocal microscopy (A). Analysis was done for the percentage of cells with accumulated endogenous p62+aggregates (B). Scale bar, 20 μm. (C and D) Atg5+/+ (wild-type) and Atg5−/− (autophagy6 deficient) MEFs were treated with DMSO (vehicle control) or 5 μM Bay11 for 24 h, followed by immunoblotting analysis with anti-p62 and anti-GAPDH antibodies. Densitometric analysis of p62 levels was done relative to GAPDH where the control (DMSO9 treated) condition was fixed at 100%. (E) Atg5+/+ and Atg5−/− MEFs were treated with DMSO (vehicle control) or 5 μM Bay11 for 24 h, followed by immunoblotting analysis with anti-MAP1LC3B and anti-GAPDH antibodies. (F) MAP1LC3B-II/MAP1LC3B-1 and (G) MAP11 LC3B-II/GAPDH levels quantitated for 3 independent experiments in DMSO and Bay11 treated cells.

FIG. 24 illustrates inhibition of autophagy by Bay 11 and ZPCK in HeLa cells at different stages. (A) Hela cells transfected with ptf-MAP1LC3B (vector having tandem mRFP-GFP tagged MAP1LC3B) treated with either Bay11 or ZPCK for 2 hours in growth medium in the presence or absence of Bafilomycin A1 (400 nM) were observed under fluorescence microscope. Autophagosomes appear as yellow dots whereas autolysosomes appear red inside the cells. On treatment with ZPCK, autolysosomes increased inside the cells whereas on Bay11 treatment, very few autophagosomes were seen. Scale bar=15 μM (B and C) Data shown represent a minimum of 65 cells from 3 independent experiments with number of autophagosomes and autolysosomes counted and are expressed as the mean SD. ***P<0.001 (One way ANOVA, individual means compared with a Dunnett's Multiple Comparison Test) (D) MAP1LC3B conversion assay for the mentioned treatment groups under nutrient rich, starvation conditions and along with bafilomycin A1, (E-G) MAP1LC3B2 II/MAP1LC3B-1 and MAP1LC3B-II/TUBB levels were quantified for all conditions and plotted. (H) MAP1LC3B conversion assay in the absence and presence of Bay11 for 2 and 12 hours. (I) MAP1LC3B-II/MAP1LC3B-1 and (J) MAP1LC3B-II/TUBB levels of control and Bay11 treated cells over a time course for 3 independent experiments. (K) immunostaining with p62 antibody in RFP-MAP1LC3B transfected HeLa cells with co-localization. Scale bar=20 μM (L) Graph showing the amount of Co-localization between p62 and RFP8 MAP1LC3B in different treatment groups. The mean intensity of colocalized dots was calculated using Co-localization plug-in of Image J analysis software. (M) EGFR trafficking shown by immunoblot for the mentioned treatment groups and degradation levels quantified (N). Data shown represent a minimum of 65 cells from 3 independent experiments and are expressed as the mean±SD. ***, p<0.001; **, p<0.01; *, p<0.05; ns, non-significant (individual means compared by two-tailed unpaired t-test).

FIG. 25 illustrates effect of autophagy modulators in lace plant (Aponogeton madagascariensis) cells. Lace plant leaves treated with different modulators were sectioned and stained using monodansyicadaverine (MDC) and scanned via confocal microscopy with 405/450±35 nm (ex/em). (A) There were significantly more punctate structures (autophagosome like structures) in overnight starvation treatment compared to the control. Scale bar=20 μm (B) The 1 μM concanamycin A and 5 μM rapamycin had a significantly higher number of puncta compared to control, which had more than the 5 μM wortmannin treatment. (C) 50 μM Bay11 significantly reduced, whereas 50 μM ZPCK increased puncta compared to the control. (D) Quantitation of the mean number of punctate structures for each treatment (E) Treatment with 50 μM Bay11 or 5 μM wortmannin of overnight starvation leaves showed fewer puncta as compared to the control. Punctate structures were significantly higher than the control in the Starvation, and 50 μM ZPCK treatments. Data shown represent a minimum of 4 independent experiments and are expressed as the mean±SEM. (One way ANOVA, Dunnett's multiple comparison test (***, p<0.001; **, p<0.01; *, p<0.05). Scale bar 30 μm.

FIG. 26 illustrates immunolocalization of Atg8 in lace plant (Aponogeton madagascariensis) cells. Lace plant leaf pieces treated with modulators revealed similar results to the MDC staining. (A) The starvation, 5 μM rapamycin and 1 μM concanamycin A treatment groups contained more puncta than the control, while the 5 μM wortmannin treatment reduced puncta. (B) 50 μM Bay11 reduced the number of puncta and 50 μM ZPCK increased puncta compared to the control group. (C) Quantitation was done for a minimum of 4 independent replicates per experimental group and statistical significance was calculated (One way ANOVA, Dunnett's multiple comparison test (***, p<0.001; **, p<0.01. *, p<0.05). Scale bar: 30 μm.

FIG. 27 illustrates decrease in intracellular Salmonella typhimurium by Acacetin.

FIG. 28 illustrates decrease in intracellular Salmonella typhimurium by Acacetin.

FIG. 29 illustrates decrease in intracellular Salmonella typhimurium by Acacetin.

FIG. 30 illustrates that Acacetin does not have direct anti-bacterial effect.

FIG. 31 illustrates that Acacetin increases temporal recruitment of LC3 to mcherry Salmonella typhimurium.

FIG. 32 illustrates recruitment of p62 to mcherry Salmonella typhimurium.

FIG. 33 illustrates increased temporal recruitment of p62 to mcherry Salmonella typhimurium by Acacetin.

FIG. 34 illustrates live cell imaging of Acacetin treated cells.

FIG. 35 illustrates arrest of replication of Salmonella in presence of Acacetin.

FIG. 36 illustrates non-functionality of Acacetin in Atg5 KO HeLa cell line.

FIG. 37 illustrates non-functionality of Compound G in presence of autophagy inhibitors.

FIG. 38 illustrates results of burden assay for screening of compounds.

FIG. 39 illustrates XCT 790 is a potent autophagy inducer and protects α-synuclein toxicity by clearing them in autophagy dependent manner in yeast. (a) Representative box plot indicating chemical hits attained from small molecule library screened in α-synuclein toxicity model of S. cerevisiae. In the box plot, small molecules that rescued the growth (absorbance, A600) of wild-type (WT) α-synuclein-EGFP strain by SD units (grey box) are considered as hits (blue) and that do not rescue the growth are labeled in green. WT EGFP (black) and untreated WT α-synuclein-EGFP (red) strains represent the positive and negative controls of the screen. (b) Percent growth of yeast strains (WT EGFP, WT α-syn-EGFP, atg1Δ EGFP, atg1Δ α-syn-EGFP) treated with XCT 790 (n=4, three independent experiments). (c) Representative western blot of GFP Atg8 processing assay and assessed the GFP-Atg8 processing after 6h of incubation in growth condition treated with XCT 790, Fold change in autophagy induction (EGFP-Atg8 band intensity normalized by loading control) and its flux (summation of EGFP-Atg8 and free EGFP normalized by loading control) modulated by XCT 790 were quantified (three independent experiments), PGK1 served as a loading control. (d) Microscopy images of WT α-synuclein-EGFP treated with XCT 790 and then quantified for the vacuolar free EGFP in fold (n=75 cells). Scale bar 5 μm. (e) Representative western blot for α-synuclein-EGFP degradation in WT α-synuclein-EGFP strain analyzed after 24 h of XCT 790 treatment and quantified for the levels of α-synuclein-EGFP (three independent experiments). Gapdh served as a loading control. (f) Representative western blot for α-synuclein-EGFP degradation in atg1Δ α-synuclein-EGFP strain analyzed after 24 h of XCT 790 treatment and quantified for the levels of α-synuclein-EGFP (three independent experiments). Gapdh served as a loading control. Concentration of XCT 790 used was 50 μM. Statistical analysis was performed using one-way ANOVA and post-hoc Bonferroni test. Error bars, mean±SEM. ns-non significant, ***-P<0.001.

Example 40 illustrates exertion of cellular neuroprotection by XCT 790 in an autophagy dependent mechanism. (a) Representative western blot of LC3 processing assay in SHSY-5Y cells treated with XCT 790 (2 h) under growth condition and normalized LC3-II levels were quantified. β-tubulin was used as a loading control. (b) Representative microscopy images of tandem RFP-EGFP-LC3 assay in HeLa cells treated with XCT 790 for 2 h. Yellow puncta was autophagosomes and red was autolysosomes. Fold change in autophagosomes and autolysosomes by XCT 790 were quantified. Scale bar was 15 μm. (c) Graph indicating the cell viability read out of SHSY-5Y overexpressing EGFP-α-synuclein treated with XCT 790 in presence of pharmacological autophagy inhibitor 3-MA. Cell viability was analysed using CellTitre Glo (Promega) assay. More RLU readout was indicative of more cell viability and vice-versa. (d) Representative western blots of mTOR substrates like P70S6K (phospho and total form) and 4EBP1 (phospho and total form) regulation by various treatments like XCT 790, EBSS and LiCl. β-tubulin was used as a loading control. (e) Representative western blots of signaling pathway proteins like AMPK (phospho and total form) and ULK1 (phospho and total form) regulation by XCT 790 and EBSS. β-tubulin was used as a loading control. Concentrations of XCT 790, 3-MA and LiCl used were 5 μM, 100 nM and 10 mM. Statistical analysis was performed. using one-way ANOVA and post-hoc Bonferroni test, Error bars, mean±SEM. ns-non significant, **-P<0.01, ***-P<0.001.

FIG. 41 illustrates modulation of autophagy by XCT 790 through ERRα, (a) ERRα protein levels after transfecting either scrambled siRNA (100 picomoles) or ERRα siRNA (100 picomoles) for 48 h in HeLa cells was analyzed by western blotting and then quantified. β-tubulin was used as a loading control. (b and c) Microscopy images (b) of tandem RFP-EGFP-LC3 assay in XCT 790 treated HeLa cells (2h) post ERRα siRNA transfection (48 h). Cells were immunostained for ERRα in various treatments. Scale bar was 15 μm. Quantification (c) of autophagosomes (Yellow puncta) and autolysosomes (red puncta) modulated by XCT 790 treatment in. ERRα siRNA transfected cells. (d and e) Microscopy images (d) of tandem RFP-EGFP-LC3 assay in XCT 790 treated HeLa cells (2 h) post ERRα Flag transfection (48 h). Cells were immunostained for ERRα in all treatment groups. Scale bar used was 15 μm. Quantification (e) of autophagosomes (Yellow puncta) and autolysosomes (red puncta) modulated by XCT 790 treatment in ERRα Flag transfected cells. Concentration of XCT 790 used was 5 μM. Statistical analysis was performed using one-way ANOVA and post-hoc Bonferroni test. Error bars, mean=SEM. ns-non significant, **-P<0.01, ***-P<0.001.

FIG. 42 illustrates localization of ERRα onto autophagosomes to modulate autophagy. (a) Microscopy images of tandem RFP-EGFP-LC3 assay in HeLa cells transfected (48 h) with either ERRα siRNA or ERRα Flag treated with XCT 790 for 2 h. Cells were immunostained for ERRα. Scale bar was 15 μm. (b) PCC (Pearson's Colocalization Coefficient) analyses of ERRα with either autophagosome (yellow) or autolysosomes(red) in HeLa cells transfected (48 h) with either ERRα siRNA or ERRα Flag treated with XCT 790 for 2 h were plotted. Statistical analysis was performed using one-way ANOVA and post-hoc Bonferroni test. Error bars, mean±SEM. ns-non significant, *-P<0.05, ***-P<0.001.

FIG. 43 illustrates neuroprotective effect of XCT 790 by degrading toxic protein aggregates through inducing autophagy in DAergic neurons of midbrain of mice. (a) Representative photomicrographs of whole brain and SNpc for various cohorts namely vehicle, MPTP (23 mg/kg of body weight) and MPTP+Co (Co-administration of MPTP and XCT 790: MPTP; 2 mg/kg of body weight and XCT 790; 5 mg/kg of body weight). Scale bar is 600 μm. (b) Graph representing the unbiased stereological quantification of TH-ir DA neurons in SNpc for above mentioned cohorts. (c) RepresentativeIHC photomicrographs of SNpc DAergic neurons double stained for A11 (toxic oligomer marker) and TH (SNpc marker) antibodies for the above mentioned cohorts. Scale bar is 50 μm. (d) Plot indicating the A11 puncta per DAergic neuron in SNpc was quantitated for all cohorts. (e) Representative fluorescent IHC photomicrographs of DAergic neurons in SNpc double stained for LC3 (autophagy marker) and TH (SNpc marker) antibodies for various cohorts namely vehicle, MPTP XCT only and MPTP+Co. Scale bar is 50 μm. (f) Graph representing the LC3 puncta per neuron for various cohorts.

FIG. 44 illustrates amelioration of MPTP-induced behavioural impairments by XCT 790. Latency to fall for various cohorts such as vehicle, MPTP and MPTP+Co on both day 13 (a) and 15 (b) were monitored using rotarod test. (c) Representative trajectory maps were indicated for all the mentioned cohorts. (d and e) Plots indicating the peripheral distance travelled by mice were assessed through open field test on both day 13 (d) and 15 (e).

FIG. 45 illustrates non-toxicity of XCT 790 to yeast (a) Growth curve and its related parameters like growth rate (b) and doubling time (c) of XCT 790 treated WT EGFP. Growth rate and doubling time plots of XCT 790 treated WT α-syn-EGFP (d and e) and atg1Δ α-syn-EGFP (f and g) cells.

FIG. 46 illustrates modulation of starvation-induced autophagy by XCT 790 in yeast. (a) Representative blot for GFP-Atg8 processing of XCT 790 treated yeast cells monitored across time points under starvation condition (2, 4 and 6 h). Modulation of autophagy induction (total EGFP/PGK1) and autophagy flux (free EGFP/PGK2) upon XCT 790 treatment, were quantified and then plotted. PGK1 served as a loading control. (b) Scheme illustrating the protocol followed for u-synuclein degradation assay in yeast. Statistical analysis was performed using one-way ANOVA and post-hoc Bonferroni test. Error bars, mean±SEM. ns-non significant, *-P<0.05, ***-P<0.001.

FIG. 47 illustration non-toxicity of XCT 790 to cells (Hela and SHSY5Y) and scheme for α-synculein toxicity assay. Cell viability of cell lines like HeLa (a) and SH-SY5Y (b) after 72 h of XCT 790 treatments for various indicated concentrations. Cell viability was assayed using CellTitre Glo (Promega) kit.

FIG. 48 illustrates modulation of autophagy by XCT 790 in mTOR-independent manner in SH-SY5Y cells. (a) Representative microscopy images of tandem RFP-EGFP-LC3 assay in SH-SY5Y cells treated with XCT 790 for 2 h. Yellow puncta was autophagosomes and red was autolysosomes. Fold change in autophagosomes and autolysosomes by XCT 790 were quantified and plotted. Scale bar was 15 μm. (b) Representative western blots of mTOR substrates like P70S6K (phospho and total form) and 4EBP1 (phospho and total form) regulation by various treatments like XCT 790, EBBS and LiCl in SH-SY5Y cells. β-tubulin was used as a loading control. Statistical analysis was performed using one-way ANOVA and post-hoc Bonferroni test. Error bars, mean±SEM. ns-non significant, *-P<0.05, **-P<0.01.

FIG. 49 illustrates that autophagic function of XCT 790 is unaffected in presence of actinomycin D. (a) Representative microscopy images of tandem RFP-EGFP-LC3 assay in HeLa cells co-treated with XCT 790 and actinomycin D (act D). Scale bar 15 μm. (b) Fold change of autophagosomes and autolysosomes across various treatments were plotted. Statistical analysis was performed using one-way ANOVA and post-hoc Bonferroni test. Error bars, mean±SEM. ns-non significant, ***-P<0.001.

FIG. 50 illustrates administration of XCT 790 in mice MPTP toxicity model. (a) Dosage regimen of XCT 790 in various cohorts namely vehicle, MPTP (23 mg/kg of body weight) and MPTP+Co (MPTP; 23 mg/kg of body weight and XCT 790; 5 mg/kg of body weight). (b) Plot indicating the densitometric quantification (B), measure of TH intensity in DAergic neurons. (c) Plot indicating the nigral volume was measured for the cohorts. Statistical analysis was performed using one-way ANOVA and post-hoc Bonferroni test. Error bars, mean±SEM. ***-P<0.001.

FIG. 51 illustrates scheme for the behavior study. Scheme indicating the dosage regimen of various cohorts such as vehicle (a), MPTP (b) and MPTP+Co (c) followed for the behavioral study.

### DETAILED DESCRIPTION OF DISCLOSURE

The present disclosure relates to a method of modulating autophagy in a cell comprising step of contacting cell with at least one autophagy modulator, wherein the modulator is mTOR dependent or mTOR independent and wherein the modulator enhances autophagosome lysosome fusion or inhibits autophagosome biogenesis autophagosome maturation or degradation of autophagy proteins, degradation of autophagic cargo following authophagosome lysosome fusion

In an embodiment, the modulator of autophagy is selected from a group comprising (2′Z,3′E)-6-Bromoindirubin-3′-oxime (6-Bio), acacetin, 7-dihydroxy-2-(4-methoxyphenyl)chromen-4-oneN6-(4-Aminobenzyl)-9-[5-(methylcarbonyl)-β-D-ribofuranosyl]adenine(AB-MECA, Lapidine; (3S,3aR,4R,8aR)-3-hydroxy-6,8a-dimethyl-8-oxo-3-propan-2-yl-2,3a,4,5-tetrahydro-1H-azulen-4-yl] (E)-2-methylbut-2-enoate, Senecionine, 12-Hydroxysenecionan-11,16-dione, XCT790; (3-[4-(2,4Bis-trifluorormethylbenzyloxy)-3-methoxyphenyl]-2-cyano-N-(5-trifluoromethyl-1,3,4-thiadiazol-2-yl)acrylamide), PD180970 (6-(2,6-Dichlorophenyl)-2-[(4fluoro-3-methylphenyl)amino]-8-methyl-pyrido[2,3-d]pyrimidin-7(8H)-one), Ritodrine hydrochloride; (N-(p-Hydroxyphenethyl)-4-hydroxynorephedrine hydrochloride) and SB 242084 dihydrochloride hydrate; (6-Chloro-2,3-dihydro-5-methyl-N-[6-[(2-methyl-3-pyridinyl)oxy]-3-pyridinyl]-1H-indole-1-carboxyamide dihydrochloride hydrate), N-Carbobenzyloxy-L-phenylalanylchloromethyl ketone (ZPCK), 3-[(4-methylphenyl)sulfonyl]-(2E)-propenenitrile (Bay11-7082), Elaidylphosphocholine, N-[4-(1H-Benzimidazol-2-yl)phenyl]-5-nitro-2-thiophenecarboxamide, Ethyl [(2-{[(5-nitro-2-thienyl)carbonyl]amino}-3-thienyl)carbonyl]carbamate, 2-Phenyl-N-[5-(3-thienyl)-1,3,4-oxadiazol-2-yl]-2H-1,2,3-triazole-4-carboxamide, 3-Bromo-N-[5-(5,6-dihydro-1,4-dioxin-2-yl)-1,3,4-oxadiazol-2-yl]benzamide, N-(6-Chloro-1,3-benzothiazol-2-yl)-2-(4-fluorophenyl)-N-(3-pyridinylmethyl)acetamide. N-(2-Methoxybenzyl)-5-nitro-4,6-pyrimidinediamine, N-[5-(2,5-Dichloro-3-thienyl)-1,3,4oxadiazol-2-yl]-5-nitro-2-furamide, N-2,5-Dimethylphenyl)-2-{[3-(3-methoxyphenyl)-4-oxo-3,4,6,7-tetrahydrothieno[3,2-d]pyrimidin-2-yl]sulfanyl}acetamide, 4-Fluoro-N-[2-(4-fluorophenyl)-2-(4-methyl-1-piperazinyl)ethyl]-3-methylbenzenesulfonamide, N-[5-(3,4-Dimethylphenyl)-1,3,4-oxadiazol-2-yl]-5-nitro-2-furamide, N-[3-(1,3-Benzothiazol-2-yl)-6-methyl-4,5,6,7-tetrahydrothieno[2,3-c]pyridin-2-yl]-4-cyanobenzamide hydrochloride (1:1), N-(4,5-Diphenyl-1,3-thiazol-2-yl)-1,2-oxazole-5-carboxamide, 2-[5-(2,4-Difluorophenyl)-1,2-oxazol-3-yl]-N-(1,2-oxazol-3-yl)acetamide, 1-(2,4-Dihydroxy-3-methylphenyl)-2-4-propylphenoxy)ethanone, N-[5-(2,4-Dimethylphenyl)-1,3,4-oxadiazol-2-yl]-3,5-dimethoxybenzamide, N-[3-(1,3-Benzothiazol-2-yl)-6-ethyl-4,5,6,7-tetrahydrothieno[2,3-c]pyridin-2-yl]-1-methyl-1H-pyrazole-3-carboxamide hydrochloride (1:1), 2-(2-Bromobenzyl)-1,2,3,4-tetrahydro-3-isoquinolinecarboxamide, 2-[(5,7-Dibromo-8-quinolinyl)oxy]propanamide, 3-Amino-N-[4-(imidazo[1,2-a]pyridin-2-yl)phenyl]-2-pyrazinecarboxamide, 1-Benzyl-N-[5-chloro-2-(1-piperidinyl)phenyl]-1H-1,2,3-triazole-4-carboxamide, (4-Bromophenyl){4[(5-chloro-2-thienyl)sulfonyl]-1-piperazinyl}methanone, 1-Phenyl-N-(1,3,4-thiadiazol-2-yl)-3-(2-thienyl)-1H-pyrazole-5-carboxamide, 5-{[(7-Bromo-2,3-dihydro-1,4-benzodioxin-6-yl)methyl]sulfanyl}-N-cyclopropyl-1,3,4-thiadiazol-2-amine, 1-Ethyl-7-methyl-4-oxo-1,4-dihydro-1,8-naphthyridine-3-carboxylic acid, Pentachlorophenol, N,N,N-Trimethyl-1-hexadecanaminium bromide, 1,4-Dihydroxy-5,8-bis({2-[(2-hydroxyethyl)amino]ethyl}amino)-9,10-anthraquinone dihydrochloride, (9E)-9-Octadecen-1-yl 2-(trimethylammonio)ethyl phosphate, 4-(1H-Indazol-3-yl)-N-(4-piperidinyl)-1H-pyrrolo[2,3-b]pyridin-6-amine, 2-(4-Methoxybenzyl)-6-(4,5,6,7-tetrahydropyrazolo[1,5-a]pyrazin-2-ylmethyl)-6,7-dihydro-5H-pyrrolo[3,4-d]pyrimidine, [(E)-octadec-9-enyl] 2-(trimethylazaniumyl)ethyl phosphate), TNP (N6-[(4-nitrophenyl)methyl]-N2-[[3-(trifluoromethyl)phenyl]methyl]-9H-Purine-2,6-diamine), Cinchonidine; (R)-[(2S,4S,5R)-5-ethenyl-1-azabicyclo[2,2,2]octan-2-yl]-quinolin-4-ylmethanol, Trioxsalen; (2,5,9-Trimethylfuro[3,2-g]benzopyran-7-one) or any combination thereof.

In another embodiment, the modulator of autophagy is 6-Bio, XCT-790, ZPCK., acacetin or Bay-11.

In yet another embodiment, 6-Bio or XCT-790 enhances autolysosome formation in the cell and causes degradation of α-synuclein (SNCA).

In still another embodiment, the 6-Bio enhances fusion of autophagosome and lysosome in the cell by about 8 fold to 10 fold.

In still another embodiment, the 6-Bio modulates autophagy by passive diffusion and the 6-Bio is mTOR dependent and GSK3B dependent.

In still another embodiment, the XCT-790 is mTOR independent and ERRα dependent.

In still another embodiment, the Bay-11 inhibits autophagosome lysosome fusion, autophagosome biogenesis or autophagosome maturation.

In still another embodiment, the ZPCK inhibits degradation of autophagic cargo inside the vacuole after fusion of autophagosome and lysosome.

In still another embodiment, the acacetin induces formation of autophagolysosome in the cell infected with intracellular microorganism.

In still another embodiment, the intracellular microorganism is selected from a group comprising Salmonella typhimurium Legionella pneumophila, Listeria monooytogenes, Shigella flexneri, Streptococcus pyrogenes, Mycobacterium tuberculosis, or any combination thereof.

In still another embodiment, the autophagy is selected from a group comprising macroautophagy, chaperone mediated autophagy, microautophagy, mitophagy, pexophagy, liphophagy, reticulophagy, ribophagy, zymophagy, Aggrephagy, xenophagy, or any combinations thereof.

In still another embodiment, the cell is eukaryotic cell selected from a group comprising yeast cell, plant cell and mammalian cell, or a combination thereof.

In still another embodiment, the concentration of the modulator is ranging from about 1 μM to about 150 μM.

The present disclosure further relates to a modulator of autophagy for enhancing formation of autolysosome by promoting autophagosome and lysosome fusion, or inhibits autophagosome biogenesis, autophagosome maturation, degradation of autophagic cargo following autophagosome-lysosome fusion, or any combination thereof, thereby decreasing autophagic flux.

In an embodiment, the modulator of autophagy is selected from a group comprising (2′Z,3′E)-6-Bromoindirubin-3′-oxime (6-Bio), acacetin, 7-dihydroxy-2-(4-methoxyphenyl)chromen-4-oneN6-(4-Aminobenzyl)-9-[5-(methylcarbonyl)-β-D-ribofuranosyl]adenine(AB-MECA, Lapidine; (3S,3aR,4R,8aR)-3-hydroxy-6,8a-dimethyl-8-oxo-3-propan-2-yl-2,3a,4,5-tetrahydro-1H-azulen-4-yl](E)-2-methylbut-2-enoate, Senecionine, 12-Hydroxysenecionan-11,16-dione, XCT790; (3-[4-(2,4-Bis-trifluoromethylbenzyloxy)-3-methoxyphenyl]-2-cyano-N-(5-trifluoromethyl-1,3,4-thiadiazol-2-yl)acrylamide), PD180970 (6-(2,6-Dichlorophenyl)-2-[(4fluoro-3-methylphenyl)amino]-8-methyl-pyrido[2,3-d]pyrimidin-7(8H)-one), Ritodrine hydrochloride; (N-(p-Hydroxyphenethyl)-4-hydroxynorephedrine hydrochloride) and SB 242084 dihydrochloride hydrate; (6-Chloro-2,3-dihydro-5-methyl-N-[6-[(2-methyl-3-pyridinyl)oxy]-3-pyridinyl]-1H-indole-1-carboxyamide dihydrochloride hydrate), N-Carbobenzyloxy-L-phenylalanylchloromethyl ketone (ZPCK), 3-[(4-methylphenyl)sulfonyl]-(2E)-propenenitrile (Bay11-7082), Elaidylphosphocholine, N-[4-(1H-1-Benzimidazol-2-yl)phenyl]-5-nitro-2-thiophenecarboxamide, Ethyl [(2-{[(5-nitro-2-thienyl)carbonyl]amino}-3-thienyl)carbonyl]carbamate, 2-Phenyl-N-[5-(3-thienyl)-1,3,4-oxadiazol-2-yl]-2H-1,2,3-triazole-4-carboxamide, 3-Bromo-N-[5-(5,6-dihydro-1,4-dioxin-2-yl)-1,3,4-oxadiazol-2-yl]benzamide, N-(6-Chloro-1,3-benzothiazol-2-yl)-2-(4-fluorophenyl)-N-(3-pyridinylmethyl)acetamide, N-(2-Methoxybenzyl)-5-nitro-4,6-pyrimidinediamine, N-[5-(2,5-Dichloro-3-thienyl)-1,3,4-oxadiazol-2-yl]-5-nitro-2-furamide, N-(2,5-Dimethylphenyl)-2-{[3-(3-methoxyphenyl)-4-oxo-3,4,6,7-tetrahydrothieno[3,2-d]pyrimidin-2-yl]sulfanyl}acetamide, 4-Fluoro-N-[2-(4-fluorophenyl)-2-(4-methyl-1-piperazinyl)ethyl]-3-methylbenzenesulfonamide, N-[5-(3,4-Dimethylphenyl)-1,3,4-oxadiazol-2-yl]-5-nitro-2-furamide, N-[3-(1,3-Benzothiazol-2-yl)-6-methyl-4,5,6,7-tetrahydrothieno[2,3-c]pyridin-2-yl]-4-cyanobenzamide hydrochloride (1:1), N-(4,5-Diphenyl-1,3-thiazol-2-yl)-1,2-oxazole-5-carboxamide, 2-[5-(2,4-Difluorophenyl)-1,2-oxazol-3-yl]-N-(1,2-oxazol-3-yl)acetamide, 1-(2,4-Dihydroxy-3-methylphenyl)-2-(4-propylphenoxy)ethanone, N-[5-(2,4-Dimethylphenyl)-1,3,4-oxadiazol-2-yl]-3,5-dimethoxybenzamide, N-[3-(1,3-Benzothiazol-2-yl)-6-ethyl-4,5,6,7-tetrahydrothieno[2,3-c]pyridin-2-yl]-1-methyl-1H-pyrazole-3-carboxamide hydrochloride (1:1), 2-(2-Bromobenzyl)-1,2,3,4-tetrahydro-3-isoquinolinecarboxamide, 2-[(5,7-Dibromo-8-quinolinyl)oxy]propanamide, 3-Amino-N-[4-(imidazo[1,2-a]pyridin-2-yl)phenyl]-2-pyrazinecarboxamide, 1-Benzyl-N-[5-chloro-2-(1-piperidinyl)phenyl]-1H-1,2,3-triazole-4-carboxamide, (4-Bromophenyl){4-[(5-chloro-2-thienyl)sulfonyl]-1-piperazinyl}methanone, 1-Phenyl-N-(1,3,4-thiadiazol-2-yl)-3-(2-thienyl)-1H-pyrazole-5-carboxamide, 5-{[(7-Bromo-2,3-dihydro-1,4-benzodioxin-6-yl)methyl]sulfanyl}-N-cyclopropyl-1,3,4-thiadiazol-2-amine, 1-Ethyl-7-methyl-4-oxo-1,4-dihydro-1,8-napthyridine-3-carboxylic acid, Pentachlorophenol, N,N,N-Trimethyl-1-hexadecanaminium bromide, 1,4-Dihydroxy-5,8-bis({2-[(2-hydroxyethyl)amino]ethyl}amino)-9,10-anthraquinone dihydrochloride, (9E)-9-Octadecen-1-yl 2-(trimethylammonio)ethyl phosphate, 4-(1H-Indazol-3-yl)-N-(4-piperidinyl)-1H-pyrrolo[2,3-b]pyridin-6-amine, 2-(4-Methoxybenzyl)-6-(4,5,6,7-tetrahydropyrazolo[1,5-a]pyrazin-2-ylmethyl)-6,7-dihydro-5H-pyrrolo [3,4-d] pyrimidine, [(E)-octadec-9-enyl] 2-(trimethylazaniumyl)ethyl phosphate), TNP (N6-[(4-nitrophenyl)methyl]-N2-[[3-(trifluoromethyl)phenyl]methyl]-9H-Purine-2,6-diamine), Cinchonidine; (R)-[(2S,4S,5R)-5-ethenyl-1-azabicyclo[2.2.2]octan-2-yl]-quinolin-4-ylmethanol, Trioxsalen; (2,5,9-Trimethylfuro[3,2-g]benzopyran-7-one) or any combination thereof.

In another embodiment, the 6-Bio or XCT-790 enhances autolysosome formation in the cell and causes degradation of ax-synuclein (SNCA).

In yet another embodiment, the 6-Bio modulates autophagy by passive diffusion and wherein the 6-Bio is mTOR dependent and GSK3B dependent; wherein the XCT-790 is mTOR independent and ERRα dependent while modulating the autophagy and wherein the XCT-790 is inverse agonist of ERRα.

The present disclosure further relates to a modulator of autophagy, wherein the modulator enhances formation of autolysosome by promoting fusion of autophagosome and lysosome, thereby increasing autophagic flux or inhibits at least one of autophagosome biogenesis, autophagosome maturation, degradation of autophagic cargo inside vacuole after autophagosome-lysosome fusion, or any combination thereof, thereby decreasing autophagic flux.

In an embodiment, the modulator is selected from a group comprising (2′Z,3′E)-6-Bromoindirubin-3′-oxime (6-Bio), acacetin, 7-dihydroxy-2-(4-methoxyphenyl)chromen-4-oneN6-(4-Aminobenzyl)-9-[5-(methylcarbonyl)-β-D-ribofuranosyl]adenine(AB-MECA, Lapidine; (3S,3aR,4R,8aR)-3-hydroxy-6,8a-dimethyl-8-oxo-3-propan-2-yl-2,3a,4,5-tetrahydro-1H-azulen-4-yl](E)-2-methylbut-2-enoate, Senecionine, 12-Hydroxysenecionan-11,16-dione, XCT790; (3-[4-(2,4-Bis-trifluoromethylbenzyloxy)-3-methoxyphenyl]-2-cyano-N-(5-trifluoromethyl-1,3,4-thiadiazol-2-yl)acrylamide), PD180970 (6-(2,6-Dichlorophenyl)-2-[(4-fluoro-3-methylphenyl)amino]-8-methyl-pyrido[2,3-d]pyrimidin-7(8H)-one), Ritodrine hydrochloride; (N-(p-Hydroxyphenethyl)-4-hydroxynorephedrine hydrochloride) and SB 242084 dihydrochloride hydrate; (6-Chloro-2,3-dihydro-5-methyl-N-[6-[(2-methyl-3-pyridinyl)oxy]-3-pyridinyl]-1H-indole-1-carboxyamide dihydrochloride hydrate), N-Carbobenzyloxy-L-phenylalanylchloromethyl ketone (ZPCK), 3-[(4-methylphenyl)sulfonyl]-2E)-propenenitrile (Bay11-7082),Elaidylphosphocholine, N-[4-(1H-Benzimidazol-2-yl)phenyl]-5-nitro-2-thiophenecarboxamide, Ethyl [(2-{[(5-nitro-2-thienyl)carbonyl]amino}-3-thienyl)carbonyl]carbamate, 2-Phenyl-N-[5-(3-thienyl)-1,3,4-oxadiazol-2-yl]-2H-1,2,3-triazole-4-carboxamide, 3-Bromo-N-[5-(5,6-dihydro-1,3,4-oxadiazol-2-yl]benzamide, N-(6-Chloro-1,3-benzothiazol-2-yl)-2-(4-fluorophenyl)-N-(3-pyridinylmethyl)acetamide, N-(2-Methoxybenzyl)-5-nitro-4,6-pyrimidinediamine, N-[5-(2,5-Dichloro-3-thienyl)-1,3,4-oxadiazol-2-yl]-5-nitro-2-furamide, N-(2,5-Dimethylphenyl)-2-{[3-(3-methoxyphenyl)-4-oxo-3,4,6,7-tetrahydrothieno[3,2-d]pyrimidin-2-yl]sulfanyl}acetamide, 4-Fluoro-N-[2-(4-fluorophenyl)-2-4-methyl-1-piperazinyl)ethyl]-3-methylbenzenesulfonamide, N-[5-(3,4-Dimethylphenyl)-1,3,4-oxadiazol-2-yl]-5-nitro-2-furamide, N-[3-(1,3-Benzothiazol-2-yl)-6-methyl-4,5,6,7-tetrahydrothieno[2,3-c]pyridin-2-yl]-4-cyanobenzamide hydrochloride (1.1), N-(4,5-Diphenyl-1,3-thiazol-2-yl)-1,2-oxazole-5-carboxamide, 2-[5-(2,4,-Difluorophenyl)-1,2-oxazol-3-yl]-N-(1,2-oxazol-3-yl)acetamide, 1-(2,4-Dihydroxy-3-methylphenyl)-2-(4-propylphenoxy)ethanone, N-[5-(2,4-Dimethylphenyl)-1,3,4-oxadiazol-2-yl]-3,5-dimethoxybenzamide, N-[3-(1,3-Benzothiazol-2-yl)-6-ethyl-4,5,6,7-tetrahydrothieno[2,3-c]pyridin-2-yl]-1-methyl-1H-pyrazole-3-carboxamide hydrochloride (1:1), 2-(2-Bromobenzyl)-1,2,3,4-tetrahydro-3-isoquinolinecarboxamide, 2-[(5,7-Dibromo-8-quinolinyl)oxy]propanamide, 3-Amino-N-[4-(imidazo[1,2-a]pyridin-2-yl)phenyl]-2-pyrazinecarboxamide, 1-Benzyl-N-[5-chloro-2(1-piperidinyl)phenyl]-1H-1,2,3-triazole-4-carboxamide, (4-Bromophenyl){4-[(5-chloro-2-thienyl)sulfonyl]-1-piperazinyl}methanone, 1-Phenyl-N-(1,3,4-thiadiazol-2-yl)-3-2-thienyl)-1H-pyrazole-5-carboxamide, 5-{[(7-Bromo-2,3-dihydro-1,4-benzodioxin-6-yl)methyl]sulfanyl}-N-cyclopropyl-1,3,4-thiadiazol-2-amine, 1-Ethyl-7-methyl-4-oxo-1,4-dihydro-1,8-naphthyridine-3-carboxylic acid, Pentachlorophenol, N,N,N-Trimethyl-1-hexadecanaminium bromide, 1,4-Dihydroxy-5,8-bis({2-[(2-hydroxyethyl)amino]ethyl}amino)-9,10-anthraquinone dihydrochloride, (9E)-9-Octadecen-1-yl 2-(trimethylammonio)ethyl phosphate, 4-(1H-Indazol-3-yl)-N-(4-piperidinyl)-1H-pyrrolo[2,3-b]pyridin-6-amine, 2-(4-Methoxybenzyl)-6-(4,5,6,7-tetrahydropyrazolo[1,5-a]pyrazin-2-ylmethyl)-6,7-dihydro-5H-pyrrolo[3,4-d]pyrimidine, [(E)-octadec-9-enyl] 2-(trimethylazaniumyl)ethyl phosphate), TNP (N6-[(4-nitrophenyl)methyl]-N2-[[3-trifluoromethyl)phenyl]methyl]-9H-Purine-2,6-diamine), Cinchonidine; (R)-[(2S,4S,5R)-5-ethenyl-1-azabicyclo[2.2.2]octan-2-yl]-quinolin-4-ylmethanol, Trioxsalen; (2,5,9-Trimethylfuro[3,2-g]benzopyran-7-one) or any combination thereof.

In another embodiment, the 6-Bio or XCT-790 enhances autolysosome formation in the cell and causes degradation of +-synuclein (SNCA); wherein the 6-Bio modulates autophagy by passive diffusion and wherein the 6-Bio is mTOR dependent and GSK3B dependent; and wherein the XCT-790 is mTOR independent and ERRα dependent while modulating the autophagy and wherein the XCT-790 is inverse agonist of ERRα.

The present disclosure relates to a method for modulating autophagy.

In an embodiment of the present disclosure, the method of modulating autophagy involves treating cells with modulators of autophagy.

In an embodiment, the modulator of autophagy is mTOR dependent or mTOR independent, wherein the modulator in the method enhances formation of autolysosome by promoting fusion of autophagosome and lysosome.

In another embodiment, the modulator of autophagy employed in the method is mTOR dependent and GSK3B dependent.

In another embodiment, the modulator of autophagy employed in the method is mTOR independent but ERRα dependent while modulating the autophagy.

In an embodiment, the method of the present disclosure modulates autophagy including but not limiting to macroautophagy, chaperone mediated autophagy and microautophagy

In an embodiment, in the method of the present disclosure the modulator includes but not limited to activator, wherein the modulator which is an activator enhances formation of autolysosomes by promoting fusion of autophagosome and lysosome, thereby increasing autophagic flux.

In an embodiment, the method of modulating autophagy comprises the step of treating cells with modulator of autophagy which is mTOR dependent or mTOR independent, wherein the modulator enhances formation of autolysosome by promoting fusion of autophagosome and lysosome, thereby increasing autophagic flux.

In an embodiment, the modulator which is an activator of autophagy is selected from a group comprising (2′Z,3′E)-6-Bromoindirubin-3′-oxime (6-Bio), acacetin 5,7-dihydroxy-2-(4 methoxyphenyl)chromen-4-one N6-(4-Aminobenzyl)-9-[5-(methylcarbonyl)-β-D-ribofuranosyl]adenine(AB-MECA), Lapidine; [(3S,3aR,4R,8aR)-3-hydroxy-6,8a-dimethyl-8-oxo-3-propan-2-yl-2,3a,4,5-tetrahydro-1H-azulen-4-yl](E)-2-methylbut-2-enoate, Senecionine; 12-Hydroxysenecionan-11,16-dione, XCT790; (3-[4(2,4-Bis-trifluoromethylbenzyloxy)-3-methoxyphenyl]-2-cyano-N-(5-trifluoromethyl-1,3,4-thiadiazol-2-yl)acrylamide), PD180970; (6-(2,6-Dichlorophenyl)-2-[(4-fluoro-3-methylphenyl)amino]-8-methyl-pyrido[2,3-d]pyrimidin-7(8H)-one), Ritodrine hydrochloride; (N-(p-Hydroxyphenethyl)-4-hydroxynorephedrine hydrochloride) and SB 242084 dihydrochloride hydrate; (6-Chloro-2,3-dihydro-5-methyl-N-[6-[(2-methyl-3-pyridinyl)oxy]-3-pyridinyl]-1H-indole-1-carboxyamide dihydrochloride hydrate), or any combination thereof.

In an exemplary embodiment, the modulator which is an activator of macroautophagy, an activator of chaperone mediated autophagy and an activator of microautophagy, is selected from a group comprising (2′Z,3′E)-6-Bromoindirubin-3′-oxime (6-Bio), acacetin 5,7-dihydroxy-2-(4-methoxyphenyl)chromen-4-oneN6-(4-Aminobenzyl)-9-[5-(m ethylcarbonyl)-β-D-ribofuranosyl]adenine(AB-MECA, Lapidine; [(3S,3aR,4R,8aR)-3-hydroxy-6,8a-dimethyl-8-oxo-3-propan-2-yl-2,3a,4,5-tetrahydro-1H-azulen-4-yl](E)-2-methylbut-2-enoate, Senecionine; 12-Hydroxysenecionan-11,16-dione, XCT790; (3-[4-(2,4-Bis-trifluoromethylbenzyloxy)-3-methoxyphenyl]-2-cyano-N-(5-trifluoromethyl-1,3,4-thiadiazol-2-yl)acrylamide), PD180970; (6-(2,6-Dichlorophenyl)-2-[(4-fluoro-3-methylphenyl)amino]-8-methyl-pyrido[2,3-d]pyrimidin-7(8H)-one), Ritodrine hydrochloride; (N-(p-Hydroxyphenethyl)-4-hydroxynorephedrine hydrochloride) and SB 242084 dihydrochloride hydrate; (6-Chloro-2,3-dihydro-5-methyl-N-[6-[(2-methyl-3-pyridinyl)oxy]-3-pyridinyl]-1H-indole-1-carboxyamide dihydrochloride hydrate), or any combination thereof.

In an embodiment, the modulator in the method of the present disclosure induces autophagy including but not limiting to macroautophagy, chaperone mediated autophagy and microautophagy, thereby enhances or increases autophagic flux

In another embodiment, the modulator in the method of the present disclosure while modulating autophagy restores homeostasis, particularly restores cellular homeostasis.

In an embodiment, the method enhances starvation induced autophagy including but not limiting to macroautophagy, chaperone mediated autophagy and microautophagy.

In an embodiment, the method enhances autolysosme formation in autophagy including but not limiting to macroautophagy, chaperon mediated autophagy and microautophagy.

In an embodiment, the method increases autolysosome number(s) by about 8 fold to about 12 fold in autophagy when compared to an autophagy devoid of modulator of the present disclosure, indicating enhanced fusion of autophagosomes with lysosomes by the modulator of autophagy in the method of the of the present disclosure.

In an embodiment, the method causes about 2 fold to about 4 fold increase in the autophagic flux when compared to the autophagy which is not driven by the method of the present disclosure.

In an embodiment, the method of the present disclosure enhances formation of autolysosome and increases autophagic flux during starvation or growth or both.

In a non-limiting embodiment, the method of the present disclosure modulates autophagy selected from a group comprising mitophagy (degradation of mitochondria), pexophagy (degradation of peroxisomes), lipophagy (degradation of lipid), reticulophagy (degradation of endoplasmic reticulum), ribophagy (degradation of ribosome), zymophagy (degradation of secretory granules), aggrephagy (degradation of protein aggregates), nucleophagy (degradation of nuclear parts) and xerophagy (degradation of pathogens), or any combinations thereof.

In an embodiment, the method of the present disclosure leads to about 2 fold to about 4 fold increase in aggrephagy (protein degradation) when compared to the autophagy not driven by the method of the present disclosure.

In another embodiment, the method of the present disclosure leads to about 2 fold increase in mitophagy (degradation of mitochondria), pexophagy (degradation of peroxisomes), lipophagy (degradation of lipid), reticulophagy (degradation of endoplasmic reticulum), ribophagy (degradation of ribosome), zymophagy (degradation of secretory granules), nucleophagy (degradation of nuclear parts) or xenophagy (degradation of pathogens)when compared to the autophagy not driven by the method of the present disclosure.

In an embodiment, the method of the present disclosure while modulating does not affect the normal functioning of the cell or tissue or combination thereof.

In another embodiment, the method of the present disclosure while modulating autophagy does not affect cell viability and growth of cell or tissue or combination thereof.

In an embodiment, the method of the present disclosure enhances basal autophagy and induced autophagy, independently or in combination.

In an embodiment, in the method of the present disclosure, the modulator, 6-Bio enhances starvation induced autophagy or growth.

In an exemplary embodiment, in the method of the present disclosure, (2′Z,3′E)-6-Bromoindirubin-3′-oxime (6-Bio) modulates autophagy including but not limiting to mitophagy (degradation of mitochondria), pexophagy (degradation of peroxisomes), lipophagy (degradation of lipid), reticulophagy (degradation of endoplasmic reticulum), ribophagy (degradation of ribosome), zymophagy (degradation of secretory granules), aggrephagy (degradation of protein aggregates) and xenophagy.

In another exemplary embodiment, in the method of the present disclosure acacetin modulates autophagy including but not limiting to mitophagy (degradation of mitochondria), pexophagy (degradation of peroxisomes), lipophagy (degradation of lipid), reticulophagy (degradation of endoplasmic reticulum), ribophagy (degradation of ribosome), zymophagy (degradation of secretory granules), Aggrephagy (degradation of protein aggregates) and xenophagy.

In an embodiment, in the method of the present disclosure the 6-Bio induces macroautophagy leading to increased autophagic flux resulting in aggregate degradation or clearance of protein aggregate including but not limiting to α-synuclein within a cell or tissue or both.

In an embodiment, in the method of the present disclosure, the 6-Bio rescues growth lag due to α-synuclein toxicity in yeast as opposed to other GSK-3 inhibitors known in the art.

In another embodiment, in the method of the present disclosure, the 6-Bio shows increased efficiency in activation of autophagy compared to known neuro protective compounds such as Agk2.

In a further embodiment, in the method of the present disclosure, the 6-Bio enhances starvation induced autophagy more efficiently as compared to other known neuro-protective compounds such as Agk2.

In exemplary embodiment, in the method of the present disclosure the 6-Bio activates autophagy including but not limiting to aggrephagy, wherein the 6-Bio clears or leads to degradation of α-synuclein aggregates and restores cellular homeostasis.

In exemplary embodiment, the method of the present disclosure manages neurodegenerative disorder including but not limiting to Alzheimer's disease, Amyotrophic lateral sclerosis, Friedreich's ataxia, Huntington's disease, Lewy body disease, Parkinson's disease and Spinal muscular atrophy.

In another exemplary embodiment, the method of the present disclosure treats neurodegenerative disorder including but not limiting to Alzheimer's disease, Amyotrophic lateral sclerosis, Friedreich's ataxia, Huntington's disease, Lewy body disease, Parkinson's disease and Spinal muscular atrophy.

In a non-limiting embodiment, in the method of the present disclosure the 6-Bio treats neurodegenerative disorder including but not limiting to Alzheimer's disease, Amyotrophic lateral sclerosis, Friedreich's ataxia, Huntington's disease, Lewy body disease, Parkinson's disease and Spinal muscular atrophy.

In another non-limiting embodiment, in the method of the present disclosure, the 6-Bio halts neurodegeneration, unlike commonly administered drugs such as L-DOPA for Parkinson's disease.

In another embodiment, the 6-Bio in the method of the present disclosure inhibits Glycogen synthase kinase 3 beta (GSK3B) function.

In a further embodiment, the 6-Bio in the method of the present disclosure shows increased autophagy induction than GSK3B inhibitors.

In another embodiment, in the method of the present disclosure 6-Bio modulates GSK3B, PDK1 and Jak/STAT3 signaling pathways.

In a further embodiment, in the method of the present disclosure 6-Bio prevents cytotoxicity by restoring cellular proteostasis.

In an alternate embodiment, the method of the present disclosure inhibits intracellular growth of the microorganisms including but not limiting to Salmonella typhimurium, Legionella pneumophila, Listeria monocytogenes, Shigella flexneri, Streptococcus pyrogenes and Mycobacterium tuberculosis, while modulating autophagy.

In another alternate embodiment, in the method of the present disclosure acacetin inhibits the intracellular growth of the microorganism including but not limiting to Salmonella typhimurium Legionella pneumophila, Listeria monocytogenes, Shigella flexneri, Streptococcus pyrogenes and Mycobacterium tuberculosis.

In another alternate embodiment, the acacetin enhances pexophagy.

In an embodiment, in the method of the present disclosure the concentration of 6-bio that modulates autophagy including but not limiting to macroautophagy, chaperone mediated autophagy and microautophagy, ranges from about 5 μM to about 150 μM.

In an embodiment, in the method of the present disclosure the concentration of 6-bio that modulates autophagy including but not limiting to macroautophagy, chaperone mediated autophagy and microautophagy, ranges from about 40 μM to about 150 μM, preferably about 50 μM in yeast cells.

In another embodiment, in the method of the present disclosure the concentration of Acacetin that modulates autophagy including but not limiting to macroautophagy, chaperone mediated autophagy and microautophagy, ranges from about 25 μl to about 50 μM.

In another embodiment, in the method of the present disclosure the concentration of 6-bio that modulates autophagy in mammalian cells ranges from about 5 μM to about 50 μM, preferably about 5 μM.

In a further embodiment, in the method of the present disclosure the concentration of Acacetin that modulates autophagy in yeast cells ranges from about 25 μM to about 50 μM, preferably about 50 μM.

In another embodiment, in the method of the present disclosure the concentration of Acacetin that modulates autophagy including but not limiting to macroautophagy, chaperone mediated autophagy and microautophagy in yeast cells is 25 μM, 25.5 μM, 30 μM, 30.5 μM, 31 μM, 31.5 μM, 32 μM, 32.5 μM, 33 μM, 33.5 μM, 34 μM, 34.5 μM, 35 μM, 35.5 μM, 36 μM, 36.5 μM, 37 μM, 37.5 μM, 38 μM, 38.5 μM, 39 μM, 39.5 μM, 40 μM, 40.5 μM, 41 μM, 41.5 μM, 42 μM, 42.5 μM, 43 μM, 43.5 μM, 44 μM, 44.5 μM, 45 μM, 45.5 μM, 46 μM, 46.5 μM, 47 μM, 47.5 μM, 48 μM, 48.5 μM, 49 μM, 49.5 or 50 μM.

In another embodiment, in the method of the present disclosure the concentration of Acacetin that modulates autophagy in mammalian cells ranges from about 25 μM to about 50 μM, preferably about 50 μM in mammalian cells.

In another embodiment, Acacetin can be used in the management of bacterial infections.

In an exemplary embodiment, in the method of the present disclosure, 3-[4-(2,4-Bis-trifluoromethylbenzyloxy)-3-methoxyphenyl]-2-cyano-N-(5-trifluoromethyl-1,3,4-thiadiazol-2-yl)acrylamide (XCT790) modulates autophagy including but not limiting to mitophagy (degradation of mitochondria), pexophagy (degradation of peroxisomes), lipophagy (degradation of lipid), reticulophagy (degradation of endoplasmic reticulum), ribophagy (degradation of ribosome), zymophagy (degradation of secretory granules), aggrephagy (degradation of protein aggregates) and xenophagy.

In an embodiment, in the method of the present disclosure the XCT-790 induces macroautophagy leading to increased autophagic flux resulting in aggregate degradation or clearance of protein aggregate including but not limiting to α-synuclein within a cell or tissue or both.

In an embodiment, in the method of the present disclosure, the XCT-790 rescues growth lag due to α-synuclein toxicity in yeast as opposed to untreated condition.

In another embodiment, in the method of the present disclosure, the XCT-790 shows increased efficiency in activation of autophagy compared to known neuro-protective compounds such as Agk2.

In exemplary embodiment, in the method of the present disclosure the XCT-790 activates autophagy including but not limiting to aggrephagy, wherein the XCT-790 clears or leads to degradation of a-,synuclein aggregates and restores cellular homeostasis.

In a non-limiting embodiment, in the method of the present disclosure the XCT-790 treats neurodegenerative disorder including but not limiting to Alzheimer's disease, Amyotrophic lateral sclerosis, Friedreich's ataxia, Huntington's disease, Lewy body disease, Parkinson's disease and Spinal muscular atrophy.

In an embodiment, in the method of the present disclosure the concentration of XCT-790 that modulates autophagy including but not limiting to macroautophagy, chaperone mediated autophagy and microautophagy, ranges from about 5 μM to about 150 μM.

In an embodiment, in the method of the present disclosure the concentration of XCT-790 that modulates autophagy including but not limiting to macroautophagy, chaperone mediated autophagy and microautophagy, ranges from about 40 μM to about 150 μM, preferably about 50 μM in yeast cells.

In another embodiment, in the method of the present disclosure the concentration of XCT-790 that modulates autophagy in mammalian cells ranges from about 5 μM to about 50 μM, preferably about 5 μM.

In an embodiment, in the method of the present disclosure, XCT-790 modulates autophagosome formation in an ERRα dependent manner.

In an embodiment, in the method of the present disclosure, the XCT-790 modulates fusion of autophagaosome to lysosome in an ERRα dependent manner.

In an embodiment, the ERRα is localized on to the autophagosomes and upon autophagy induction by XCT-790 by the method of the present disclosure, localization is lost and it is accompanied with an increase in autophagosome biogenesis.

In an embodiment, in the method of the present disclosure, XCT-790 clears α-synuclein (SNCA) aggregates in an autophagy-dependent manner in both yeast and human neuronal cells. The XCT-790 significantly induces autophagy through an mTOR-independent mechanism and ERRα-dependent mechanism.

In an embodiment, the present disclosure relates to a method of modulating autophagy, wherein the method comprising step of contacting cell with modulator of autophagy, wherein the modulator is an inhibitor of autophagy including but not limiting to macroautophagy, chaperon mediated autophagy and microautophagy.

In an exemplary embodiment, in the method of the present disclosure, the modulator which is an inhibitor of autophagy is selected from a group comprising N-Carbobenzyloxy-L-phenylalanylchloromethyl ketone (ZPCK), 3-[(4-methylphenyl)sulfonyl]-(2E)-propenenitrile (Bay11-7082), Elaidylphosphocholine, N-[4-(1H-Benzimidazol-2-yl)phenyl]-5-nitro-2-thiophenecarboxamide, Ethyl [(2-{[(5-nitro-2-thienyl)carbonyl]amino}-3-thienyl)carbonyl]carbamate, 2-Phenyl-N-[5-(3-thienyl)-1,3,4-oxadiazol-2-yl]-2H-1,2,3-triazole-4-carboxamide, 3-Bromo-N-[5-(5,6-dihydro-1,4-dioxin-2-yl)-1,3,4-oxadiazobenzamide, N-(6-Chloro-1,3-benzothiazol-2-yl)-2-(4-fluorophenyl)-N-(3-pyridinylmethyl)acetamide, N-(2-Methoxybenzyl)-5-nitro-4,6-pyrimidinediamine, N-[5-(2,5-Dichloro-3-thienyl-1,3,4-oxadiazol-2-yl]-5-nitro-2-furamide, N-(2,5-Dimethylphenyl)-2-{[3-(3-methoxyphenyl)-4-oxo-3,4,6,7-tetrahydrothieno[3,2-d]pyrimidin-2-yl]sulfanyl}acetamide, 4-Fluoro-N-[2-(4-fluorophenyl)-2-(4-methyl-1-piperazinyl)ethyl]-3-methylbenzenesulfonamide, N-[5-(3,4-Dimethylphenyl)-1,3,4-oxadiazol-2-yl]-5-nitro-2-furamide, N[3-(1,3-Benzothiazol-2-yl)-6-methyl-4,5,6,7-tetrahydrothieno[2,3-c]pyridin-2-yl]-4-cyanobenzamide hydrochloride (1:1), N-(4,5-Diphenyl-1,3-thiazol-2-yl)-1,2-oxazole-5-carboxamide, 2-(5-(2,4-Difluorophenyl)-1,2-oxazol-3-yl]-N-(1,2-oxazol-3-yl)acetamide, 1-(2,4-Dihydroxy-3-methylphenyl)-2-(4-propylphenoxy)ethanone, N-[5-(2,4-Dimethylphenyl)-1,3,4-oxadiazol-2-yl]-3,5-dimethoxybenzamide. N-[3-(1,3-Benzothiazol-2-yl)-6-ethyl-4,5,6,7-tetrahydrothieno[2,3-c]pyridin-2-yl]-1-methyl-1H-pyrazole-3-carboxamide hydrochloride (1:1), 2-(2-Bromobenzyl)-1,2,3,4-tetrahydro-3-isoquinolinecarboxamide, 2-[(5,7-Dibromo-8-quinolinyl)oxy]propanamide, 3-Amino-N-[4-(imidazo[1,2-a]pyridin-2-yl)phenyl]-2-pyrazinecarboxamide, 1-Benzyl-N-[5-chloro-2-(1-piperidinyl)phenyl]-1H-1,2,3-triazole-4-carboxamide, (4-Bromophenyl){4-[(5-chloro-2-thienyl)sulfonyl]-1-piperazinyl}methanone, 1-Phenyl-N-(1,3,4-thiadiazol-2-yl)-3-(2-thienyl)-1H-pyrazole-5-carboxamide, 5-{-[(7-Bromo-2,3-dihydro-1,4-benzodioxin-6-yl)methyl]sulfanyl}-N-cyclopropyl-1,3,4-thiadiazol-2-amine, 1-Ethyl-7-methyl-4-oxo-1,4-dihydro-1,8-naphthyridine-3-carboxylic acid, Pentachlorophenol, N,N,N-Trimethyl-1-hexadecanaminium bromide, 1,4-Dihydroxy-5,8-bis({2-[(2-hydroxyethyl)amino]ethyl}amino)-9,10-anthraquinone dihydrochloride, (9E)-9-Octadecen-1-yl 2-(trimethylammonio)ethyl phosphate, 4-(1H-Indazol-3-yl)-N-(4-piperidinyl)-1H-pyrrolo[2,3-b]pyridin-6-amine, 2-(4-Methoxybenzyl)-6-4,5,6,7-tetrahydropyrazolo[1,5-a]pyrazin-2-ylmethyl)-6,7-dihydro-5H-pyrrolo[3,4-d]pyrimidine, [(E)-octadec-9-enyl] 2-(trimethylazaniumyl)ethyl phosphate), TNP (N6-[(4-nitrophenyl)methyl]-N2-[[3-(trifluoromethyl)phenyl]methyl]-9H-Purine-2,6-diamine), Cinchonidine; (R)-[(2S,4S,5R)-5-ethenyl-1-azabicyclo[2.2.2]octan-2-yl]-quinolin-4-ylmethanol, Trioxsalen and (2,5,9-Trimethylfuro[3,2-g]benzopyran-7-one), or any combinations thereof.

In an embodiment, in the method of the present disclosure wherein the modulator is inhibitor is ZPCK or Bay 11-7082 (Bay-11) or both.

In an embodiment, in the method of the present disclosure the modulator inhibits at least one of autophagosome biogenesis, autophagosome maturation, autophagosome-lysosome fusion, degradation of autophagic cargo inside vacuole after autophagosome lysosome fusion, or any combinations thereof, thereby decreasing autophagic flux.

In an exemplary embodiment, in the method of the present disclosure, wherein the modulator is an inhibitor of autophagy, inhibits autophagy at a step prior to the fusion of autophagosomes to the vacuole or inhibits autophagy at the step of degradation of autophagic bodies inside the vacuole, or a combination thereof.

In a non-limiting embodiment, in the method of the present disclosure the N-Benzyloxycarbonyl-Lphenylalaninylchloromethyl ketone (ZPCK) inhibits the degradation of autophagic bodies inside the vacuole in autophagy including but not limited to macroautophagy, chaperone mediated autophagy and microautophagy.

In another non-limiting embodiment, in the method of the present disclosure, the 3-[4-methylphenyl)sulfonyl]-(2E)-propenenitrile (Bay11-7082) inhibits at a step prior to fusion of autophagosomes to the vacuole in autophagy including but not limiting to macroautophagy, chaperone mediated autophagy and microautophagy.

In an exemplary embodiment, in the method of the present disclosure, the 3-[(4-methylphenyl)sulfonyl]-(2E)-propenenitrile (Bay11-7082) acts during the autophagosome biogenesis step in autophagy including but not limiting to macroautophagy, chaperone mediated autophagy and microautophagy.

In an exemplary embodiment, in the method of the present disclosure, the inhibitor of autophagy including but not limiting to 3-[(4-methylphenyl)sulfonyl]-(2E)-propenenitrile (Bay11-7082) inhibits autophagy including but not limiting to macroautophagy, chaperone mediated autophagy and microautophagy in a dose dependent manner.

In a non-limiting embodiment, the method of the present disclosure, wherein the modulator inhibits autophagy, manages cancer or treats cancer, or both.

In another embodiment, the method of the present disclosure, wherein the modulator inhibits autophagy, acts on the tumour cells that are highly dependent on autophagy for survival, ultimately leading to cell death.

In another embodiment, the method of the present disclosure, wherein the modulator inhibits autophagy acts on pathogens that use autophagy machinery for their survival,

In an embodiment, in the method of the present disclosure the concentration of ZPCK that modulates autophagy including but not limiting to macroautophagy, chaperone mediated autophagy and microautophagy ranges from about 25 μM to about 50 μM.

In an embodiment, in the method of the present disclosure the concentration of ZPCK that modulates autophagy in yeast cells ranges from about 25 μM to 50 μM, preferably about 50 μM in yeast cells.

In another embodiment, in the method of the present disclosure the concentration of ZPCK that modulates autophagy in yeast cells is 25 μM, 25.5 μM, 30 μM, 30.5 μM, 31 μM, 31.5 μM, 32 μM, 32.5 μM, 33 μM, 33.5 μM, 34 μM, 34.5 μM, 35 μM, 35.5 μM, 36 μM, 36.5 μM, 37 μM, 37.5 μM, 38 μM, 38.5 μM, 39 μM, 39.5 μM, 40 μM, 40.5 μM, 41 μM, 41.5 μM, 42 μM, 42.5 μM, 43 μM, 43.5 μM, 44 μM, 44.5 μM, 45 μM, 45.5 μM, 46 μM, 46.5 μM, 47 μM, 47.5 μM, 48 μM, 48.5 μM, 49 μM, 49.5 μM or 50 μM.

In an embodiment, in the method of the present disclosure the concentration of ZPCK that modulates autophagy in mammalian cells ranges from about 25 μM to 50 μM, preferably about 25 μM.

In another embodiment, in the method of the present disclosure the concentration of ZPCK that modulates autophagy in mammalian cells is 25 μM, 25.5 μM, 30 μM, 30.5 μM, 31 μM, 31.5 μM, 32 μM, 32.5 μM, 33 μM, 33.5 μM, 34 μM, 34.5 μM, 35 μM, 35.5 μM, 36 μM, 36.5 μM, 37 μM, 37.5 μM, 38 μM, 38.5 μM, 39 μM, 39.5 μM, 40 μM, 40.5 μM, 41 μM, 41.5 μM, 42 μM, 42.5 μM, 43 μM, 43.5 μM, 44 μM, 44.5 μM, 45 μM, 45.5 μM, 46 μM, 46.5 μM, 47 μM, 47.5 μM, 48 μM, 48.5 μM, 49 μM, 49.5 μM or 50 μM.

In an embodiment, in the method of the present disclosure the concentration of Bay-11 that modulates autophagy including but not limiting to macroautophagy, chaperone mediated autophagy and microautophagy ranges from about 1 μM to 25 μM.

In an embodiment, in the method of the present disclosure the concentration of Bay-11 that modulates autophagy in yeast cells ranges from about 1 μM to 25 μM preferably about 25 μM.

In another embodiment, in the method of the present disclosure the concentration of Bay-11 that modulates autophagy in yeast cells is 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 or 25 μM.

In an embodiment, in the method of the present disclosure the concentration of Bay-11 that modulates autophagy in mammalian cells ranges from about 1 M to 10 μM, preferably about 2.5 μM in mammalian cells.

In another embodiment, in the method of the present disclosure the concentration of Bay-11 that modulates autophagy in mammalian cells is 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM or 10 μM.

In another embodiment, in the method of the present disclosure, the ZPCK manages or treats a condition including but not limiting to cancer/tumour, condition caused by tumour cells and pathogens, in a subject in need thereof.

In another embodiment, in the method of the present disclosure, the ZPCK manages or treats a condition including but not limiting to cancer/tumour, condition caused by tumour cells and pathogens in a subject in need thereof.

In another embodiment, in the method of the present disclosure, the Bay11-7082 manages or treats a condition including but not limiting to cancer/tumour, condition caused by tumour cells and pathogen in a subject in need thereof.

In an exemplary embodiment, microscopic studies in S. cerevisiae for the degradation of peroxisomes through autophagy shows a decrease in the degradation of peroxisomes an presence of both the inhibitors of autophagy such as ZPCK and Bay11-7082 employed in the method of the present disclosure as observed through accumulation of GFP positive punctate structures (peroxisomes) inside or outside of the vacuole (labelled with FM4-64).

In another embodiment, in cells treated with autophagy inhibitor Bay11-7082 by the method of the present disclosure, peroxisomes get accumulated outside the vacuole even in starvation in the cell.

In another embodiment, ZPCK treated cells by the method of the present disclosure show build-up of peroxisomes inside the vacuole.

In a further embodiment, to elucidate the step of action of Bay 11-7082 in the method of the present disclosure, a protease protection assay is performed using aminopeptidase as a marker, which is also a substrate for autophagy on starvation; wherein the principle of the assay is that a cargo protected by a membrane is resistant to the action of proteases; with the help of a detergent like Triton X-100, the membrane is dissolved, and the cargo is made available for degradation by proteinase K treatment; untreated cells show both precursor as well as the matured form, due to both the membrane protected cargo sequestered within the autophagosome and the free form present in the cytosol respectively, when treated with only proteinase K (FIG. 17d) while Bay11-7082 treated cells primarily show only the mature form of aminopeptidase on proteinase K treatment (FIG. 17d);conversion of precursor to matured form of aminopeptidase on treatment with proteinase K in Bay 11-7082 treated cells indicates that the cargo is not protected by the autophagosome membrane and thus autophagosome biogenesis is inhibited on treatment with Bay 11-7082.

In another embodiment, traffic light assay used for studying autophagic flux is employed to assess the effect of both autophagy inhibitors Bay11-7082 and ZPCK employed in the method of the present disclosure in HeLa cells (FIG. 18a) to validate the results obtained in yeast S. cerevisiae; wherein a tandem fluorescent tagged LC3 construct is used as a reporter which has LC3 tagged to mRFP and GFP (ptfLC3); the idea behind this methodology being that due to the double tagging, autophagosomes appear yellow but when they fuse with lysosomes, the GFP fluorescence gets quenched due to low pH of lysosome, making autolysosomes appear red, giving a clear picture of the autophagic flux status of a cell; wherein induction of autophagy either due to starvation or a chemical inducer of autophagy causes significant increase in number of yellow and red dots (autophagosomes and autolysosomes, respectively); it is observed that treatment with Bay11-7082 (2.5 μM) for 2 hours decreases the number of autophagosomes (yellow dots) and autolysosomes (red dots) in ptfLC3 expressing HeLa cells (FIG. 18b) and treatment with ZPCK (25 μM), increases the number of red dots with no significant change in the yellow dots inside the cell (FIG. 4b),thus indicating that the compounds work in a similar fashion in HeLa cells as they did in the yeast counterpart,

The present disclosure further relates to modulators of autophagy for, enhancing formation of autolysosome by promoting fusion of autophagosome and lysosome, thereby increasing autophagic flux or for inhibiting at least one of autophagosome biogenesis, autophagosome maturation, autophagosome-lysosome fusion, degradation of autophagic cargo inside vacuole after autophagosome-lysosome fusion, or any combination thereof, thereby decreasing autophagic flux.

In an embodiment, the modulator is (2′Z,3′E)-6-Bromoindirubin-3′-oxime (6-Bio), acacetin, 7-dihydroxy-2-(4-methoxyphenyl)chromen-4-oneN6-(4-Aminobenzyl)-9-[5-(methylcarbonyl)-β-D-ribofuranosyl]adenine(AB-MECA, Lapidine; (3S,3aR,4R,8aR)-3-hydroxy-6,8a-dimethyl-8-oxo-3-propan-2-yl-2,3a,4,5-tetrahydro-1H-azulen-4-yl](E)-2-methylbut-2-enoate, Senecionine, 12-Hydroxysenecionan-11,16-dione, XCT790; (3-[4-(2,4-Bis-trifluoromethylbenzyloxy)-3-methoxyphenyl]-2-cyano-N-(5-trifluoromethyl-1,3,4-thiadiazol-2-yl)acrylamide), PD180970 (6-(2,6-Dichlorophenyl)-2-[(4-fluoro-3-methylphenyl)amino]-8-methyl-pyrido[2,3-d]pyrimidin-7(8H)-one), Ritodrine hydrochloride; (N-(p-Hydroxyphenethyl)-4-hydroxynorephedrine hydrochloride) and SB 242084 dihydrochloride hydrate; (6-Chloro-2,3-dihydro-5-methyl-N-[6-[(2-methyl-3-pyridinyl)oxy]-3-pyridinyl]-1H-indole-1-carboxyamide dihydrochloride hydrate), N-Carbobenzyloxy-L-phenylalanylchloromethyl ketone (ZPCK), 3-[(4-methylphenyl)sulfonyl]-(2E)-propenenitrile (Bay11-7082), Elaidylphosphocholine, N-[4-(1H-Benzimidazol-2-yl)phenyl]-5-nitro-2-thiophenecarboxamide, Ethyl [(2-{[(5-nitro-2-thienyl)carbonyl]amino}-3-thienyl)carbonyl]carbamate, 2-Phenyl-N-[5-(3-thienyl)-1,3,4-oxadiazol-2-yl]-2H-1,2,3-triazole-4-carboxamide, 3-Bromo-N-[5-(5,6-dihydro-1,4-dioxin-2-yl)-1,3,4-oxadiazol-2-yl]benzamide, N-(6-Chloro-1,3-benzothiazol-2-yl)-2-(4-fluorophenyl)-N-(3-pyridinylmethyl)acetamide, N-(2-Methoxybenzyl)-5-nitro-4,6-pyrimidinediamine, N-[5-(2,5-Dichloro-3-thienyl)-1,3,4-oxadiazol-2-yl]-5-nitro-2-furamide, N-(2,5-Dimethylphenyl)-2-{[3-(3-methoxyphenyl)-4-oxo-3,4,6,7-tetrahydrothieno[3,2-d]pyrimidin-2-yl]sulfanyl}acetamide, 4-Fluoro-N-[2-(4-fluorophenyl)-2-(4-methyl-1-piperazinyl)ethyl]-3-methylbenzenesulfonamide, N-[5-3,4-Dimethylphenyl)-1,3,4-oxadiazol-2-yl]-5-nitro-2-furamide, N-[3-(1,3-Benzothiazol-2-yl)-6-methyl-4,5,6,7-tetrahydrothieno[2,3-c]pyridin-2-yl]-4-cyanobenzamide hydrochloride (1:1), N-(4,5-Diphenyl-1,3-thiazol-2-yl)-1,2-oxazole-5-carboxamide, 2-[5-(2,4-Difluorophenyl)-1,2-oxazol-3-yl]-N-(1,2-oxazol-3-yl)acetamide, 1-(2,4-Dihydroxy-3-methylphenyl)-2-(4-propylphenoxy)ethanone, N-[5-(2,4-Dimethylphenyl)-1,3,4-oxadiazol-2-yl]-3,5-dimethoxybenzamide, N-[3-(1,3-Benzothiazol-2-yl)-6-ethyl-4,5,6,7-tetrahydrothieno[2,3-c]pyridin-2-yl]-1-methyl-1H-pyrazole-3-carboxamide hydrochloride (1:1), 2-(2Bromobenzyl)-1,2,3,4-tetrahydro-3-isoquinolinecarboxamide, 2-[(5,7-Dibromo-8-quinolinyl)oxy]propanamide, 3-Amino-N -[4-(imidazo[1,2-a]pyridin-2-yl)phenyl]-2-pyrazinecarboxamide, 1-Benzyl-N-[5-chloro-2-(1-piperidinyl)phenyl]-1H-1,2,3-triazole-4-carboxamide, (4-Bromophenyl){4-[(5-chloro-2-thienyl)sulfonyl]-1-piperazinyl}methanone, 1-Phenyl-N-(1,3,4-thiadiazol-2-yl)-3-(2-thienyl)-1H-pyrazole-5-carboxamide, 5-{[(7-Bromo-2,3-dihydro-1,4-benzodioxin-6-yl)methyl]sulfanyl}-N-cyclopropyl-1,3,4-thiadiazol-2-amine, 1-Ethyl-7-methyl-4-oxo-1,4-dihydro-1,8-naphthyridine-3-carboxylic acid, Pentachlorophenol, N,N,N-Trimethyl-1-hexadecanaminium bromide, 1,4-Dihydroxy-5,8-bis({2-[(2-hydroxyethyl)amino]ethyl}amino)-9,10-anthraquinone dihydrochloride, (9E)-9-Octadecen-1-yl 2-(trimethylammonio)ethyl phosphate, 4-(1H-Indazol-3-yl)-N-(4-piperidinyl)-1H-pyrrolo[2,3-b]pyridin-6-amine, 2-(4-Methoxybenzyl)-6-(4,5,6,7-tetrahydropyrazolo[1,5-a]pyrazin-2-ylmethyl)-6,7-dihydro-5H-pyrrolo[3,4-d]pyrimidine, [(E)-octadec-9-enyl] 2-(trimethylazaniumyl)ethyl phosphate), TNP (N6-[(4-nitrophenyl)methyl]-N2-[[3-trifluoromethyl)phenyl]methyl]-9H-Purine-2,6-diamine), Cinchonidine; (R)-[(2S,4S,5R)-5-ethenyl-1-azabicyclo[2.2.2]octan-2-yl]-quinolin-4-ylmethanol, Trioxsalen; (2,5,9-Trimethylfuro[3,2-g]benzopyran-7-one) or any combination thereof.

In an embodiment, the modulator of autophagy for enhancing autolysosmes formation in the cell is 6-Bio or XCT-790 or both, wherein the 6-Bio or the XCT-790 or both causes degradation of protein selected from a group comprising α-synuclein (SNCA). During modulation of autophagy, the 6-Bio is mTOR dependent and GSK3B dependent, the XCT-790 is mTOR independent and ERRα dependent and the XCT-790 is an inverse agonist of ERRα.

In an embodiment, the modulator of autophagy increases autolysosomes numbers by about 8 fold to 12 fold. In another embodiment, the modulator of autophagy causes about 2 fold to 4 fold increase in the autophagic flux.

The present disclosure further relates to modulators of autophagy wherein the modulator enhances formation of autolysosome by promoting fusion of autophagosome and lysosome, thereby increasing autophagic flux or inhibits at least one of autophagosome biogenesis, autophagosome maturation, autophagosome-lysosome fusion, degradation of autophagic cargo inside vacuole after autophagosome-lysosome fusion, or any combination thereof, thereby decreasing autophagic flux.

In an embodiment, the modulator is selected from a group comprising (2′Z,3′E)-6-Bromoindirubin-3′-oxime (6-Bio), acacetin, 7-dihydroxy-2-(4-methoxyphenyl)chromen-4-oneN6-(4-Aminobenzyl)-9-[5-(methylcarbonyl)-β-D-ribofuranosyl]adenine(AB-MECA, Lapidine; (3S,3aR,4R, 8aR)-3-hydroxy-6,8a-dimethyl-8-oxo-3-propan-2-yl-2,3a,4,5-tetrahydro-1H-azulen-4-yl](E)-2-methylbut-2-enoate, Senecionine, 12-Hydroxysenecionan-11,16-dione, XCT790; (3-[4-(2,4-Bis-trifluoromethylbenzyloxy)-3-methoxyphenyl]-2-cyano-N-(5-trifluoromethyl-1,3,4-thiadiazol-2-yl)acrylamide), PD180970 (6-(2,6-Dichlorophenyl)-2-[(4-fluoro-3-methylphenyl)amino]-8-methyl-pyrido[2,3-d]pyrimidin-7(8H)-one), Ritodrine hydrochloride; (N-(p-Hydroxyphenethyl)-4-hydroxynorephedrine hydrochloride) and SB 242084 dihydrochloride hydrate; (6-Chloro-2,3-dihydro-5-methyl-N-[6-[(2-methyl-3-pyridinyl)oxy]-3-pyridinyl]-1H-indole-1-carboxyamide dihydrochloride hydrate), N-Carbobenzyloxy-L-phenylalanylchloromethyl ketone (ZPCK), 3-[(4-methylphenyl)sulfonyl]-(2E)-propenenitrile (Bay11-7082), Elaidylphosphocholine, N-[4-(1H-Benzimidazol-2-yl)phenyl]-5-nitro-2-thiophenecarboxamide, Ethyl [(2-{[(5-nitro-2-thienyl)carbonyl]amino}-3-thienyl)carbonyl]carbamate, 2-Phenyl-N-[5-(3-thienyl)-1,3,4-oxadiazol-2-yl]-2H-1,2,3-triazole-4-carboxamide, 3-Bromo-N-[5-(5,6-dihydro-1,4-dioxin-2-yl)-1,3,4-oxadiazol-2-yl]benzamide, N-(6-Chloro-1,3-benzothiazol-2-yl)-2-(4-fluorophenyl)-N-(3-pyridinylmethyl)acetamide, N-(2-Methoxybenzyl)-5-nitro-4,6-pyrimidinediamine, N-[5-(2,5-Dichloro-3-thienyl)-1,3,4-oxadiazol-2-yl]-5-nitro-2-furamide, N-(2,5-Dimethylphenyl)-2-{[3-(3-methoxyphenyl)-4-oxo-3,4,6,7-tetrahydrothieno[3,2-d]pyrimidin-2-yl]sulfanyl}acetamide, 4-Fluoro-N-[2-(4-fluorophenyl)-2-(4-methyl-1-piperazinyl)ethyl]-3-methylbenzenesulfonamide, N-[5-(3,4-Dimethylphenyl)-1,3,4-oxadiazol-2-yl]-5-nitro-2-furamide, N-[3-(1,3-Benzothiazol-2-yl)-6-methyl-4,5,6,7-tetrahydrothieno[2,3-c]pyridin-2-yl]-4-cyanobenzamide hydrochloride (1:1), N-(4,5-Diphenyl-1,3-thiazol-2-yl)-1,2-oxazole-5-carboxamide, 2-[5-(2,4-Difluorophenyl)-1,2-oxazol-3-yl]-N-(1,2-oxazol-3-yl)acetamide, 1-(2,4-)Dihydroxy-3-methylphenyl)-2-(4-propylphenoxy)ethanone, N-[5-(2,4-Dimethylphenyl)-1,3,4-oxadiazol-2-yl]-3,5-dimethoxybenzamide, N-[3-(1,3-Benzothiazol-2-yl)-6-ethyl-4,5,6,7-tetrahydrothieno[2,3-c]pyridin-2-yl]-1-methyl-1H-pyrazole-3-carboxamide hydrochloride (1:1), 2-(2-Bromobenzyl-1,2,3,4-tetrahydro-3-isoquinolinecarboxamide, 2-[(5,7-Dibromo-8-quinolinyl)oxy]propanamide, 3-Amino-N-[4-(imidazo[1,2-a]pyridin-2-yl)phenyl]-2-pyrazinecarboxamide, 1-Benzyl-N-[5-chloro-2-(1-piperidinyl)phenyl]-1H-1,2,3-triazole-4-carboxamide, (4-Bromophenyl){4-[(5-chloro-2-thienyl)sulfonyl]-1-piperazinyl}methanone, 1-Phenyl-N-(1,3,4-thiadiazol-2-yl)-3-(2-thienyl)-1H-pyrazole-5-carboxamide, 5-{[(7-Bromo-2,3-dihydro-1,4-benzodioxin-6-yl)methyl]sulfanyl}-N-cyclopropyl-1,3,4-thiadiazol-2-amine, 1-Ethyl-7-methyl-4-oxo-1,4-dihydro-1,8-naphthyridine-3-carboxylic acid, Pentachlorophenol, N,N,N-Trimethyl-1-hexadecanaminium bromide, 1,4-Dihydroxy-5,8-bis({2-[(2-hydroxyethyl)amino]ethyl}amino)-9,10-anthraquinone dihydrochloride, (9E)-9-Octadecen-1-yl 2-(trimethylammonio)ethyl phosphate, 4-(1H-Indazol-3-yl)-N-(4-piperidinyl)-1H-pyrrolo[2,3-b]pyridin-6-amine, 2-(4-Methoxybenzyl)-6-(4,5,6,7-tetrahydropyrazolo[1,5-a]pyrazin-2-ylmethyl)-6,7-dihyrdro-5H-pyrrolo[3,4-d]pyrimidine, [(E)-octadec-9-enyl] 2-(trimethylazaniumyl)ethyl phosphate), TNT (N6-[(4-nitrophenyl)methyl]-N2-[[3-(trifluoromethyl)phenyl]methyl]-9H-Purine-2,6-diamine), Cinchonidine; (R)-[(2S,4S,5R)-5-ethenyl-1-azabicyclo[2.2.2]octan-2-yl]-quinolin-4-ylmethanol, Trioxsalen; (2,5,9-Trimethylfuro[3,2-g]benzopyran-7-one) or any combination thereof.

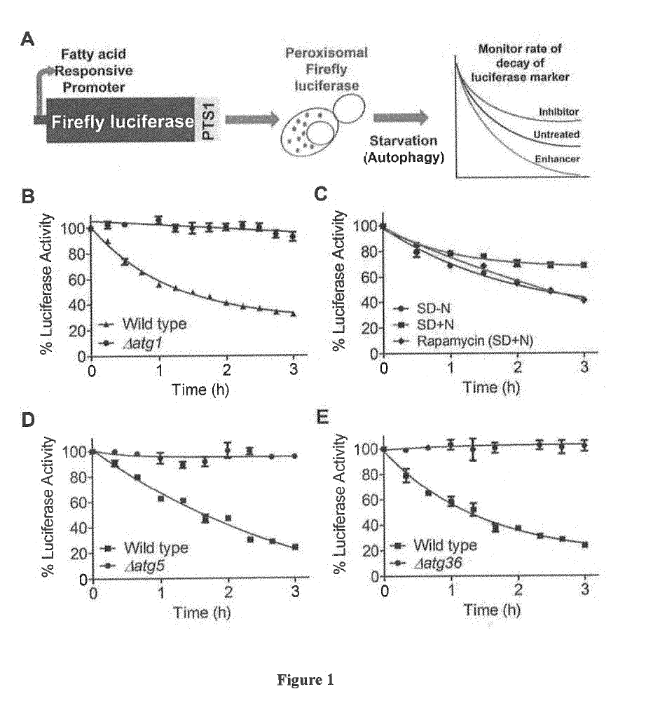

In an embodiment of the present disclosure, FIG. 1 shows the dual luciferase assay for screening of small molecules employing firefly and Renilla luciferase genes to monitor autophagy in real time, wherein wild type cells show a gradual decrease in luciferase counts upon induction of autophagy whereas core autophagy mutants atg1 and atg5 and selective autophagy mutant atg36 (adaptor protein for pexophagy) do not show any drop in the luciferase activity over time,

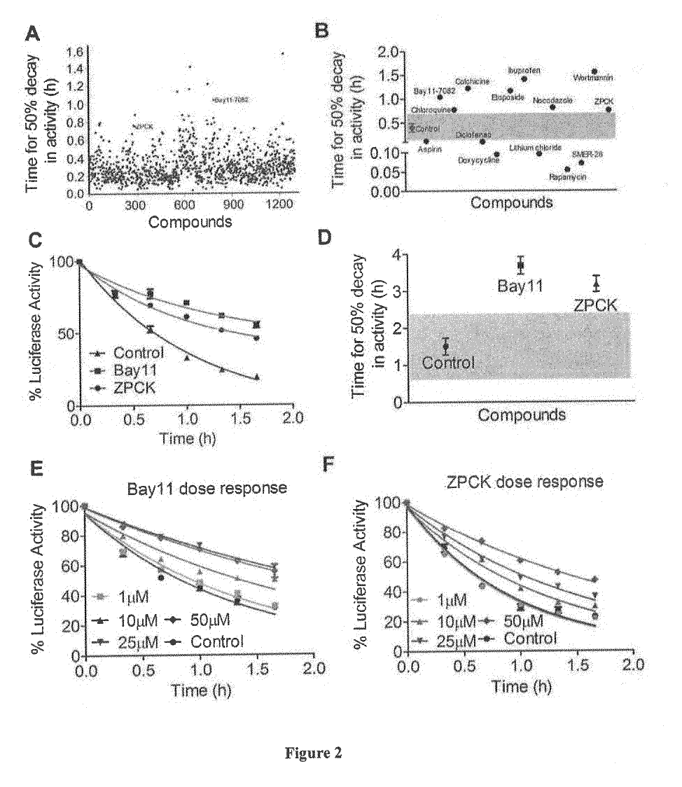

In another embodiment of the present disclosure, FIG. 2 shows screening of two small molecule libraries luciferase based assay for monitoring autophagy; wherein two putative inhibitors, Bay11-7082 and ZPCK are obtained from the primary screening and are further confirmed using luciferase assay done in triplicates; wherein a dose dependent effect of said inhibitor on the rates of degradation of firefly luciferase seen in both the compounds.

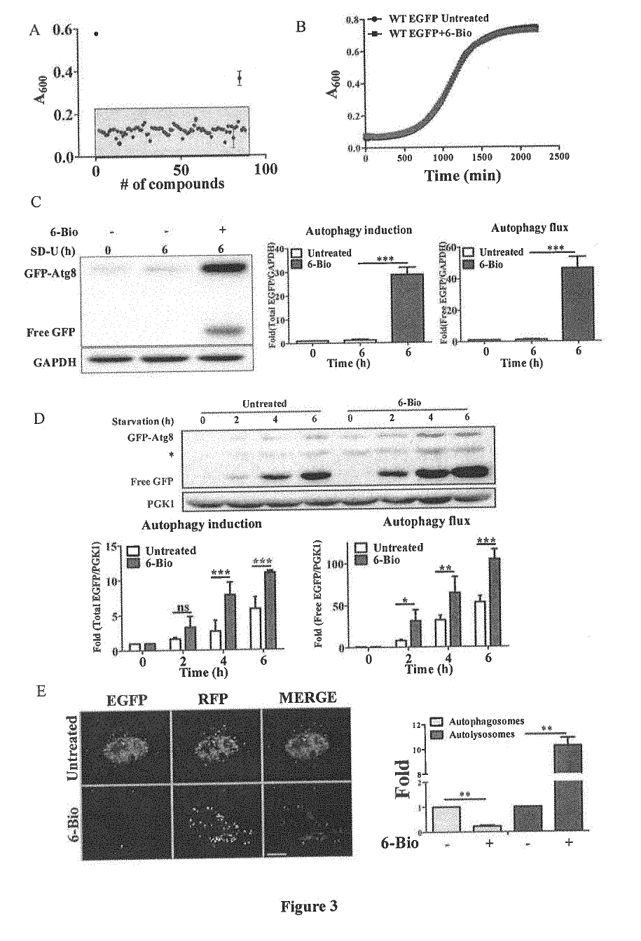

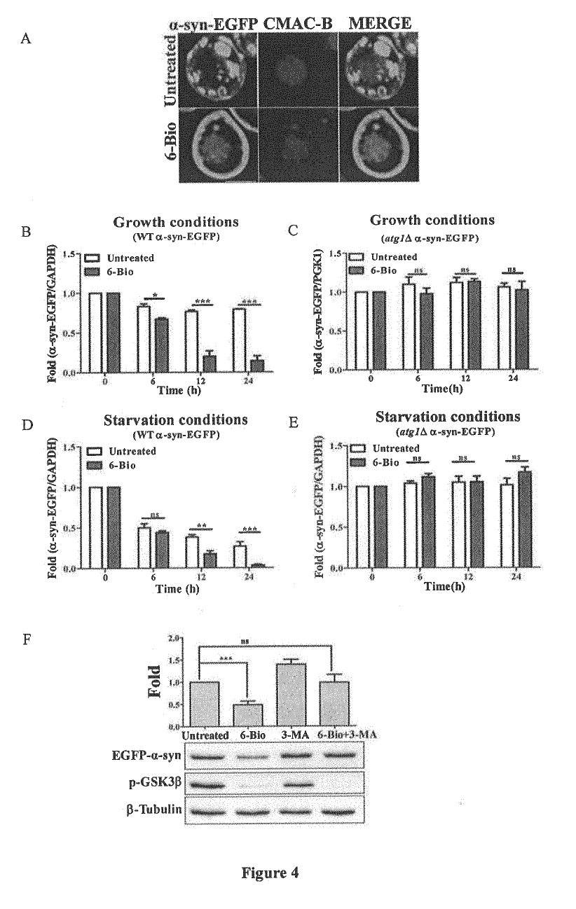

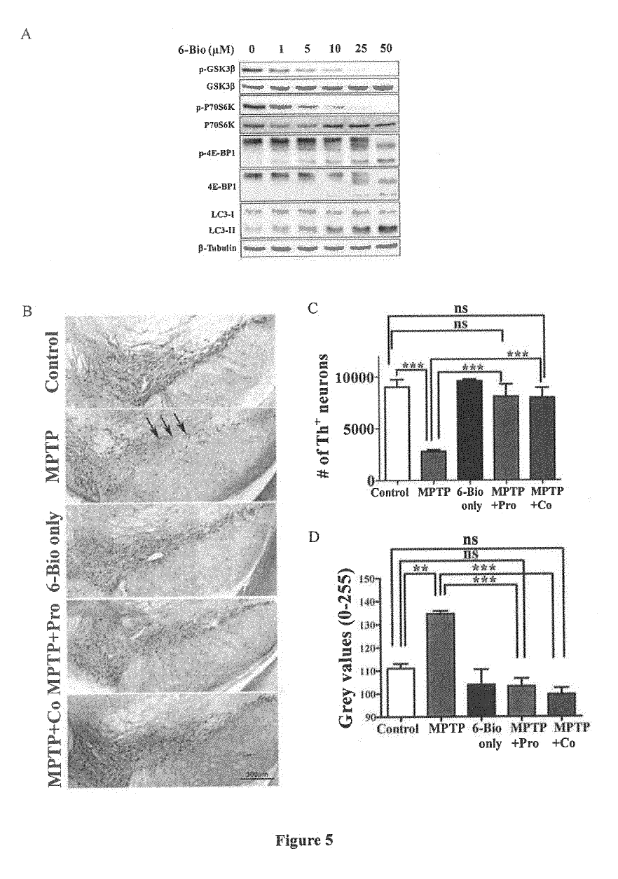

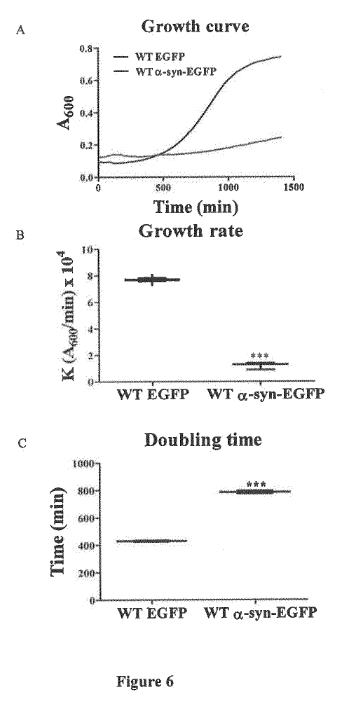

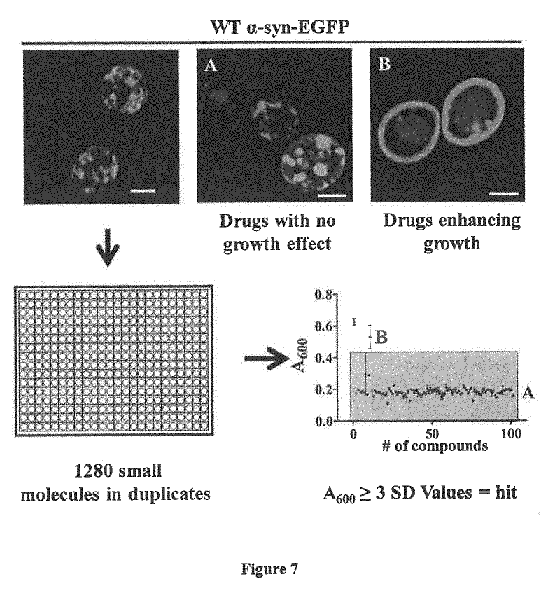

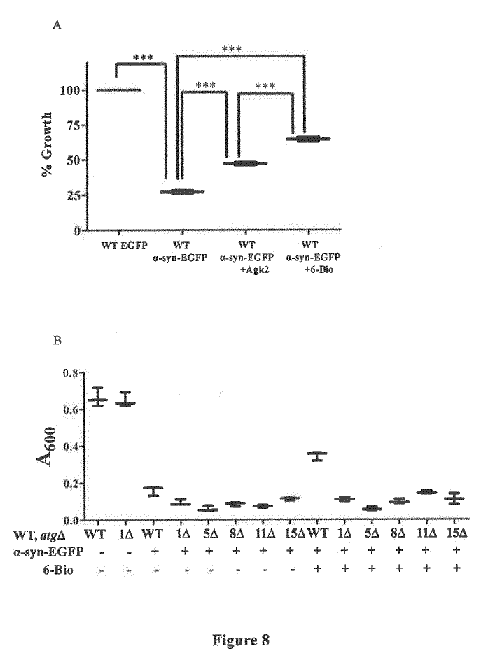

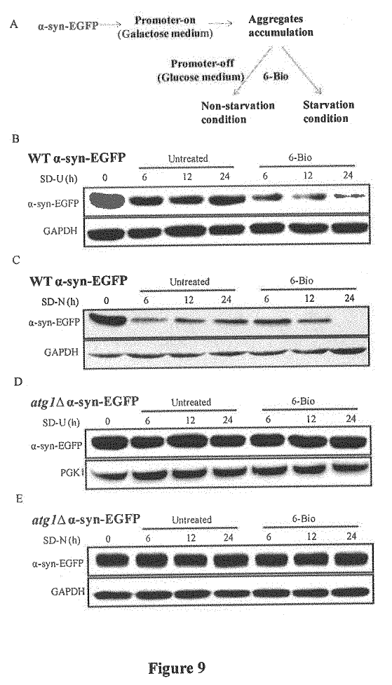

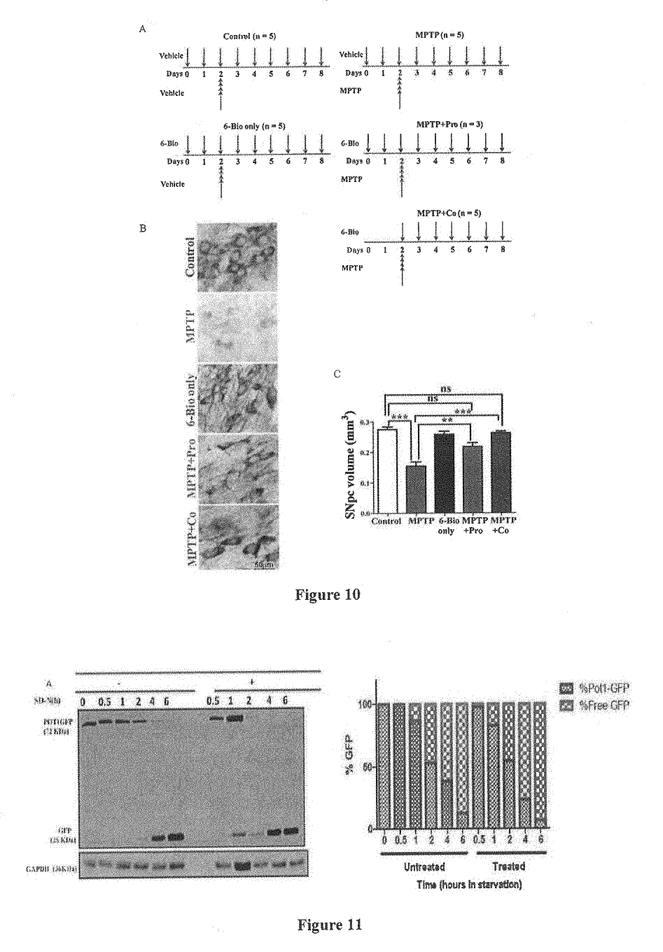

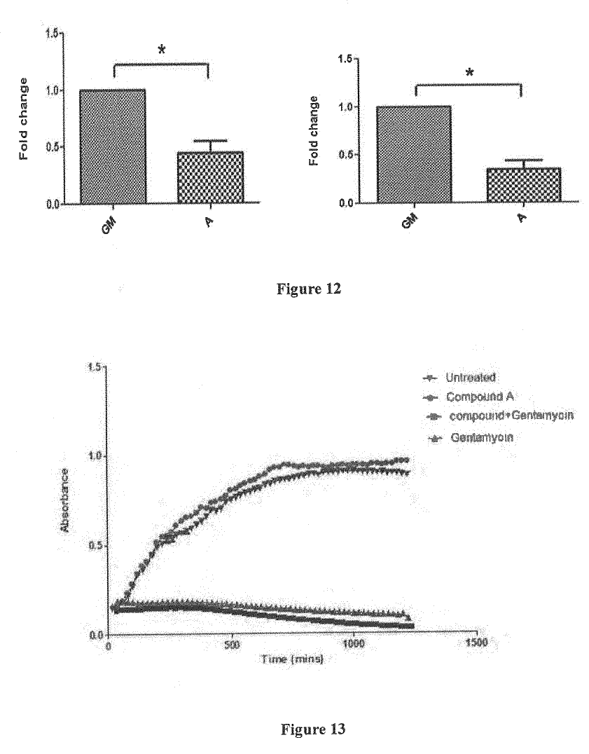

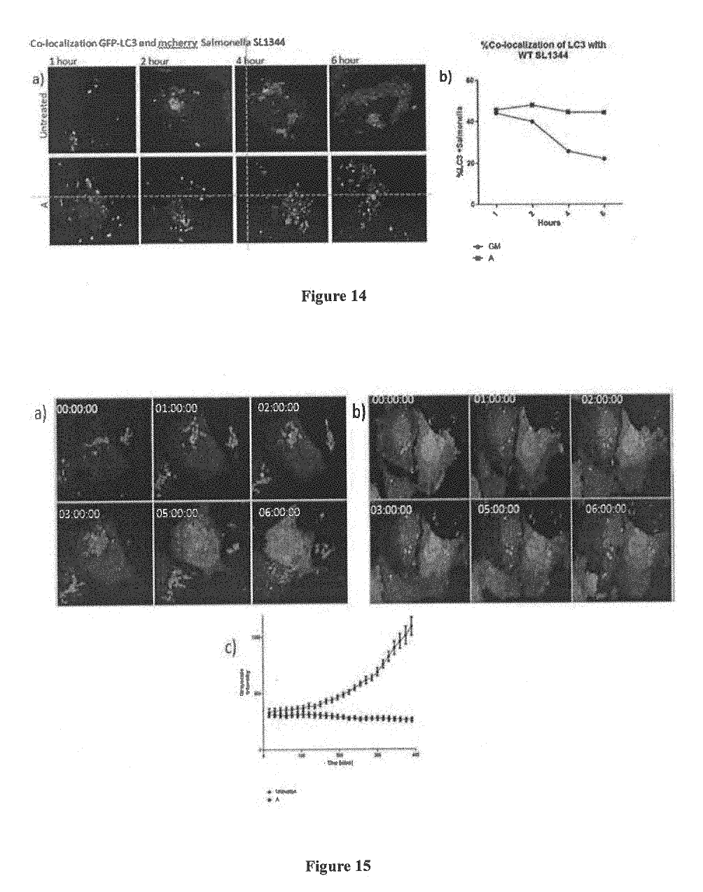

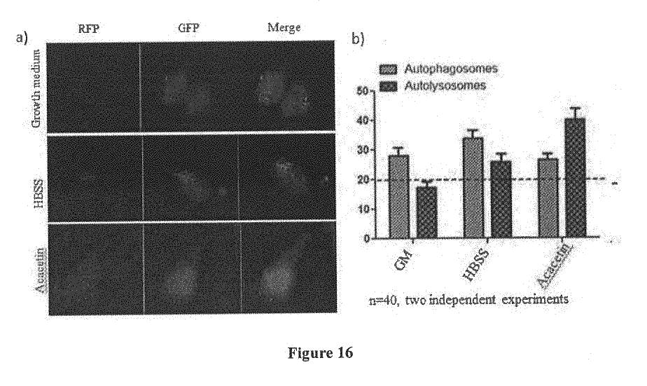

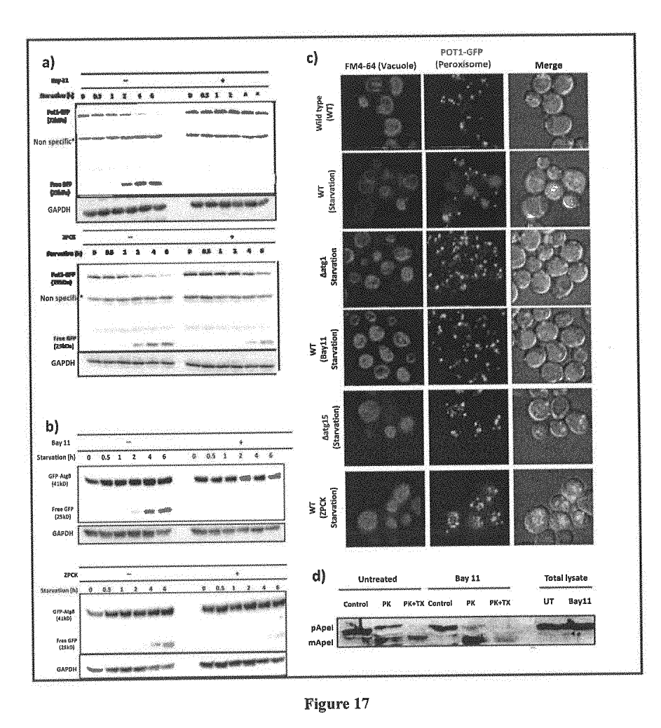

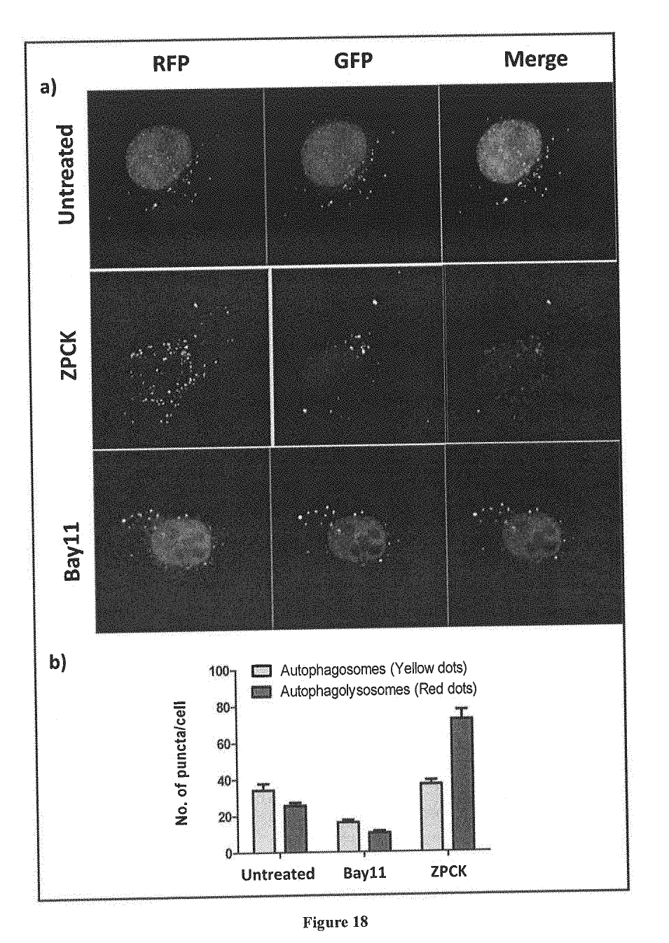

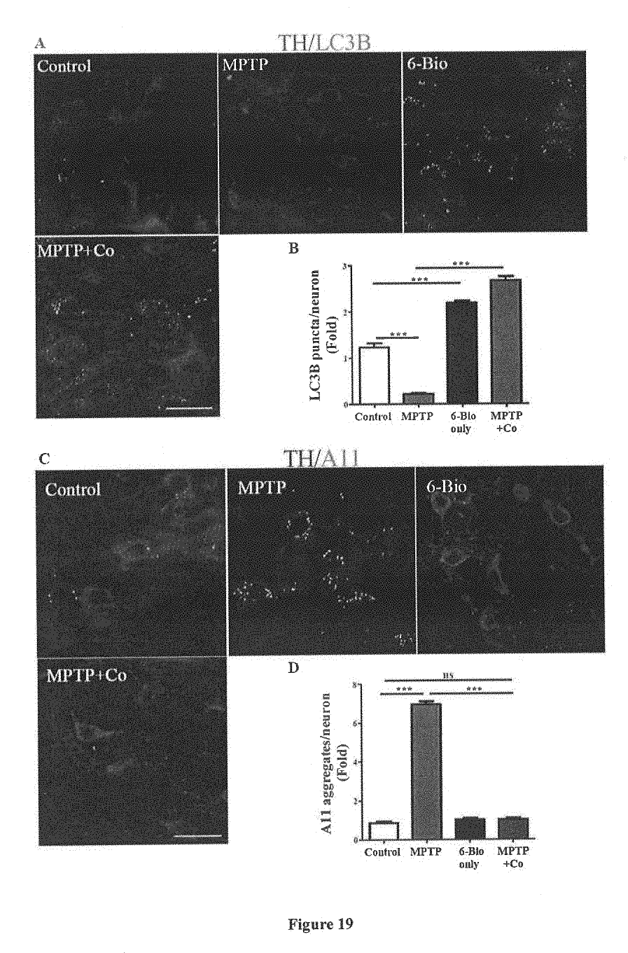

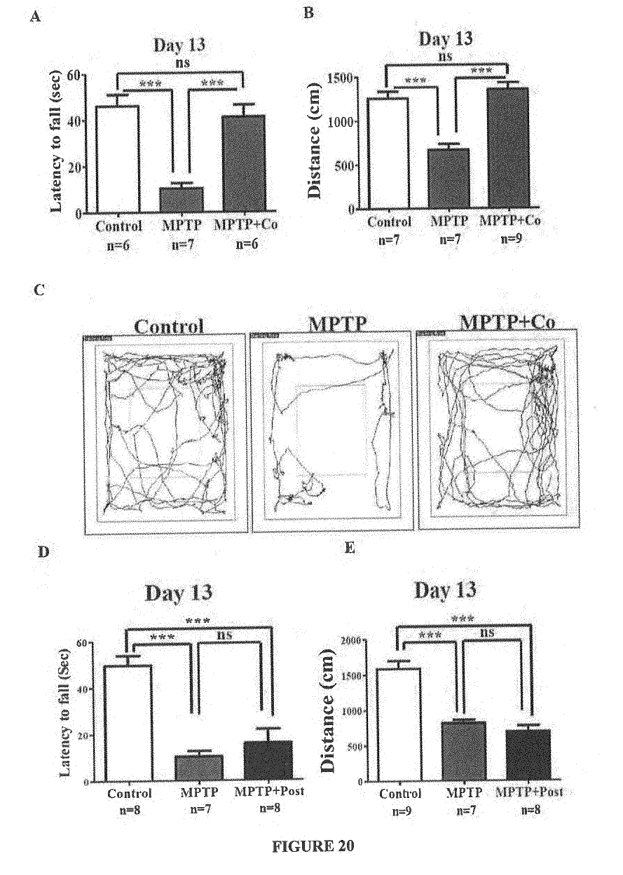

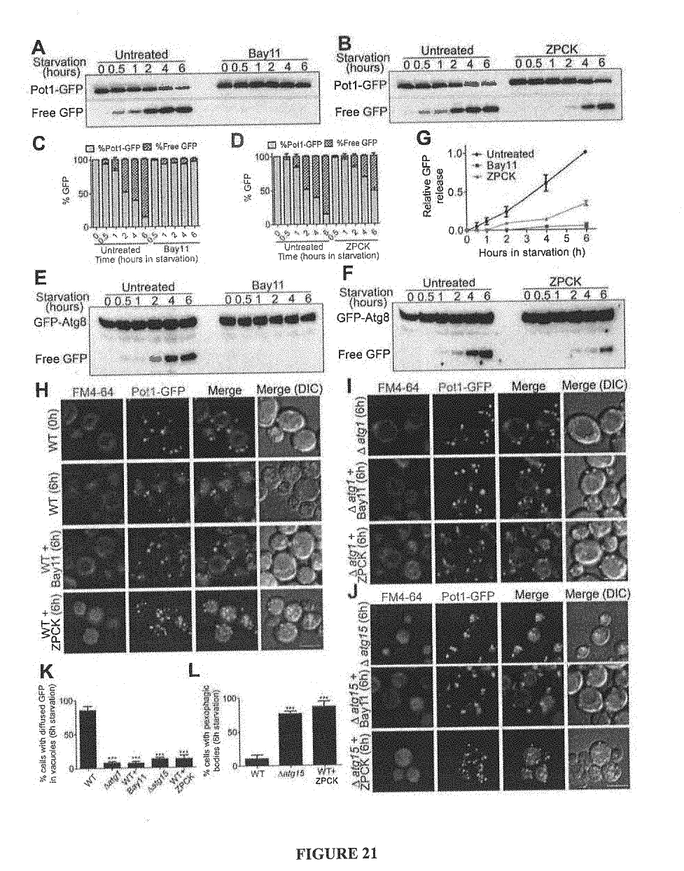

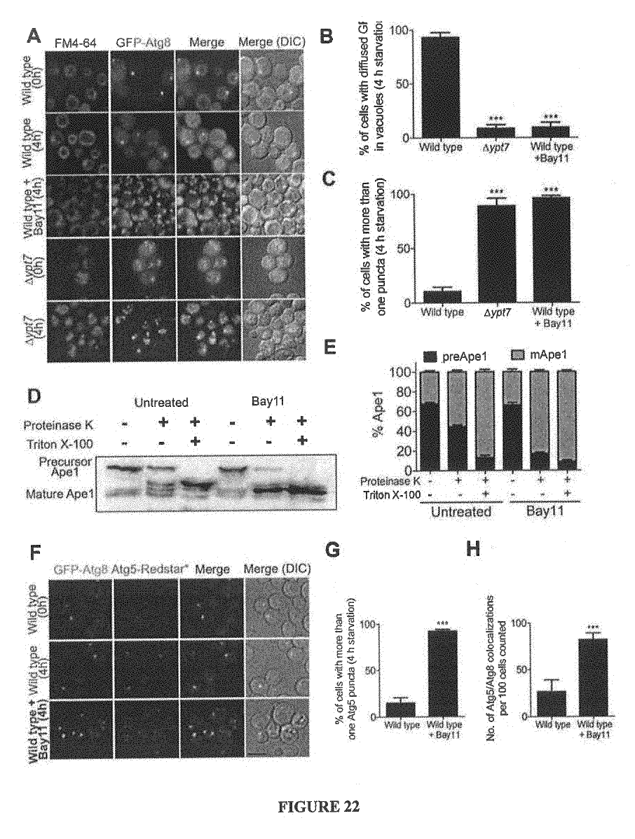

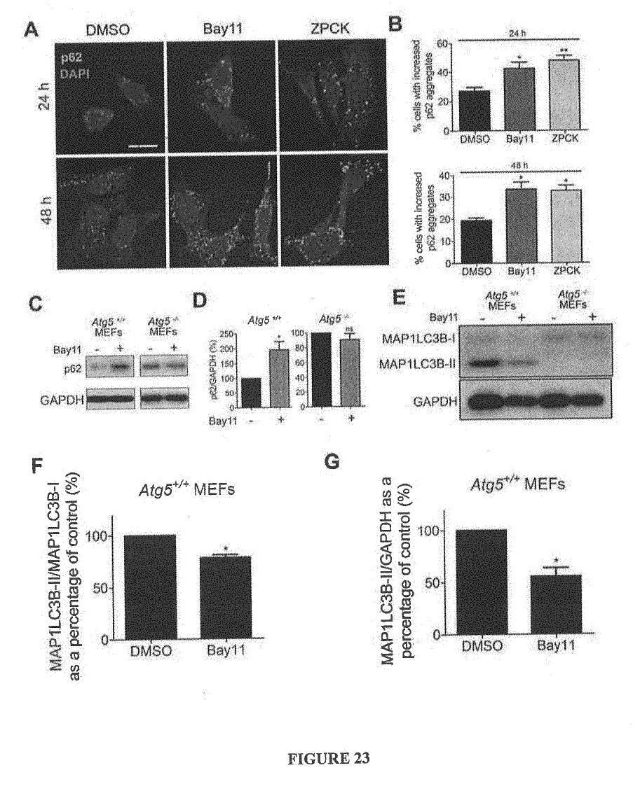

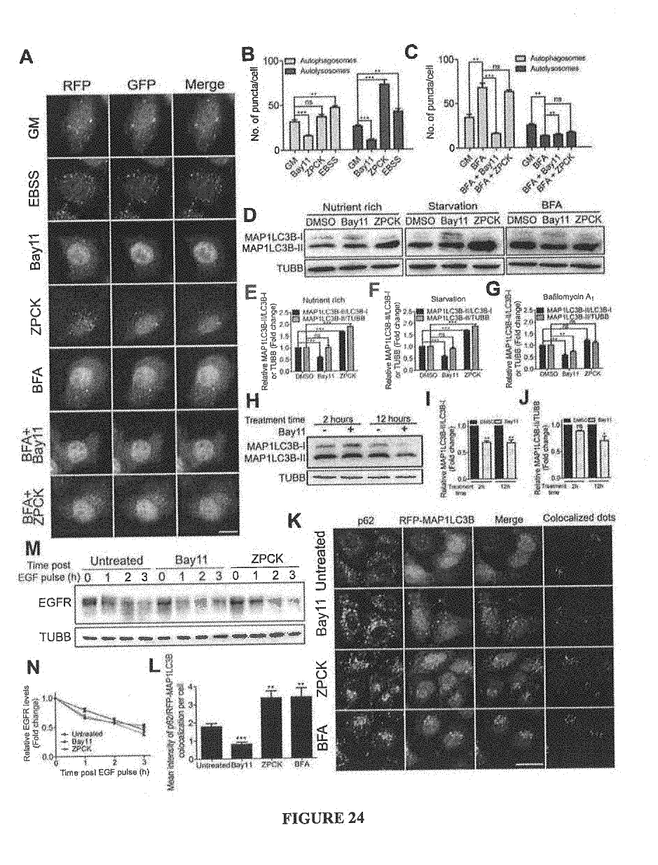

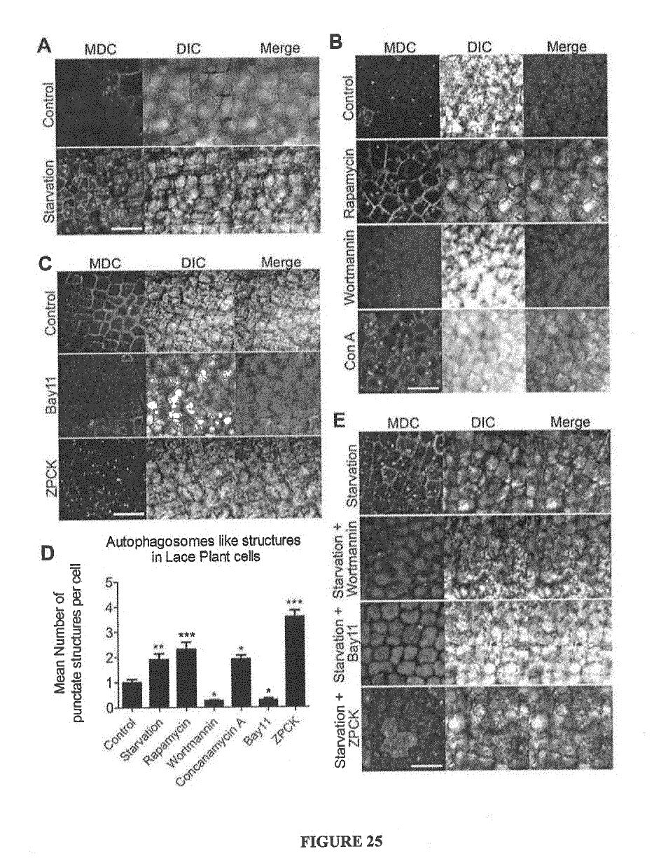

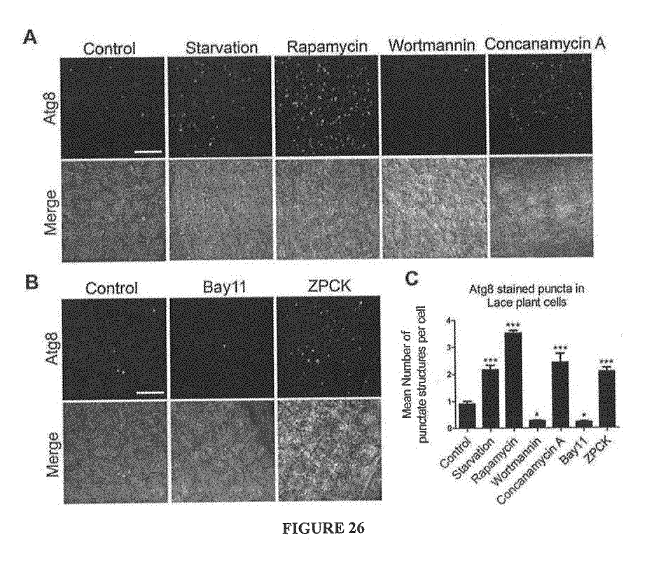

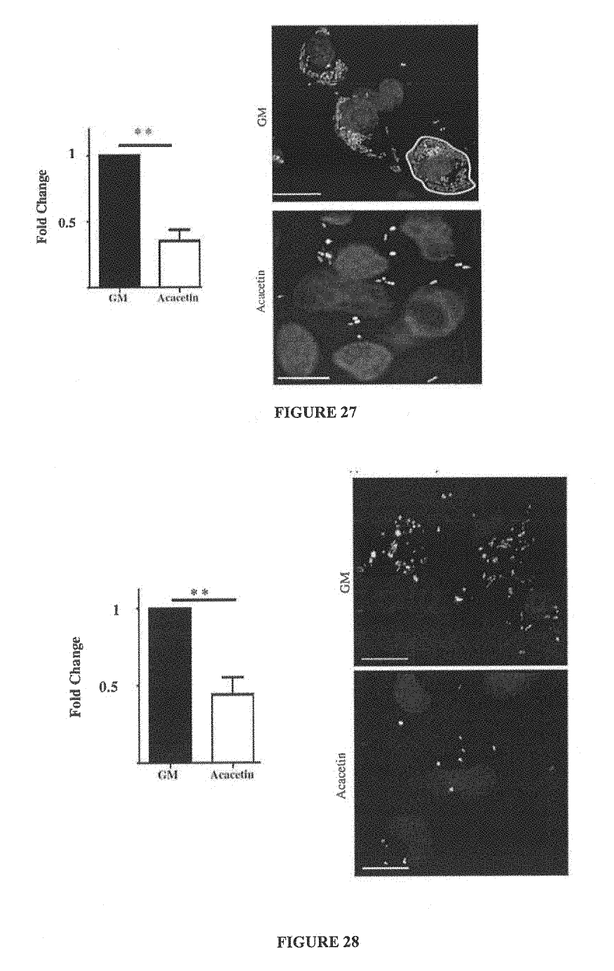

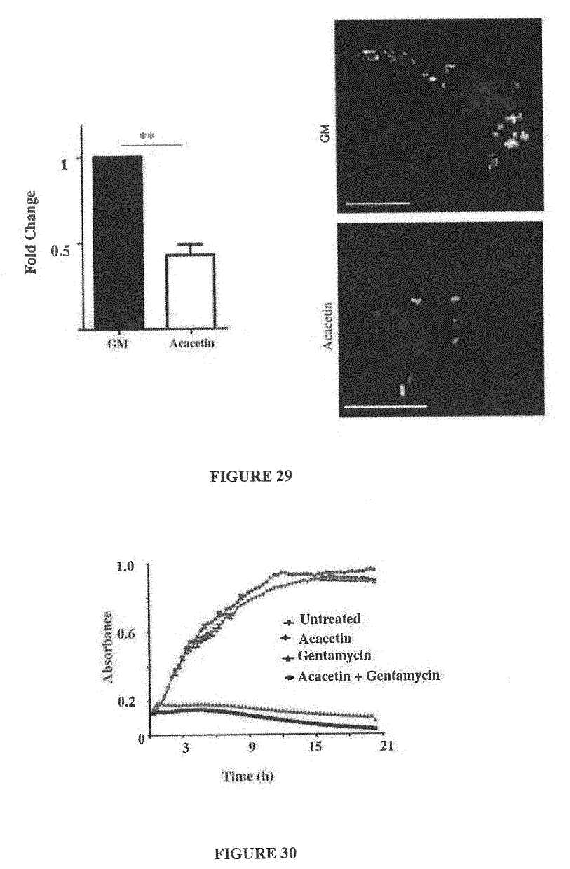

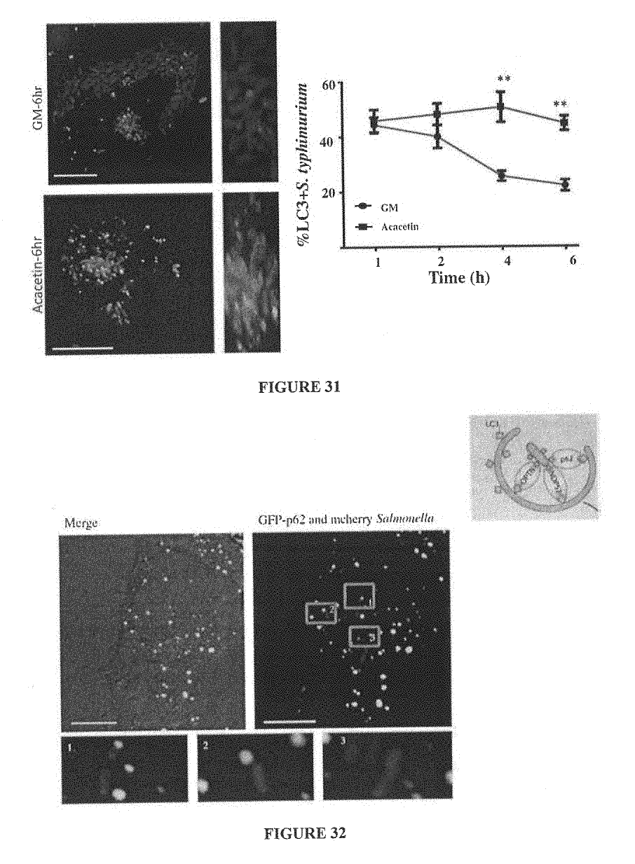

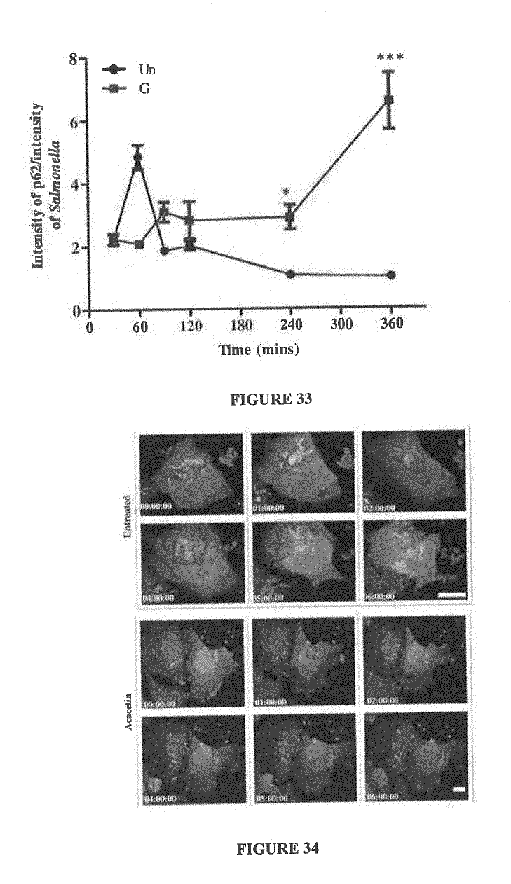

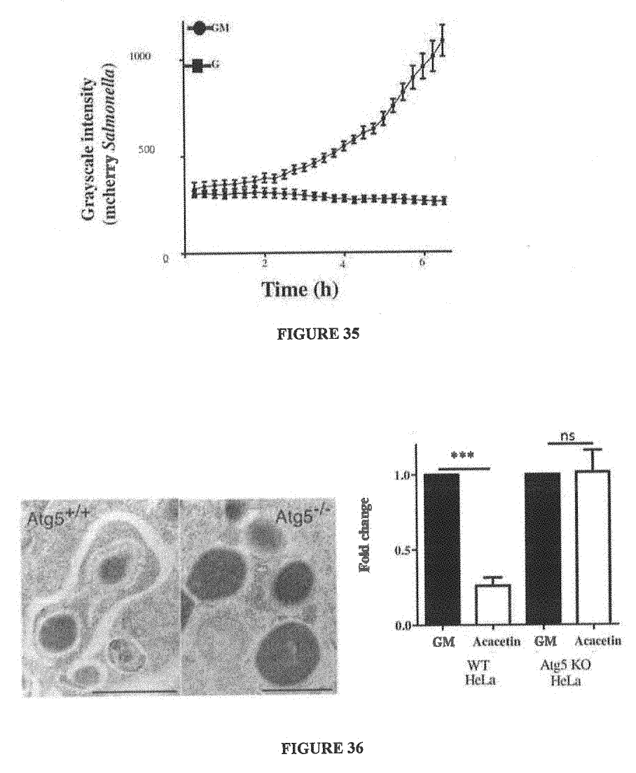

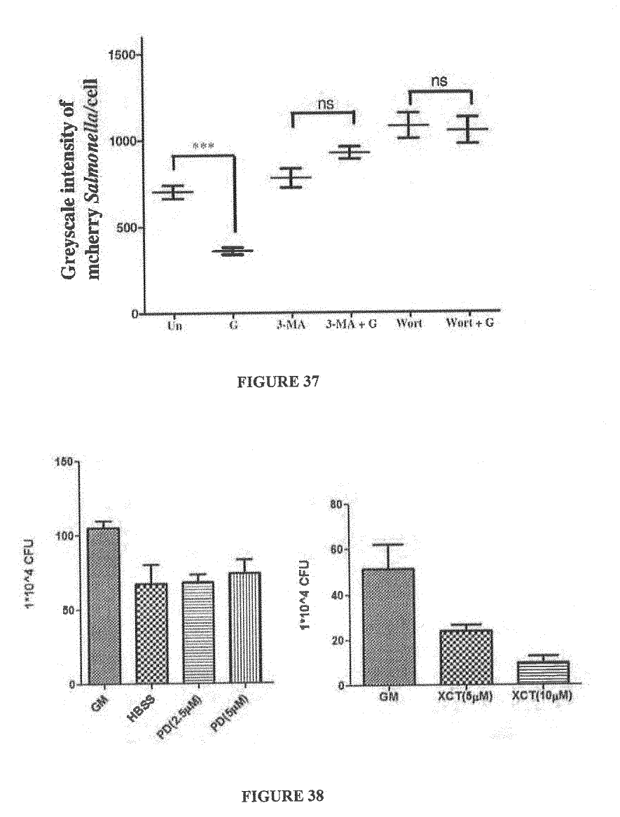

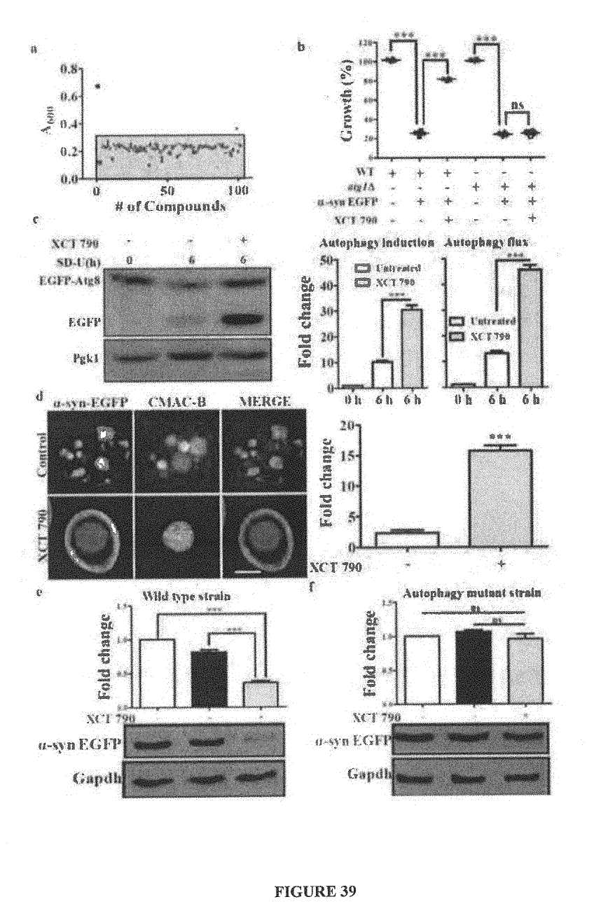

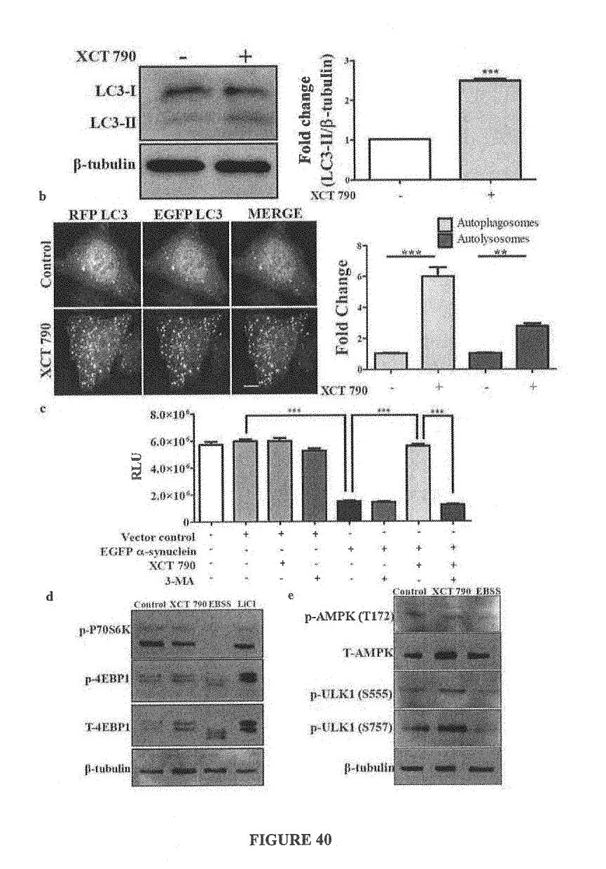

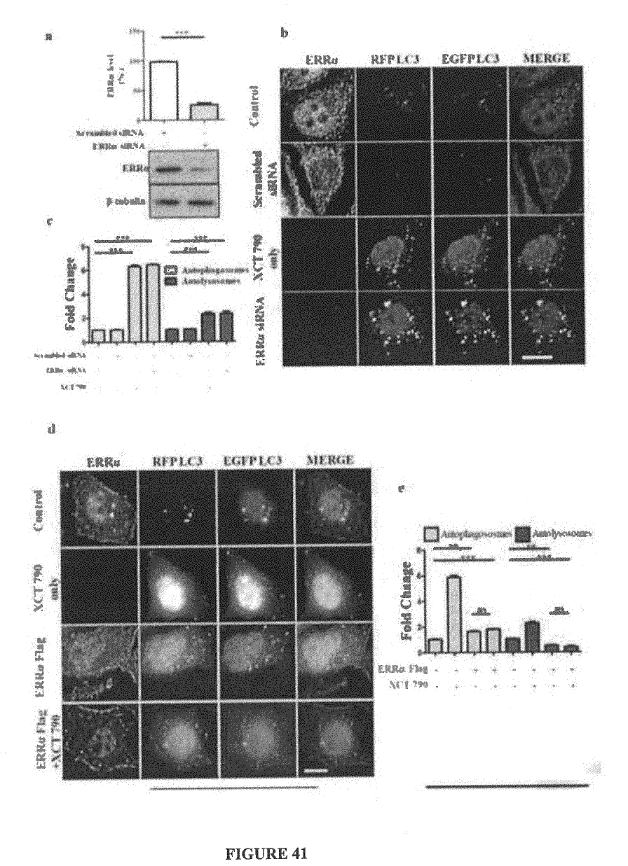

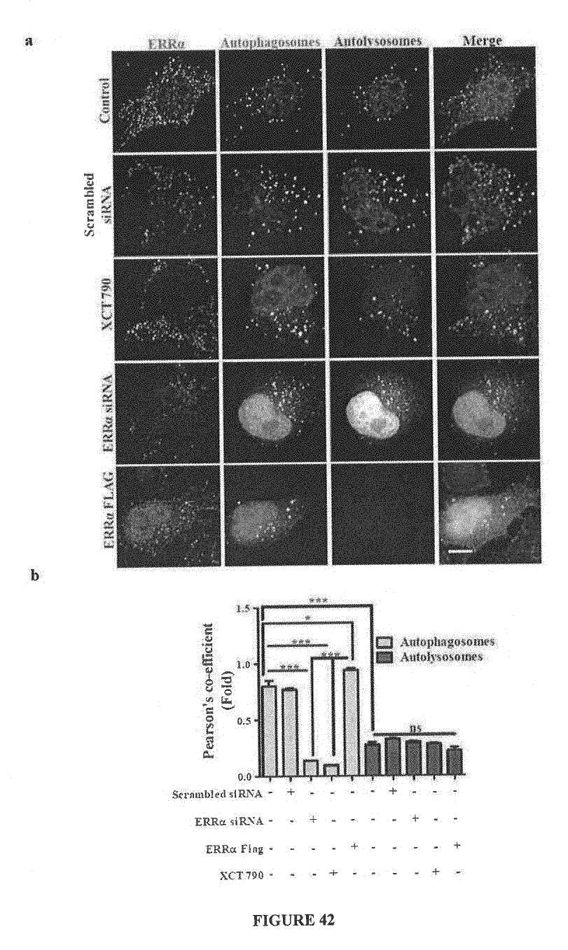

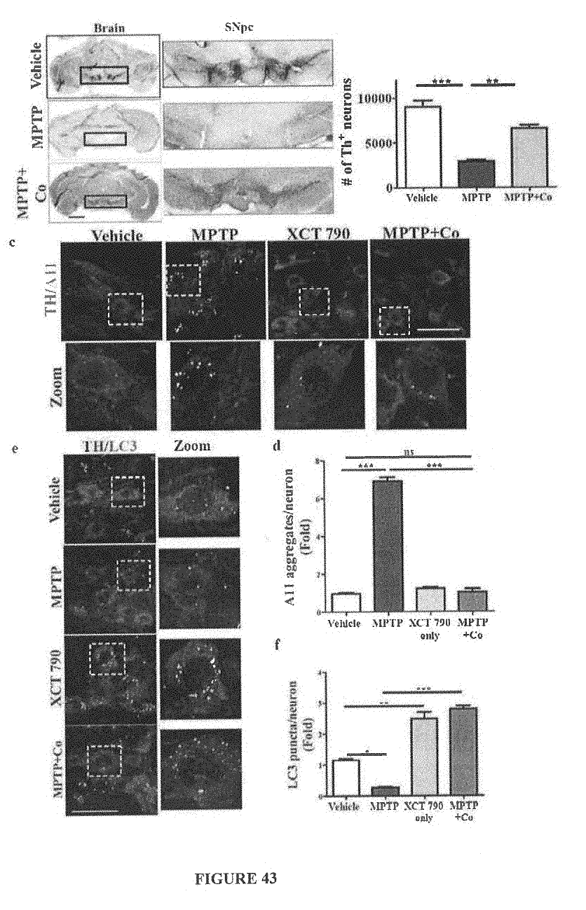

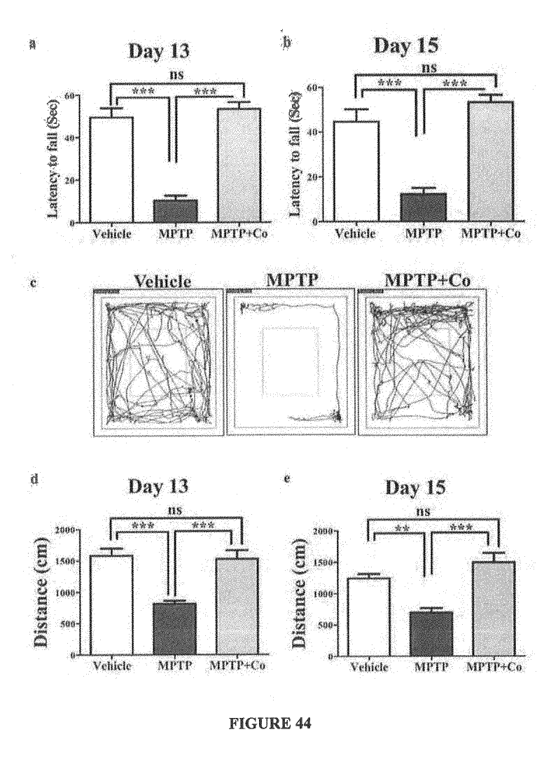

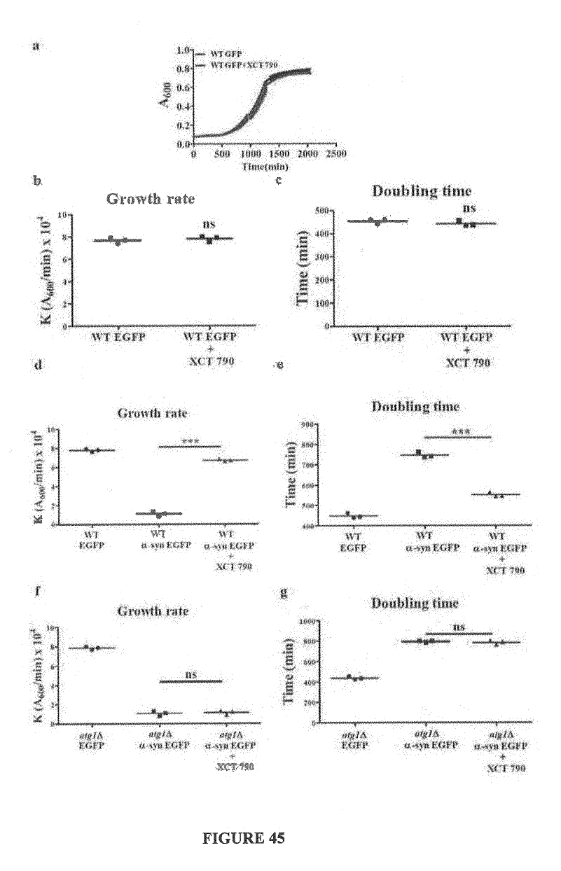

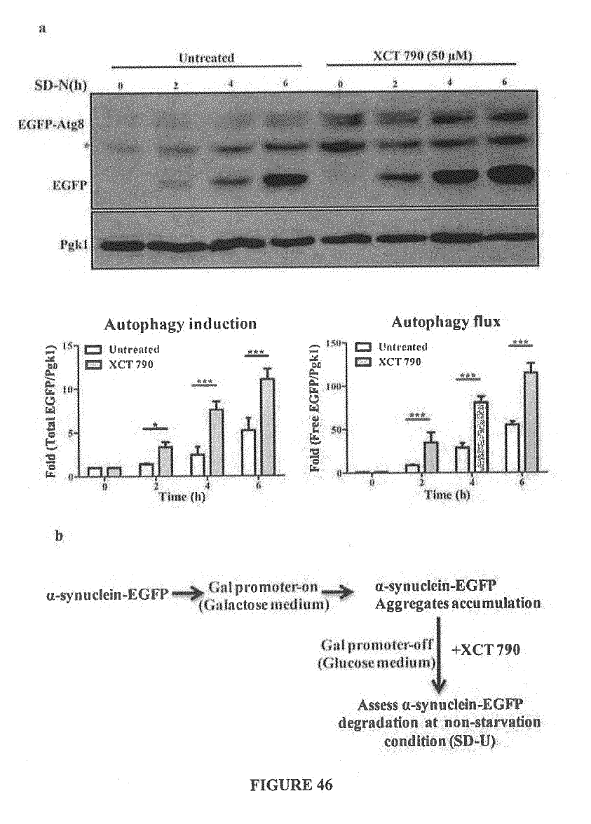

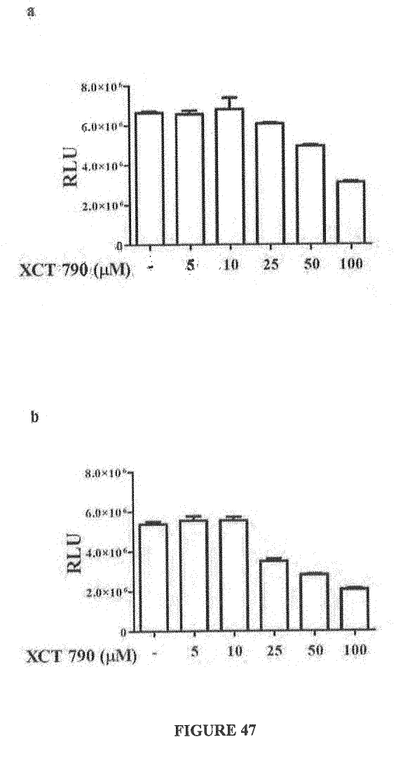

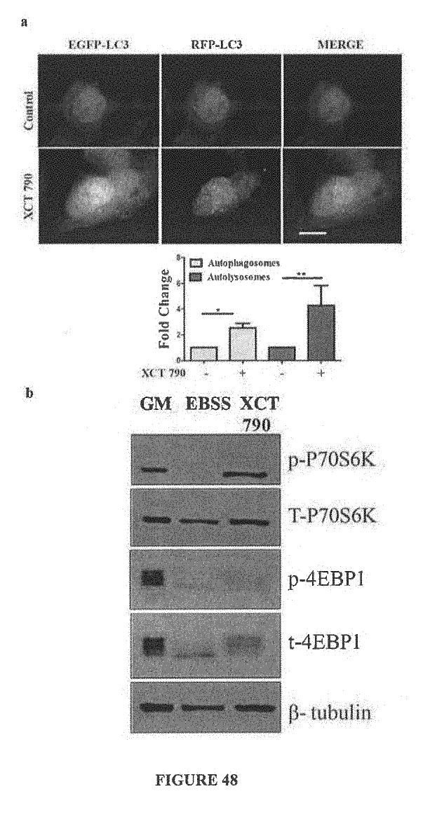

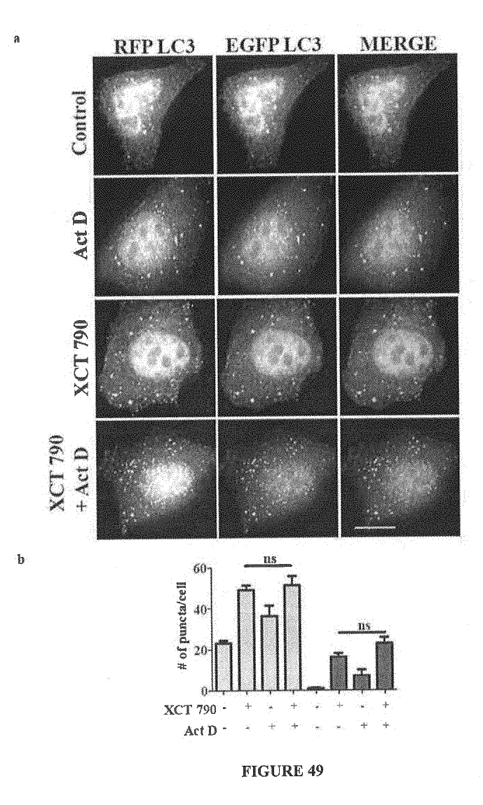

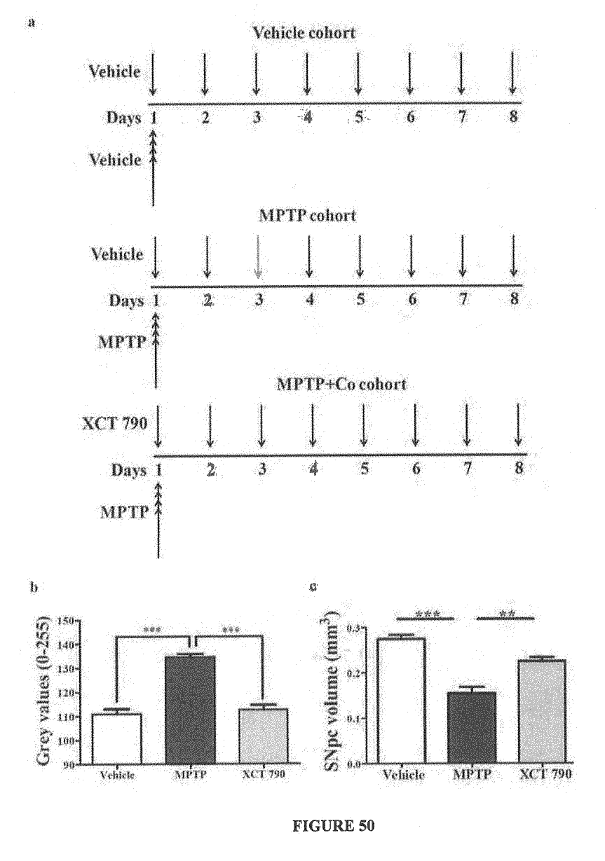

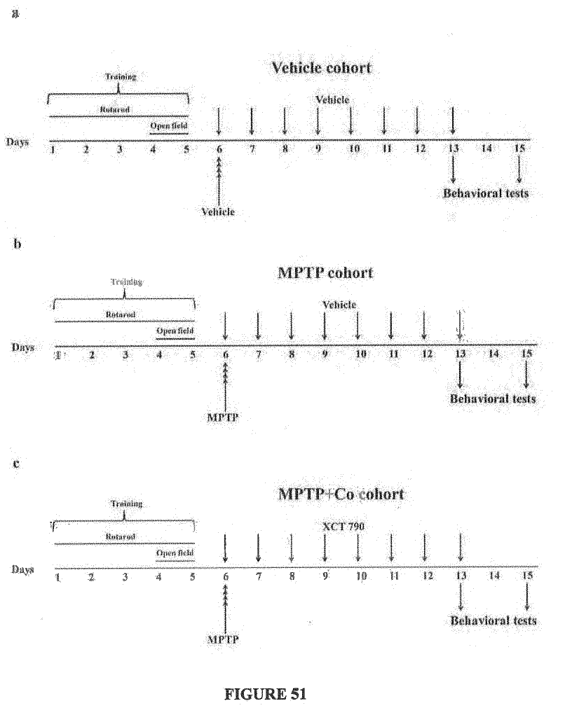

In another embodiment of the present disclosure, FIG. 3 illustrates the effect of 6-Bio on autophagy, wherein (A) box plot representative for 100 compounds demonstrates hits from small molecule library of pharmacologically active compounds, LOPAC1280, screened in S. cerevisiae toxicity model of α-synuclein; wherein compounds that rescue growth lag due to α-synuclein toxicity(denoted by absorbance, A600) of WT α-synuclein-EGFP strains≥3 SD units (grey box) are considered hits (blue) and the ones that do not are in green, while WT EGFP (black) and untreated WT α-synuclein-EGFP (red) represent the positive and negative controls respectively; (B) demonstrates growth curve of WT EGFP cells with and without 6-Bio (50 μM) treatment; (C) represents one-way ANOVA and post-hoc Bonferroni test (Error bars, mean±SEM. ***-P<0.001) which demonstrates representative western blot of GFP-Atg8 processing assay under growth condition; wherein WT strain expressing GFP-Atg8 is treated with 6-Bio (50 μM) and without 6-Bio, respectively, fusion protein GFP-Atg8 accumulation and free GFP release is monitored across time course (0 and 6 h) and GAPDH serves as a loading control; representative graph for quantification of autophagic induction and flux (n=4) is drawn; (D) represents two-way ANOVA and post-hoc Bonferroni test (Error bars, mean±SEM. ns-non significant, *-P<0.05, **-P<0.01, ***-P<0.001) which shows representative western blot for GFP-Atg8 processing assay; wherein fusion protein GFP-Atg8 accumulation and free GFP release are monitored across time course (0, 2 h, 4 h and 6 h) under starvation condition in WT strain expressing GFP-Atg8 treated with 6-Bio (50 μM); representative graph for quantification of autophagic induction and flux (n=3) is drawn; wherein * indicates non-specific band and PGK1 is used as loading control; (E) represents student's unpaired t-test and post-hoc Bonferroni test (Error bars, mean±SEM, **-P<0.01) which shows representative microscopy images of ptf LC3 transfected HeLa cells treated with 6-Bio (5 μM) and quantification of autophagosome and autolysosome indicating fold change over its untreated counterpart (n=25), scale bar 15 μm; wherein statistical analysis is performed using Student's unpaired t-test, two-way ANOVA and post-hoc Bonferroni test. Error bars, mean±SEM, ns-non significant, *-P<0.05, **-P<0.01, ***-P<0.001.

In another embodiment of the present disclosure, FIG. 4 shows that 6-Bio clears α-synuclein in an autophagy dependant manner; wherein (A) represents microscopy images of WT α-synuclein-EGFP yeast cells treated with and without 6-Bio (50 μM) for 16 h, vacuole stained with CMAC-Blue (100 nM), scale bar=2 μm; (B to E) represents two-way ANOVA and post-hoc Bonferroni test (Error bars, mean±SEM. ns-non significant, *-P<0.05, **-P<0.01, ***-P<0.001) which shows quantification plots for α-synuclein-EGFP degradation assay in wild-type (WT) yeast strain for growth (B) and starvation (D) conditions, α-synuclein-EGFP degradation assay in autophagy mutant (atg1α) strain under given growth (C) and starvation (E) conditions; wherein α-synuclein-EGFP protein levels after 6-Bio (50 μM) treatment are analyzed for different time points (0, 6 h, 12 h and 24 h) and GAPDH used as loading control; (F) represents student's unpaired t-test and post-hoc Bonferroni test. Error bars, mean±SEM. ns-non significant, ***-P<0.001 which shows western blot and graph indicating fold change in EGFPα-synuclein degradation over untreated; wherein EGFP-α-synuclein transiently transfected in SH-SY5Y cells are allowed to express for 24 h, treated with 6-Bio (5 μM), 3-MA (5 μM) or both for 24 h post-transfection and assessed for EGFP-α-synuclein degradation; statistical analysis is performed using two-way ANOVA and post-hoc Bonferroni test. Error bars, mean±SEM. ns-non significant, *-P<0,05, **-P<0.01, ***-P<0.001.

In another embodiment of the present disclosure, FIG. 5 demonstrates that 6-Bio enhances mTOR dependant autophagy and confers neuroprotection in a mouse MPTP toxicity model wherein, (A) shows representative western blots indicating dose-dependent modulation of autophagy related proteins (LC3, P70S6 kinase and 4E-BP1) by 6-Bio in HeLa cells; while (B to D) show representative photomicrographs of TH+immunostained dopaminergic neurons (B) in SNpc (arrow) of mouse midbrain in control group, MPTP treated (23 mg/kg body weight), 6-Bio (5 mg/kg body weight) or both [Prophylaxis (MPTP+Pro)/Co-administration (MPTP+Co)], scale bar=300 μm; (C) shows stereological quantification and (D) shows densitometric quantification indicating the number of TH+DA and its intensity in SNpc neurons with both (C) and (D) representing one-way ANOVA and post-hoc Bonferroni test, Error bars, mean±SEM, ns-non significant, ***-P<0.001; statistical analysis is performed using Student's unpaired t-test, one-way/two-way ANOVA and post-hoc Bonferroni test. Error bars, mean±SEM. ns-non significant, **-P<0.01, ***-P<0.001.

In another embodiment of the present disclosure, FIG. 6 demonstrates S. cerevisiae α-synuclein toxicity model; wherein, (A) represents the growth curve, (B) represents growth rate and (C) represents doubling time of WT α-synuclein-EGFP (red curve) versus WT EGFP (black curve) strains with both (B) and (C) showing ***-P<0.001 as determined by one-way ANOVA analysis, post-hoc Bonferroni test. Error bar indicates, mean±SEM,*** -P<0.001 as determined by one-way ANOVA analysis, post-hoc Bonferroni test. Error bar indicates, mean±SEM.

In another embodiment of the present disclosure, FIG. 7 demonstrates scheme for small molecule screening in S. cerevisiae α-synuclein toxicity model; wherein, induction of α-synuclein-EGFP overexpression is carried out in presence and absence of small molecules, respectively from the LOPAC library in duplicates. Growth is recorded as absorbance (A600) and a box plot for each compound is plotted; wherein, the “hits” are compounds that rescue the growth lag due to u-synuclein toxicity (≥3 SD units) and also show free GFP in the vacuole along with restoration of native localization of α-synuclein-EGFP din plasma membrane (B) unlike vehicle treated cells (A); scale bar=2 μm.

In another embodiment of the present disclosure, FIG. 8 shows that 6-Bio fails to rescue growth lag due to α-synuclein toxicity in autophagy mutants expressing α-synuclein; wherein (A) represents statistical analysis performed using one-way ANOVA and post-hoc Bonferroni test (Error bars, mean±SEM. ***-P<0.001) which shows plot indicating the percent growth of WT α-synuclein-EGFP strain in presence of Agk2 (50 μM) and 6-Bio (50 μM), (B) shows growth of autophagy mutants (atg1Δ, atg5Δ, atg8Δ, atg11Δ and atg15Δ) expressing α-synuclein-EGFP observed with or without 6-Bio (50 μM); statistical analysis was performed using one-way ANOVA and post-hoc Bonferroni test. Error bars, mean±SEM, ***-P<0.001.

In another embodiment of the present disclosure FIG. 9 demonstrates α-synuclein-EGFP degradation assays in yeast; wherein, (A) shows schematic representation of α-synuclein-EGFP degradation assay conditions; (B to E) show western blots for α-synuclein-EGFP degradation upon 6-Bio (50 μM) administration, in WT and autophagy mutant (atg1Δ) cells under growth (B and D) and starvation (C and E) conditions; wherein, GAPDH or PGK1 are used as loading control. SD-U is growth medium while SD-N is nitrogen starvation medium.

In another embodiment of the present disclosure, FIG. 10 shows 6-Bio administration in MPTP mouse model; wherein, (A) shows scheme representing schedule of dosage administration of MPTP (23 mg/kg), 6-Bio (5 mg/kg) in mice groups; (B) shows representative photomicrographs of TH+ immunostained DA neurons in SNpc of mouse midbrain of control, MPTP and 6-Bio and both [Prophylaxis (MPTP+Pro)/Co-administration (MPTP+Co)] groups and (C) represents statistical analysis performed using one-way ANOVA and post-hoc Bonferroni test (Error bars, mean±SEM. **-P<0.01, ***-P<0.001) which shows quantitative plot of SNpc volume of mouse brains for all the groups; Statistical analysis is performed using one-way ANOVA and post-hoc Bonferroni test. Error bars, mean±SEM. **-P<0.01, ***-P<0.001.

In another embodiment of the present disclosure, FIG. 11 represents Pot1 GFP assay for Acacetin; wherein Pot1-GFP positive strains are allowed to grow till the Absorbance at 600 nm reaches 0.6-0.8 in YPD; peroxisome biogenesis is induced by growing these cells in YPG medium (1% yeast extract, 2% peptone, 3% glycerol) for 12 hours; cells are harvested, washed twice to remove traces of oleate and transferred to starvation medium with and without Acacetin, at inoculum density Absorbance, at 600 nm 3/ml, to induce pexophagy; cells are collected at various time intervals after pexophagy induction and processed by TCA method; wherein, cells treated with Acacetin show an enhanced accumulation of free GFP over time as compared to the untreated cells, indicating an to increase in the levels of pexophagy.

In another embodiment of the present disclosure, FIG. 12 represents statistical analysis performed using one-way ANOVA and post-hoc Bonferroni test (Error bars, mean±SEM. *-P<0.05) which demonstrates fold change in CFU for Acacetin using Burden assay; wherein, U1752 cell line (a) and HeLa cell line (b) are infected with Salmonella typhimurium SL1344 then treated with Acacetin and incubated for 3-4 hours; wherein, at the end, the cells are lysed using lysis buffer (0.1% SDS, 1% Triton X-100, 1X PBS) and the intercellular Salmonella is plated and the CFU is counted; cells are collected at various time intervals after pexophagy induction and processed by TCA method; wherein, cells treated with Acacetin show an enhanced accumulation of free GFP over time as compared to the untreated cells, indicating an increase in the levels of pexophagy; CFU of Acacetin treated is reduced about 1.8 fold to about 2.5 fold compared to that of untreated; Statistics done using Graphpad prism—two tailed T test.

In another embodiment of the present disclosure, FIG. 13 shows growth curve of Salmonella typhimurium SL1344; wherein, a single colony of Salmonella typhimurium WT strain SL1344 grown overnight at 37° C. is diluted in Luria Broth media to get an O.D of 0.2 and the diluted culture is used for treatments with Acacetin and Acacetin with gentamycin (100 μg/ml); growth curve of the culture is obtained by measuring the absorbance at 600 nm using varioskan Flash Multiplate Spectrophotometer at 300 rpm and O.D is taken at every 30 minutes interval for 10 hours is plotted using GraphPad Prism; it is observed that Acacetin does not have any anti-microbial activity against Salmonella typhimurium SL1344.

In another embodiment of the present disclosure, FIG. 14 shows co-localization GFP-LC3 with mcherry Salmonella typhimurium SL1344; wherein, a) HeLa cells are transfected with GFP-LC3 using lipofectamine 3000, after 24 hours, cells are infected with Salmonella typhimurium WT strain SL1344 with an MOI of 400 for 15 minutes followed by gentamycin treatment at the concentration of 100 μg/ml for 10 minutes to kill the extracellular bacteria; the cells are treated with Acacetin and without Acacetin, respectively and incubated for different time points at 37° C.; and b) quantitation of LC3 co-localization with Salmonella typhimurium SL1344 is done using ImageJ-Cell counter option.

In another embodiment of the present disclosure, FIG. 15 represents Live Cell Microscopy images; wherein, GFP-LC3 transfected HeLa cells are infected with mcherry-Salmonella typhimurium SL1344 for 15 minutes (MOI-400) and are treated with gentamycin for 10 minutes; the cells are then washed with 1X PBS and changed to either only media (a) or media containing Acacetin (b) and imaged by FV10i-olympus confocal live cell imaging microscope, using 60x water immersion lens, with confocalityaperature set to 1.0; images are taken at an interval of 15 minutes; wherein (c) intensity of the Red channel is measured using image J-Stacks T function; it is observed that replication of Salmonella in Acacetin treated samples is restricted as compared to that of untreated samples.

In another embodiment of the present disclosure, FIG. 16 shows that Traffic Light Assay for Acacetin; wherein, a) ptf-LC3 transfected HeLa cells are treated with the Acacetin for 2 hours; b) number of autophagosomes and autolysosomes are counted using imageJ-cell counter function, wherein, starvation medium (HBSS), is used as positive control which shows higher counts than the basal level of growth medium (GM) and the compound treated sample shows an increase in the number of autolysosomes (red dots).

In another embodiment of the present disclosure, FIG. 17 demonstrates that Bay11-7082 blocks initial step of autophagy whereas ZPCK acts towards the later stages; a) shows POT1-GFP processing assay for accessing the effect of Bay11-7082 and ZPCK on pexophagy; wherein no free GFP release is seen on treatment of wild type cells with Bay11-7082 even after 6 hours of starvation, whereas very little free GFP is observed only at the later time points in ZPCK treated cells; b) effect of the inhibitors on general autophagy is monitored by GFP-Atg8 assay; wherein, no release of GFP is observed on treatment with either Bay11-7082 or ZPCK as compared to the untreated cells; c) indicates pexophagy as monitored via fluorescence microscopy reveals that Bay11-7082 acts at a step prior to fusion of autophagosomes with the vacuole (labelled with FM4-64); d) shows the protease protection assay wherein, conversion of precursor to matured form of aminopeptidase on treatment with proteinase K in Bay11-7082 treated cells indicates that the cargo is not protected by the autophagosome membrane and thus autophagosome biogenesis may be inhibited on treatment with Bay11-7082.

In another embodiment of the present disclosure, FIG. 18 shows Bay11-7082 and ZPCK inhibit autophagy in HeLa cells; wherein, (a) Hela cells transfected with ptf-LC3 (vector having tandem mRFP-GFP tagged LC3) treated with Bay11-7082 and ZPCK for 2 hours in growth medium are observed under fluorescence microscope; wherein, autophagosomes appear as yellow dots whereas autolysosomes appear red inside the cells; it is observed that on treatment with ZPCK, autolysosomes increases inside the cells, which is in accordance with the earlier observation made in yeast cells. On Bay11-7082 treatment, very few autophagosomes are seen, confirming that Bay11-7082 blocks the formation of autophagosomes.

In another embodiment of the present disclosure, FIG. 19 shows induction of autophagy in mice brain by 6-Bio to clear toxic protein aggregates, wherein (A) Representative immuno histofluorescent photomicrographs of various cohorts namely control, MPTP (23 mg/kg of body weight), 6-Bio (5 mg/kg of body weight) and MPTP+Co that were stained for LC3B (an autophagy marker) and TH (SNpc) in midbrain. Autophagic modulation by 6-Bio were evaluated in DAergic neurons in SNpc and the LC3B puncta fold change per neuron was quantitated (B). (C) Representative immuno histofluorescent photomicrographs of above mentioned cohorts were stained for A11 (toxic oligomers) and TH (SNpc) in midbrain. Aggregate clearance by 6-Bio were evaluated in DAergic neurons in SNpc and the A11 puncta fold change per neuron was quantitated (D). Statistical analysis was performed using one-way ANOVA and post-hoc Bonferroni test. Scale bar=50 μm. Error bars, mean±SEM. ns-non significant, ***-P<0.001.

In another embodiment of the present disclosure, FIG. 20 shows amelioration of MPTP-induced behavioral deficits by 6-Bio, wherein (A) latency to fall of various cohorts namely Placebo, MPTP and MPTP+Co as assessed by rotarod test (B) Representative trajectory maps of all mentioned cohorts as analyzed by open field test. (C) Periphery distance travelled by all indicated cohorts as assessed by open field test. Effect of 6-Bio (5 mg/kg) on various cohorts namely Placebo, MPTP and MPTP+Post. (D) latency to fall of various cohorts namely Placebo, MPTP and MPTP+Post as assessed by rotarod test. (E) Periphery distance travelled by all indicated cohorts as assessed by open field test. Both rotarod and open field behavior analyses performed on day 13 or day 7 post-MPTP/vehicle administrations. Both rotarod and open field behavior analyses performed on day 13 or day 7 post-MPTP/vehicle administrations. 6-Bio (5 mg/kg) was administrated either along with MPTP (MPTP+Co) or post 48 h of MPTP administration (MPTP+Post). Statistical analysis was performed using one-way ANOVA and post-hoc Bonferroni test. Scale bar=50 μm. Error bars mean±SEM. ns-non significant, ***-P<0.001.

In another embodiment of the present disclosure, FIG. 21 illustrates blockage of initial step of autophagy by Bay 11 whereas ZPCK acts towards the later stages of autophagy in yeast Saccharomyces cerevisiae, wherein (A) Pot1-GFP processing assay for assessing the effect of Bay11 and (B) ZPCK on pexophagy. No free GFP release was seen on treatment of wild type cells with Bay11 even after 6 hours of starvation, whereas very little free GFP was observed only at the later time points in ZPCK treated cells as quantified in (C) and (D). Effect of Bay11 (E) and ZPCK (F) on general autophagy was monitored by GFP18 Atg8 assay. No or delayed release of GFP was observed on treatment with either Bay11 or ZPCK respectively as compared to the untreated cells (G). Pexophagy (degradation of peroxisomes via autophagy) as monitored via fluorescence microscopy revealed that Bay11 acted at a step prior to fusion of autophagosomes with the vacuole (H) (labelled with FM4-64). No free GFP was seen inside the vacuole and the peroxisomes were present in the cytosol even on autophagy induction, morphology similar to an early step mutant of autophagy Δatg (H), (I) and (J). On treatment with ZPCK, peroxisomes got accumulated inside the vacuole, morphology similar to Δatg15, an autophagy mutant deficient in the vacuolar proteases (H), (I) and (J). (K) Quantitation showing percentage number of cells with diffused GFP accumulation inside the vacuole in different treatment conditions after 6 hours in starvation. (L) Graph showing percentage number of cells with accumulation of pexophagic bodies inside the vacuole on starvation in wild type, Δatg15 and wild type cells treated with ZPCK.

In another embodiment of the present disclosure, figure illustrates effect of Bay11 treatment on maturation of autophagosomes, wherein (A) GFP-Atg8 fluorescence microscopy showed an accumulation of GFP-Atg8 positive puncta on treatment with Bay11 under starvation condition. Graphs showing diffused GFP inside the vacuole (B) and number of puncta in the cytosol at 4 hours of starvation (C) in wild type, Δypt7 and wild type cells treated with Bay11. (D) To elucidate the step of action of Bay11, a protease protection assay was performed using aminopeptidase as a marker, which is also a substrate for autophagy on starvation. Conversion of precursor to matured form of aminopeptidase on treatment with proteinase K (PK) in Bay11 treated cells indicated that the cargo is not protected by the autophagosome. (E) Quantitation showing relative precursor and mature form of aminopeptidase levels for different treatment groups. Y-axis shows the total aminopeptidase levels. TX—Triton X-100; PK—Proteinase K, (F) Co-localization of genomically tagged GFP-Atg8 and Atg5-RedStar* proteins in untreated and Bay11 treated conditions. (G) Quantitation showing percentage number of cells with more than one Atg5 puncta. (H) Quantitation showing number of co-localization events per 100 cells in untreated and Bay11 treated cells. Scale bar=5 μm. Data shown represent a minimum of 100 cells from 3 independent experiments and are expressed as the mean±SD. ***P<0.001 (individual means compared using two-tailed unpaired t-test).

In another embodiment of the present disclosure, FIG. 23 illustrates inhibition of autophagy by Bay11 and ZPCK in MEFs, wherein (A and B) MEFs were treated with DMSO (vehicle control). 5 μM Bay11 or 5 μM ZPCK for 24 h or 48 h, fixed for immunofluorescence analysis with anti-p62 antibody and imaged by confocal microscopy (A). Analysis was done for the percentage of cells with accumulated endogenous p62+ aggregates (B). Scale bar, 20 μm. (C and D) Atg5+/+ (wild-type) and Atg5−/− (autophagy6 deficient) MEFs were treated with DMSO (vehicle control) or 5 μM Bay11 for 24 h, followed by immunoblotting analysis with anti-p62 and anti-GAPDH antibodies. Densitometric analysis of p62 levels was done relative to GAPDH where the control (DMSO9 treated) condition was fixed at 100%. (E) Atg5+/+ and Atg5−/− MEFs were treated with DMSO (vehicle control) or 5 μM. Bay11 for 24 h, followed by immunoblotting analysis with anti-MAP1LC3B and anti-GAPDH antibodies. (F) MAP1LC3B-II/MAP1LC3B-I and (G) MAP1LC3B-II/GAPDH levels quantitated for 3 independent experiments in DMSO and Bay11 treated cells.

In another embodiment of the present disclosure, FIG. 24 illustrates inhibition of autophagy by Bay11 and ZPCK in HeLa cells at different stages, wherein (A) Hela cells transfected with ptf-MAP1LC3B (vector having tandem mRFP-GFP tagged MAP1LC3B) treated with either Bay11 or ZPCK for 2 hours in growth medium in the presence or absence of Bafilomycin A1 (400 nM) were observed under fluorescence microscope. Autophagosomes appear as yellow dots whereas autolysosomes appear red inside the cells. On treatment with ZPCK, autolysosomes increased inside the cells whereas on Bay11 treatment, very few autophagosomes were seem Scale bar=15 μM (B and C) Data shown represent a minimum of 65 cells from 3 independent experiments with number of autophagosomes and autolysosomes counted and are expressed as the mean±SD, ***P<0.001 (One way ANOVA, individual means compared with a Dunnett's Multiple Comparison Test) (D) MAP1LC3B conversion assay for the mentioned treatment groups under nutrient rich, starvation conditions and along with bafilomycin A1. (E-G) MAP1LC3B2 II/MAP1 LC3B-I and MAP1LC3B-II/TUBB levels were quantified for all conditions and plotted. (H) MAP1LC3B conversion assay in the absence and presence of Bay11 for 2 and 12 hours. (I) MAP1LC3B-II/MAP1LC3B-I and (J) MAP1LC3B-II/TUBB levels of control and Bay11 treated cells over a time course for 3 independent experiments. (K) Immunostaining with p62 antibody in RFP-MAP1LC3B transfected HeLa cells with co-localization. Scale bar=20 μM (L) Graph showing the amount of Co-localization between p62 and RFP8 MAP1LC3B in different treatment groups. The mean intensity of colocalized dots was calculated using Co-localization plug-in of Image J analysis software. (M) EGFR trafficking shown by immunoblot for the mentioned treatment groups and degradation levels quantified (N). Data shown represent a minimum of 65 cells from 3 independent experiments and are expressed as the mean±SD. ***, p<0.001; **, p<0.01; *, p<0.05; ns, non-significant (individual means compared by two-tailed unpaired t-test).

In another embodiment of the present disclosure, FIG. 25 illustrates effect of autophagy modulators in lace plant (Aponogeton madagascariensis) cells. Lace plant leaves treated with different modulators were sectioned and stained using monodansylcadaverine (MDC) and scanned via confocal microscopy with 405/450±35 nm (ex/em). (A) There were significantly more punctate structures (autophagosome like structures) in overnight starvation treatment compared to the control. Scale bar=20 μm (B) The 1 μM concanamycin A and 5 μM rapamycin had a significantly higher number of puncta compared to control, which had more than the 5 μM wortmannin treatment. (C) 50 μM Bay11 significantly reduced, whereas 50 μM ZPCK increased puncta compared to the control. (D) Quantitation of the mean number of punctate structures for each treatment (E) Treatment with 50 μM Bay11 or 5 μM wortmannin of overnight starvation leaves showed fewer puncta as compared to the control. Punctate structures were significantly higher than the control in the Starvation, and 50 μM ZPCK treatments. Data shown represent a minimum of 4 independent experiments and are expressed as the mean±SEM. (One way ANOVA, Dunnett's multiple comparison test (***, p<0.001; **, p<0.01; s, p<0.05). Scale bar: 30 μm.

In another embodiment of the present disclosure, FIG. 26 illustrates immunolocalization of Atg8 in lace plant (Aponogeton madagascariensis) cells. Lace plant leaf pieces treated with modulators revealed similar results to the MDC staining. (A) The starvation, 5 μM rapamycin and 1 μM concanamycin A treatment groups contained more puncta than the control, while the 5 μM wortmannin treatment reduced puncta. (B) 50 μM Bay11 reduced the number of puncta and 50 μM ZPCK increased puncta compared to the control group. (C) Quantitation was done for a minimum of 4 independent replicates per experimental group and statistical significance was calculated (One way ANOVA, Dunnett's multiple comparison test (***, p<0.001; **, p<0.01; p<0.05). Scale bar; 30 μm.

In another embodiment of the present disclosure, FIG. 27 illustrates decrease in intracellular Salmonella typhimurium by Acacetin.

In another embodiment of the present disclosure, FIGS. 28 and 29 illustrates decrease in intracellular Salmonella typhimurium by Acacetin.

In another embodiment of the present disclosure, FIG. 30 illustrates that Acacetin does not have direct anti-bacterial effect.

In another embodiment of the present disclosure, FIG. 31 illustrates that Acacetin increases temporal recruitment of LC3 to mcherry Salmonella typhimurium.

In another embodiment of the present disclosure, FIG. 32 illustrates recruitment of p62 to mcherry Salmonella typhimurium.

In another embodiment of the present disclosure, FIG. 33 illustrates increased temporal recruitment of p62 to mcherry Salmonella typhimurium by Acacetin.

In another embodiment of the present disclosure, FIG. 34 illustrates live cell imaging of Acacetin treated cells.

In another embodiment of the present disclosure, FIG. 35 illustrates arrest of replication of Salmonella in presence of Acacetin.

In another embodiment of the present disclosure, FIG. 36 illustrates non-functionality of Acacetin in Atg5 KO HeLa cell line.

In another embodiment of the present disclosure, FIG. 37 illustrates non-functionality of Compound G in presence of autophagy inhibitors.

In another embodiment of the present disclosure, FIG. 38 illustrates results of burden assay for screening of compounds.

In another embodiment of the present disclosure, FIG. 39 illustrates XCT 790 is a potent autophagy inducer and protects α-synuclein toxicity by clearing them in autophagy dependent manner in yeast, wherein (a) Representative box plot indicating chemical hits attained from small molecule library screened in α-synuclein toxicity model of S. cerevisiae. In the box plot, small molecules that rescued the growth (absorbance, A600) of wild-type (WT) α-synuclein-EGFP strain by ≥3 SD units (grey box) are considered as hits (blue) and that do not rescue the growth are labeled in green, WT EGFP (black) and untreated WT α-synuclein-EGFP (red) strains represent the positive and negative controls of the screen. (b) Percent growth of yeast strains (WI' EGFP, WT α-syn-EGFP, atg1Δ EGFP, atg1Δ αt-syn-EGFP) treated with XCT 790 (n=4, three independent experiments). (c) Representative western blot of GFP Atg8 processing assay and assessed the GFP-Atg8 processing after 6 h of incubation in growth condition treated with XCT 790. Fold change in autophagy induction (EGFP-Atg8 band intensity normalized by loading control) and its flux (summation of EGFP-Atg8 and free EGFP normalized by loading control) modulated by XCT 790 were quantified (three independent experiments). PGK1 served as a loading control. (d) Microscopy images of WT α-synuclein-EGFP treated with XCT 790 and then quantified for the vacuolar free EGFP in fold (n=75 cells). Scale bar 5 μm. (e) Representative western blot for α-synuclein-EGFP degradation in WT α-synuclein-EGFP strain analyzed after 24 h of XCT 790 treatment and quantified for the levels of α-synuclein-EGFP (three independent experiments). Gapdh served as a loading control. (f) Representative western blot for α-synuclein-EGFP degradation in atg1Δ α-synuclein-EGFP strain analyzed after 24 h of XCT 790 treatment and quantified for the levels of α-synuclein-EGFP (three independent experiments). Gapdh served as a loading control. Concentration of XCT 790 used was 50 μM. Statistical analysis was performed using one-way ANOVA and post-hoc Bonferroni test. Error bars, mean±SEM. ns-non significant, ***-P<0.001.

In another embodiment of the present disclosure, FIG. 40 illustrates exertion of cellular neuroprotection by XCT 790 in an autophagy dependent mechanism, wherein (a) Representative western blot of LC3 processing assay in SHSY-5Y cells treated with XCT 790 (2 h) under growth condition and normalized LC3-II levels were quantified. β-tubulin was used as a loading control. (b) Representative microscopy images of tandem RFP-EGFP-LC3 assay in HeLa cells treated with XCT 790 for 2 h. Yellow puncta was autophagosomes and red was autolysosomes. Fold change in autophagosomes and autolysosomes by XCT 790 were quantified. Scale bar was 15 μm. (c) Graph indicating the cell viability read out of SHSY-5Y overexpressing EGFP-α-synuclein treated with XCT 790 in presence of pharmacological autophagy inhibitor 3-MA. Cell viability was analysed using CellTitre Glo (Promega) assay. More RLU readout was indicative of more cell viability and vice-versa. (d) Representative western blots of mTOR substrates like P70S6K (phospho and total form) and 4EBP1(phospho and total form) regulation by various treatments like XCT 790, EBBS and LiCl. β-tubulin was used as a loading control, (e) Representative western blots of signaling pathway proteins like AMPK (phospho and total form) and ULK1 (phospho and total form) regulation by XCT 790 and EBSS. β-tubulin was used as a loading control. Concentrations of XCT 790, 3-MA and LiCl used were 5 μM, 100 nm and 10 mM. Statistical analysis was performed using one-way ANOVA and post-hoc Bonferroni test. Error bars, mean±SEM. ns-non significant, **-P<0.01, ***-P<0.001.

In another embodiment of the present disclosure, FIG. 41 illustrates modulation of autophagy by XCT 790 through ERRα, wherein (a) ERRα, protein levels after transfecting either scrambled siRNA (100 picomoles) or ERRα siRNA (100 picomoles) for 48 h in HeLa cells was analyzed by western blotting and then quantified. β-tubulin was used as a loading control. (b and c) Microscopy images (b) of tandem RFP-EGFP-LC3 assay in XCT 790 treated HeLa cells (2 h) post ERRα siRNA transfection (48 h). Cells were immunostained for ERRα in various treatments. Scale bar was 15 μm. Quantification (c) of autophagosomes (Yellow puncta) and autolysosomes (red puncta) modulated by XCT 790 treatment in ERRα siRNA transfected cells. (d and e) Microscopy images (d) of tandem RFP-EGFP-LC3 assay in XCT 790 treated HeLa cells (2 h) post ERRα Flag transfection (48 h). Cells were immunostained for ERRα in all treatment groups. Scale bar used was 15 μm. Quantification (e) of autophagosomes (Yellow puncta) and autolysosomes (red puncta) modulated by XCT 790 treatment in ERRα Flag transfected cells. Concentration of XCT 790 used was 5 μM. Statistical analysis was performed using one-way ANOVA and post-hoc Bonferroni test. Error bars, mean±SEM. ns-non significant, **-P<0.01, P<0.001.

In another embodiment of the present disclosure, FIG. 42 illustrates localization of ERRα onto autophagosomes to modulate autophagy, wherein (a) Microscopy images of tandem RFP-EGFP-LC3 assay in HeLa cells transfected (48 h) with either ERRα siRNA or ERRα Flag treated with XCT 790 for 2 h. Cells were immunostained for ERRα. Scale bar was 15 μm. (b) PCC (Pearson's Colocalization Coefficient) analyses of ERRα with either autophagosome (yellow) or autolysosomes(red) in HeLa cells transfected (48h) with either ERRα siRNA or ERRα Flag treated with XCT 790 for 2 h were plotted. Statistical analysis was performed using one-way ANOVA and post-hoc Bonferroni test, Error bars, mean±SEM. ns-non significant, *-P<0.05, ***-P<0.001.

In another embodiment of the present disclosure, FIG. 43 illustrates neuroprotective effect of XCT 790 by degrading toxic protein aggregates through inducing autophagy in DAergic neurons of midbrain of mice, wherein (a) Representative photomicrographs of whole brain and SNpc for various cohorts namely vehicle, MPTP (23 mg/kg of body weight) and MPTP+Co (Co-administration of MPTP and XCT 790: MPTP; 2 mg/kg of body weight and XCT 790; 5 mg/kg of body weight). Scale bar is 600 μm. (b) Graph representing the unbiased stereological quantification of TH-ir DA neurons in SNpc for above mentioned cohorts. (c) Representative IHC photomicrographs of SNpc DAergic neurons double stained for A11 (toxic oligomer marker) and TH (SNpc marker) antibodies for the above mentioned cohorts. Scale bar is 50 μm. (d) Plot indicating the A11 puncta per DAergic neuron in SNpc was quantitated for all cohorts. (e) Representative fluorescent IHC photomicrographs of DAergic neurons in SNpc double stained for LC3 (autophagy 1.0 marker) and TH (SNpc marker) antibodies for various cohorts namely vehicle, MPTP, XCT only and MPTP+Co. Scale bar is 50 μm, (f) Graph representing the LC3 puncta per neuron for various cohorts.

In another embodiment of the present disclosure, FIG. 44 illustrates amelioration of MPTP induced behavioural impairments by XCT 790. Latency to fall for various cohorts such as vehicle, MPTP and MPTP+Co on both day 13 (a) and 15 (b) were monitored using rotarod test. (c) Representative trajectory maps were indicated for all the mentioned cohorts. (d and e) Plots indicating the peripheral distance travelled by mice were assessed through open field test on both day 13 (d) and 15 (e).

In another embodiment of the present disclosure, FIG. 45 illustrates non-toxicity of XCT 790 to yeast, wherein (a) Growth curve and its related parameters like growth rate (b) and doubling time (c) of XCT 790 treated WT EGFP. Growth rate and doubling time plots of XCT 790 treated WT α-syn-EGFP (d and e) and atg1Δ α-syn-EGFP (f and g) cells.

In another embodiment of the present disclosure, FIG. 46 illustrates modulation of starvation-induced autophagy by XCT 790 in yeast, wherein (a) Representative blot for GFP-Atg8 processing of XCT 790 treated yeast cells monitored across time points under starvation condition (2,4 and 6 h). Modulation of autophagy induction (total EGFP/PGK1) and autophagy flux (free EGFP/PGK2) upon XCT 790 treatment, were quantified and then plotted. PGK1 served as a loading control. (b) Scheme illustrating the protocol followed for α-synuclein degradation assay in yeast. Statistical analysis was performed using one-way ANOVA and post-hoc Bonferroni test. Error bars, mean±SEM. ns-non significant, *-P<0.05, ***-P<0.001.

In another embodiment of the present disclosure, FIG. 47 illustration non-toxicity of XCT 790 to cells (Hela and SHSY5Y) and scheme for α-synculein toxicity assay. Cell viability of cell lines like HeLa (a) and SH-SY5Y (b) after 72 h of XCT 790 treatments for various indicated concentrations. Cell viability was assayed using CellTitre Glo (Promega) kit.

In another embodiment of the present disclosure, FIG. 48 illustrates modulation of autophagy by XCT 790 in mTOR-independent manner in SH-SY5Y cells, wherein (a) Representative microscopy images of tandem RFP-EGFP-LC3 assay in SH-SY5Y cells treated with XCT 790 for 2 h. Yellow puncta was autophagosomes and red was autolysosomes. Fold change in autophagosomes and autolysosomes by XCT 790 were quantified and plotted. Scale bar was 15 μm. (b) Representative western blots of mTOR substrates like P70S6K (phospho and total form) and 4EBP1. (phospho and total form) regulation by various treatments like XCT 790, EBSS and LiCl in SH-SY5Y cells. β-tubulin was used as a loading control. Statistical analysis was performed using one-way ANOVA and post-hoc Bonferroni test. Error bars, mean±SEM. ns-non significant, *-P<0.05, **-P<0.01.

In another embodiment of the present disclosure, FIG. 49 illustrates that autophagic function of XCT 790 is unaffected in presence of actinomycin D, wherein (a) Representative microscopy images of tandem RFP-EGFP-LC3 assay in HeLa cells co-treated with XCT 790 and actinomycin D (act D). Scale bar 15 μm. (b) Fold change of autophagosomes and autolysosomes across various treatments were plotted. Statistical analysis was performed using one-way ANOVA and post-hoc Bonferroni test. Error bars, mean±SEM. ns-non significant, ***-P<0.001.

In another embodiment of the present disclosure, FIG. 50 illustrates administration of XCT 790 in mice MPTP toxicity model. (a) Dosage regimen of XCT 790 in various cohorts namely vehicle, MPTP (23 mg/kg of body weight) and MPTP Co (MPTP; 23 mg/kg of body weight and XCT 790; 5 mg/kg of body weight). (b) Plot indicating the densitometric quantification (B), measure of TH intensity in DAergic neurons. (c) Plot indicating the nigral volume was measured for the cohorts. Statistical analysis was performed using one-way ANOVA and post-hoc Bonferroni test, Error bars, mean±SEM. ***-P<0.001.

In another embodiment of the present disclosure, FIG. 51 illustrates scheme for the behavior study. Scheme indicating the dosage regimen of various cohorts such as vehicle (a), MPTP (b) and MPTP+Co (c) followed for the behavioral study.

### EXAMPLES

The present disclosure is further described with reference to the following examples, which are only illustrative in nature and should not be construed to limit the scope of the present disclosure in any manner.

Materials and Methods Employed in the Examples of the Present Disclosure

Chemicals and Antibodies

Yeast extract, peptone, dextrose, galactose and amino acids (leucine, lysine, methionine, histidine and uracil) are purchased from HiMedia.

3-MA (M9281), 6-Bio (B1686), LOPAC (LO1280), anti LC3 antibody (L7543), MPTP (methyl-4-phenyl-1, 2, 3, 6-tetrahydropyridine, M0896), DMEM (D5648), DMEM F-12 (D8900), Penicillin and Streptomycin (P4333), DAB (3, 3′-Diaminobenzidine, D3939), Atto 663 (41176) and Trypsin EDTA (59418C) are purchased from Sigma-Aldrich. Anti phospho P70S6K T389 antibody (9239) and total P70S6K antibody (9202), anti phospho GSK3β S9 antibody (5558) and total GSK3β antibody (9315), anti phospho4E-BP1T37/46 antibody (2855) and total 4E-BP1 antibody (9452), anti LAMP1 antibody (9091) and anti rabbitIgG, HRP (7074) antibody are purchased from Cell Signaling Technology. Anti β-Tubulin (MA5-16308) and anti GAPDH (MA5-15738) antibodies are purchased from Thermo Scientific.

Anti PGK1 (ab 38007) antibody is purchased from Abcam.

Anti GFP (11 814 460 001) antibody is purchased from Roche.

Anti Tyrosine Hydroxylase (N196) antibody is purchased from Santa Cruz Biotechnology.

Anti mouseIgG, HRP (172-1011) antibody is purchased from Bio-Rad. CMAC-Blue (C2110) is purchased from Life Technologies.

Bafilomycin A1. (11038) is purchased from Cayman chemical.

VECTASTAIN Elite ABC Kit (PK-6200) is purchased from VECTOR laboratories.

Plasmid Constructs and Yeast Strains

Plasmids used are pRS 316 GFP-Atg8, pRS 306 (α-synuclein-EGFP) under galactose promoter and pRS 306 (EGFP-βA₁₋₄₂), ptfLC3 (Addgene number. 21074), pRS 306 (EGFP-synuclein). Yeast strains employed in the instant disclosure are listed in Table 1 and the said yeast strains are obtained from EUROSCARF, Europe.

TABLE 1
List of yeast strains employed in the instant invention

| Strain | Genotype |
|---|---|
| sSNS 1 | BY4741; Mat α, his3Δ1 leu2Δ0, met15Δ0, ura3Δ0::EGFP |
| sSNS 14 | BY4741; Mat α, his3Δ1, leu2Δ0, met15Δ0, ura3Δ0::SNCA |
| | EGFP |
| sSNS 15 | BY4741 (atg1Δ); Mat α, his3Δ1, leu2Δ0, met15Δ0, |
| | ura3Δ0::SNCA EGFP |
| sSNS 50 | BY4741 (atg5Δ); Mat α, his3Δ1, leu2Δ0, met15Δ0, |
| | ura3Δ0::SNCA EGFP |
| sSNS 53 | BY4741 (atg8Δ); Mat α, his3Δ1, leu2Δ0, met15Δ0, |
| | ura3Δ0::SNCA EGFP |
| sSNS 54 | BY4741 (atg11Δ); Mat α, his3Δ1, leu2Δ0, met15Δ0, |
| | ura3Δ0::SNCA EGFP |
| sSNS 56 | BY4741 (atg15Δ); Mat α, his3Δ1, leu2Δ0, met15Δ0, |
| | ura3Δ0::SNCA EGFP |
| sSNS 57 | BY4741; Mat α, his3Δ1, let2Δ0, met15Δ0, ura3Δ0::GFP-Atg8 |

Culture and Culture Conditions
Yeast media used for culturing are YPD (Yeast extract, Peptone and Dextrose) for WT EGFP and autophagy mutant strains;
SD-Ura (Synthetic dextrose (2%) medium without uracil) for culturing α-synuclein-EGFP strain and GPP-Atg8 processing assay;
SG-Ura [Synthetic galactose (2%) medium without uracil] to induce α-synticlein-EGFP expression;
All the strains employed in the instant disclosure are cultured at 30° C. and 250 rpm.
HeLa cells are maintained in growth medium comprising of Dulbecco's Modified Eagle's medium (DMEM) (Sigma-Aldrich, D5648) supplemented with 3.7 g/L sodium bicarbonate plus 10% fetal bovine serum (FBS) (PAN, 3302-P121508) and 100 units/ml of penicillin and streptomycin (Sigma-Aldrich P4333) at 5% CO₂ and 37° C.
SH-SY5Y cells are maintained in DMEM-F12 containing 10% FBS (Life technologies). Cell lines are cultured in presence of 5% CO₂ and 37° C. To perform autophagy assays, equal numbers of sub-confluent HeLa cells are seeded in 6 well dishes, allowed to attach for 24 h, treated with 6-Bio (5 μM) and/or 3-MA (5 mM) in growth medium for 2 h.
For GFP-SNCA degradation assay, equal numbers of sub-confluent SH-SY5Y cells are seeded in 6 well dishes and allowed to attach for 24 h. Cells are transfected with GFP-SNCA plasmid using Lipofectamine 2000 (Life technologies) and allowed to express for24 h. Cells are treated with 6-Bio (5 μM) for 24 h and fold GFP-SNCA levels are analyzed using immuno blotting.
For tandem RFP-GFP-LC3B assay, sub-confluent cells are seeded in 60 mm dishes, transfected with ptf LC3B construct and allowed to express for 24 h. Later, cells are trypsinized, reseeded (105 cells) and allowed to attach on cover slips in a 12 well plate. Cells are treated with or without 6-Bio (5 μM) for 2 h and cover slips are processed for imaging.
Atg5+/+ and Atg5−/− MEFs34 are cultured in DMEM (ThermoFisher Scientific, 41965-039) supplemented with 10% FBS (ThermoFisher Scientific, 10270-106), 100 units/ml of penicillin and streptomycin (ThermoFisher Scientific, 15070-063) and 2 mM L-glutamine (ThermoFisher Scientific, 25030-024) at 37° C. humidified incubator under 5% CO₂.
S. cerevisiae transformation is done using lithium acetate method. Cells (˜108 cells) in early logarithmic phase of growth are harvested, resuspended in transformation mix (final concentrations: 33.3% PEG 3350, 0.1 M lithium acetate, 270 μg/ml salmon sperm DNA, 1-1.5 μg DNA) and subjected to heat shock at 42° C. for 40 minutes. Post heat shock cells are harvested and plated onto the selection media plates SD-URA for pRS306PPOT1-FLUC and SD-LEU for pRS305PPOT1-RLUC.
All procedures exemplified in the specification involving animal cell lines are approved by JNCASR institute Animal Ethical Committee (IAEC) and conducted as per guidelines of Committee for the Purpose of Control and Supervision of Experiments on Animals (CPCSEA). Inbred male C57BL/6 mice (3-4 months old) are used for all experimental groups.

Techniques Employed:

TCA Precipitation

All samples are collected in 12.5% TCA final concentration and stored at −80° C. for at least half an hour. Later, the samples are thawed on ice and centrifuged for 10 minutes at 16000 g and the pellet is washed with 250 μl of ice cold 80% acetone twice and air dried. This pellet is resuspended in 40 μl of 1% SDS-0.1N NaOH solution. Sample buffer (5×, 10 μl) is added to the lysate and boiled for 10 minutes before loading.

Immunoblotting

Total cell lysates are electrophoresed on different 1 percentages of SDS-PAGE based on the desired protein size and transferred onto PVDF membrane at constant current of 2 Ampere for 30 minutes (Transblot turbo, Bio-Rad Inc, USA). Transfer is confirmed by Ponceau S staining of blot and the blots scanned are used as loading controls. Blots are incubated overnight with 5% skim milk in primary antibody (Anti-GFP, Roche #11814460001, Anti-MAP1LC3B, CST #L7543). Secondary antibody used at 1:10.000 is goat anti-mouse (Bio-7 Rad #172-1011) or goat anti-rabbit antibody (Bio-Rad #172-1019) conjugated to HRP. Blots are developed by using ECL substrate (Thermo Scientific #34087 or Bio-Rad #170-5061) and images captured using auto capture program in Syngene G-Box, UK. image J (NIH) are used for quantitation of band intensities.

For mammalian cells, following appropriate treatments, cells are washed with ice cold PBS. Cells are then lysed in 100 μl of sample buffer (10% w/v SDS, 10 mM DTT, 20% v/v glycerol, 0.2 Tris-HCL pH 6.8, 0.05% w/v bromophenol blue) and then collected using a rubber cell scraper. The lysates are boiled at 99° C. for 15 minutes and stored at −20° C.

Western Blotting

Western blotting is performed using standard methods. Immunoblotting in MEFs is carried out as described previously. Dilutions of primary antibodies used are as follows: Anti-p62 1:1000 (Progen Biotechnik, GP62-C), Anti-MAP1LC3B 1:3000 (Nevus Biologicals, NB100-2220) and Anti-GAPDH (Cell Signalling Technologies, 2118S). Secondary antibodies conjugated to HRP are used at 1:10000 dilution as follows: Anti-Guinea-Pig-HRP (Abeam, ab50210) and Anti-Rabbit-HRP (Calbiochem, 401393).

Pexophagy Assay

Pot1-GFP positive strains are allowed to grow till the A600 reached 0.8-1 in YPD. Peroxisome biogenesis is induced by growing these cells in oleate medium (0.1% oleate, 0.5% Tween-40, 0.25% yeast extract, 0.5% peptone, and 5 mM phosphate buffer) for 12hours. Cells are harvested, washed twice to remove traces of oleate and transferred to starvation medium without nitrogen, at inoculum density A600 3, to induce pexophagy. Cells are collected at various time intervals after pexophagy induction and processed by TCA method.

Immunofluorescence

Appropriate number of cells are plated on top of coverslips placed in 65 mm cell culture dishes for transfection. Transfected cells are divided into different treatment groups. Post treatment, cells are washed with PBS and fixed in 4% paraformaldehyde and permeabilized using 0.25% Triton X-100, Overnight incubation with Anti-p62/SQSTM1 (rabbit polyclonal, #PM045), Anti-EEA1 (rabbit polyclonal, CST #3288) is done at 4° C. Excess antibody is washed with PBS and coverslips are incubated with Atto-633 (goat anti-rabbit IgG, Sigma #41176). The coverslips are mounted with VECTASHIELD antifade reagent (H-1000/H-1200, Vector laboratories). Imaging for HeLa cells is carried out using Delta vision microscope (Olympus 60×/1.42, Plan ApoN, excitation and emission filter Cy5, FITC and TRITC, polychroic Quad).

Immunofluorescence analysis in MEFs is carried out by fixing the cells with 4% methanol free paraformaldehyde for 15 minutes, permeabilised with 0.5% TritonX-100 in PBS for 10 minutes, and then blocking with 5% FES in PBS for 30 minutes at room temperature, along with PBS washes in between every steps. Anti-p62 antibody (Progen Biotechnik, GP62-C) is used at 1:250 dilution in 5% PBS in PBS and incubated overnight at 4° C. Cells are then washed and incubated with goat anti-guinea pig Alexa 594 (ThermoFisher Scientific, A-15 11076) secondary antibody at 1:1000 dilution for 1 hour at room temperature. Cells are washed, counterstained with DAPI (ThermoFisher Scientific, D1306) in PBS for 5 minutes, washed again and then mounted using Prolong diamond anti-fade reagent (ThermoFisher Scientific, P36970). Slides are imaged using a Zeiss LSM 510 Meta Confocal Microscope using 100× objective. Analysis is performed by assessing for the percentage of cells displaying an accumulation of endogenous p62-positive aggregates.

Analysis of Autophagosome Maturation Using mRFP-GFP-MAP1LC3B Reporter

Transfection is done on a 60 mm dish with HeLa cells at 60-70% confluency. Cells were transfected with tandem RFP-GFP-MAP1LC3B construct (Addgene plasmid #21074) using 5 μl of Lipofectamine 2000 (11668-019, Invitrogen) and 2.5 μg of DNA (2:1 ratio) diluted in 100 μl of OPTI-MEM (31985-070, Invitrogen) separately. 72 hours after transfection cells are either left untreated or treatment with various concentrations of Bay11-7082 ZPCK is done for 2 hours. Starvation is induced by treating cells with Earle's balanced salt solution (EBBS). After treatment, cells are fixed in 4% paraformaldehyde and permeabilized using 0.25% Triton X-100, The coverslip is mounted with VECTASHIELD antifade reagent (H-1000, Vector laboratories). Imaging for HeLa cells is carried out using Delta vision microscope (Olympus 60×/1.4, Plan ApoN, excitation and emission filter Cy5, FITC and TRITC, polychroic Quad).

EGFR Trafficking

HeLa cells are plated on 6 well plates and allowed to attach on the surface. The cells are washed with PBS and then starved in DMEM (serum free media) for 3 hours. Pre-treatment with compounds is carried out for 1 hour, following which they are pulsed with 100 ng/ml of EGF and samples are collected at 0, 1, 2 and 3 hours.

Quantification of Cells with Increased p62+ Aggregates

Analysis of p62 aggregates is done as described previously. Briefly, immunofluorescence analysis with anti-p62 antibody is performed for assessing endogenous p62+ aggregates using confocal microscope. The percentage of cells with increased p62+ aggregates is quantified by assessing 200 cells per condition from independent experiments, in which a cell with an accumulation of p62+ aggregates was given a score of 1 whereas a cell having basal (low) levels of p62+ aggregates was given a score of 0.

Mean Intensity Calculation

ImageJ software (NIH) is used to calculate the mean intensity. Images are opened using the split channel plugin. Co-localization plugin in the analysis tools is used to obtain the colocalized area between two channels as a separate window. The intensity is calculated using the measure plugin in analysis tools.

Statistical Analysis Employed in the Instant Disclosure

Statistical analyses are performed using unpaired Student's t-test and ANOVA (one-way or two-way or both) followed by post-hoc Bonferroni test in GraphPad Prism. Error bars are expressed as mean±SEM.

Image Preparation

Yeast and mammalian images were prepared using Softworx software (GE healthare). Lace plant MIP images were prepared using NIS elements software (Nikon, Canada). Images were plated using Adobe Photoshop CC. Fluorescent MIP images had their brightness and contrast modified equally using Adobe Photoshop CC.

### Example 1

### Assay for Monitoring Autophagy in Real Time and use of the Assay for Screening the Small Molecules

The S. cerevisiae shuttle vectors pRS306 (URA) and pRS305 (LEU) are used to clone the POT1 promoter and the Firefly and Renilla luciferase genes, respectively. The oleate responsive region of the POT1 promoter is amplified from yeast genomic DNA and along with the firefly and Renilla luciferase genes (firefly gene from pMY30 and Renilla gene from pRL-TK) is cloned into these vectors to obtain the constructs pPM3 and pPM5. These plasmid constructs are linearized using suitable restriction enzymes in the selection marker and transformed into wild type strains of S. cerevisiae and P. pastoris by standard transformation methods-Lithium acetate or PEG based transformation.

The transformed colonies of S. cerevisiae and P. pastoris are then tested for firefly luciferase activities. The colonies positive for firefly are then co-transformed with Renilla luciferase vector and tested for its activity,

The vectors are transformed into S. cerevisiae and P. Pastoris haploid strains including wild-type (WT), and the atg1. (systematic gene name, YGL180W), atg5 (YPL149W) and atg36 deletion strains (Gietz and Woods 2002). The wild type and the deletion mutants are from the MATa collection, created by the. Saccharomyces Genome Deletion Project. These strains are blocked in all autophagy related-pathways, including pexophagy.

To validate the luciferase assay developed in the laboratory, it is compared to the conventional Pot1-GFP processing assay, Both pexophagy and general autophagy are measured by following the rates of degradation of firefly and Renilla activities (FIG. 1A).

In the luciferase assay, the activity for the wild type cells goes down with time whereas the mutant shows no decrease in the activity which resembles the western for Pot1-GFP processing assay (FIG. 1B).

Z-factor is calculated for 5 assays done in triplicates in 384 well format for both firefly as well as Renilla luciferase activities. Z-factor for Fluc=0.8628±0.03481 and Z-factor for Rluc=0.8224±0.03879, which suggests a very good assay which when scaled up to millions of compounds would give very less false positives and better reliability.

The wild type cells show a gradual decrease in luciferase counts upon induction of autophagy whereas core autophagy mutants atg1 and atg5 and selective autophagy mutant atg36 (adaptor protein for pexophagy) do not show any drop in the luciferase activity over time (FIG. 1B).

Screening of Small Molecule Libraries Identified Several Putative Modulators of Autophagy

After validation of the luciferase assay, two small molecule libraries are screened for their effect on autophagy. The library from Sigma contains 1280 FDA approved drugs and Enzo library of natural compounds has 502 small molecules.

To screen small molecule library LOPAC1280 in S. cerevisiae SNCA toxicity model, working plates containing 50 μM drugs in 1.5% DMSO (190 compounds/plate) are prepared in a 384 well format. WT SNCA-GFP with or without small molecules and untreated WT GFP are grown under optimized conditions (80 μl, 30° C. and 420 rpm) for 36 h in a plate reader (Varioskan Flash, Thermo Scientific) in duplicates with automatic absorbance (A600) recording every 20 min. Growth curves of untreated WT GFP and WT SNCA-GFP strains are plotted and mid to late exponential phase time point of untreated WT GFP strain is chosen as reference for data analysis. Its corresponding time point of untreated and drug treated WT SNCA-GFP strains are plotted separately in a box plot. Small molecules that rescued the growth lag by ≥3 SD units of untreated WT SNCA-GFP strain are considered as ‘Hits’.

Thus, a 3 standard deviation (SD) parameter is used as a criterion to obtain the hits from the primary screen. Primary screening also identifies several known autophagy modulators as hits (FIG. 2A). Overall, 10 putative inhibitors and 7 autophagy enhancers are obtained from the primary screening. Two of the inhibitors and two of the activators are further validated using secondary assays for their role in autophagy. Both the inhibitors show a dose dependent inhibition in autophagy as determined using luciferase assay wherein inhibition by concentrations 1 μM, 10 μM, 25 μM and 50 μM of the inhibitors are assessed and inhibition is observed to increase as concentration increases from 1 μM to 50 μM. (FIG. 2B).

Small Molecule Screening Reveals 6-Bio as a Potent Inducer of Autophagy

In Yeast Cells:

The occurrence of protein aggregates and cytotoxicity by SNCA overexpression is recapitulated in the budding yeast, Saccharomyces cerevisiae.

The yeast model is employed to screen for small molecules that prevent cytotoxicity by aggregate degradation (FIG. 1A). Of the hits that rescue growth in this model is Agk2 which is shown to rescue SNCA toxicity affirming the reliability of the assay and the 6-Bio [(2′Z,3′E) -Bromoindirubin-3′-oxime] (FIG. S3A). 6-Bio does not affect the growth of yeast cells (FIG. 1B). In order to understand the involvement of 6-Bio in autophagy, GFP Atg8 (GFP tagged Autophagy related protein 8, yeast autophagosome marker) processing assay under both growth and starvation conditions are employed. During growth conditions where autophagy is barely detectable, 6-Bio dramatically induces autophagy (6 h time point, P<0.001 versus untreated; FIG. 1C) and also the flux (6 h time point, P<0.001 versus untreated; FIG. 1C). Similarly, 6-Bio treatment under starvation condition shows significant increase in autophagy induction (4 h and 6 h time points, P<0.001 versus untreated; FIG. 1D) and flux (4 h and 6 h time points, P<0.01 and P<0.001 respectively versus untreated; FIG. 1D) by 2-fold in a time dependent manner suggesting 6-Bio augmented starvation induces autophagy.

In Mammalian Cells:

MAP1LC3B/LC3B (Microtubule-associated protein 1A/1B light chain 3B, a mammalian autophagosome marker) processing and tandem RFP-GFP-LC3B assays are employed. In HeLa cells, 6-Bio increases LC3B-II (processed form of LC3B-I) levels in a dose dependent manner suggesting autophagy modulation (FIG. 3A). In the presence of lysosomal protease inhibitors, E64D and Pepstatin A, LC3B-II accumulated is significantly more than that of 6-Bio only and/or E64D and Pepstatin A only validating that 6-Bio is indeed an autophagy enhancer. In the tandem RFP-GFP-LC3B assay that reveals autophagy flux, 6-Bio treatment dramatically increases autolysosome numbers (˜9 fold, P<0.001, compared to control; FIG. 1E) indicating enhanced fusion of autophagosomes with lysosomes.

Effect of 6-Bio on Lysosomal Function

HeLa cells are treated with 6-Bio and/or E64D and Pepstatin A for 2 h, followed by treatment with lysotracker for 20 min. Lysotracker fluorescence intensity is reduced in presence of protease inhibitor like E64D and Pepstatin A. The fluorescence intensity of lysotracker is found to be comparable between untreated and 6-Bio treated cells. Thus, no difference in both E64D +Pep A only and 6-Bio+E64D and Pep A treatments is found. Lysotracker staining indicates that there was no change in lysosome acidification. LAMP1 (Lysosomal-associated membrane protein 1) positive vesicle intensities and distribution also are unaltered upon 6-Bio treatment suggesting that perhaps lysosomal functions are not perturbed by 6-Bio.

To address if the molecule enters into cell via endocytosis, we carried out the tandem RFP-GFP LC3B assay at 16° C. At this temperature, endocytosis pathway is highly reduced as evident by the significantly decreased cellular uptake of FITC-Dextran (70 kDa) as compared at 37° C. However, the effect of 6-Bio in increasing fusion between autophagosomes and lysosomes at 37° C. (˜10 fold, control vs 6-Bio treated, P<0.001, FIGS. 11, A and B) and 16° C. (˜8 fold, control vs 6-Bio treated, P<0.001) are comparable suggesting that endocytosis does not play a predominant role in the action of 6-Bio in modulating autophagy.

These results suggest that 6-Bio affects autophagy independent of endocytosis perhaps by passive diffusion. From these two model systems, we noticed that 6-Bio not only induces autophagy but also enhances starvation induced autophagy and strikingly promotes autolysosome formation without perturbing the lysosomal function.

### Example 2

### Effect of 6-Bio on Yeast

Growth assays: In a 384 well plate, appropriate yeast strains are seeded (A₆₀₀˜0.07) with and without drugs and incubated (80 μl, 30° C. and 420 rpm) in a multiplate reader (Varioskan Flash, Thermo Scientific) for 48 h with automatic absorbance (A₆₀₀) recording every 20 min. Growth curves are plotted and analyzed using GraphPad Prism.

Induction of α-synuclein-EGFP aggregates: To induce α-synuclein-EGFP aggregates, the corresponding strains are inoculated in SD-Ura medium, Secondary culture is inoculated from primary inoculum and incubated till A₆₀₀ reaches 0.8/ml. Cells are washed twice with sterile water and aggregates are induced by adding SG-Ura for 12-16 h.

To screen small molecule library LOPAC¹²⁸⁰ in S. cerevisiae α-synuclein toxicity model, working plates containing 50 μM drugs (small molecule) in 1.5% DMSO (190 compounds/plate) are prepared in a 384 well format. WT α-synuclein-EGFP with and without small molecules and untreated WT EGFP are grown under optimized conditions (80 μl, 30° C. and 420 rpm) for 36 h in a plate reader (Varioskan Flash, Thermo Scientific) in duplicates with automatic absorbance (A₆₀₀) recording every 20 min.

Growth curves of untreated WT EGFP and WI α-synuclein-EGFP strains are plotted and mid to late exponential phase time point of untreated WT EGFP strain is chosen as reference for data analysis. Its corresponding time point of untreated and drug treated WT α-synuclein-EGFP strains are plotted separately in a box plot. Small molecules that rescue growth lag due to α-synuclein toxicity by ≥3 SD units of untreated WT α-synuclein-EGFP strain are considered as ‘Hits’.

Of the hits that rescue growth lag due to α-synuclein toxicity in this model are Agk2 which is known to rescue growth lag due to α-synuclein toxicity (T. F. Outeiro et al.) affirming the reliability of the assay and the 6-Bio [(2′Z,3′E)-6-Bromoindirubin-3′-oxime](P. Polychronopoulos et al.) (FIG. 8A). Interestingly, 6-Bio does not affect the growth of yeast cells (FIG. 3B). It is observed that 6-Bio rescues the growth lag due to u-synuclein toxicity in a more statistically significant manner than AGK2. and 6-Bio has increased efficiency for inducing autophagy and massively enhancing starvation induced autophagy.

In order to understand the involvement of 6-Bio in autophagy, GFP-Atg8 (an autophagosome marker) processing assay under both growth and starvation conditions is employed.

GFP-ATG8 Processing Assay Protocol:

Saccharomyces cerevisiae strain containing the GFP-Atg8 (pRS 316 vector backbone) plasmid is grown in synthetic complete medium lacking uracil (SC-URA) under appropriate conditions (30° C. 250 rpm). From this, a secondary culture is inoculated at A600 nm=0.2 and grown as above until A600 nm reached ˜0.65. The cultures are transferred to SD-N (nitrogen starvation) medium at A600 nm=3, separately with and without the compounds, and samples are collected at different time intervals. Sample preparation is done by the TCA precipitation method and immunoblotting is performed using standard methods.

Preparation of Yeast Lysates for Immunoblot Analysis:

Yeast strains (A₆₀₀=3) are resuspended in trichloroacetic acid (12.5%) and stored at −80° C. for at least half an hour. Samples are thawed on ice, centrifuged (16000×g, 10 min) and pellet is washed twice with ice-cold acetone (80%). Pellets are air dried, resuspended in lysis (1% SDS and 0.1 N NaOH) solution and Laemmii buffer and boiled for 10 min.

To assess α-synuclein-EGFP degradation efficacy by 6-Bio, after inducing α-synuclein-EGFP aggregate, galactose promoter is turned-off by adding dextrose. Cells are treated with 6-Bio (50 μM) for 0, 6, 12 and 24 h. Subsequent degradation of α-synuclein-EGFP levels are analyzed using immunoblotting (FIG. 9A).

Yeast cultures after respective treatments are washed, mounted on agarose (2%) pad and imaged. Images are acquired using DeltaVision Elite widefield microscope (API, GE) with following filters: DAPI (390/18 and 435/48), FITC (490/20 and 529/38), TRITC (542/27 and 594/45) and Cy5 (632/22 and 676/34). Images are processed using DV SoftWoRX software. Autophagosome (yellow) and autolysosome (red) are counted using Cell Counter plug-in in imager software (NIH) and graphically represented as fold difference versus untreated (FIGS. 4A, B, C and D).

Assays carried out to monitor degradation of α-synuclein-EGFP aggregates in presence of 6-Bio under non-starvation and starvation conditions revealed a time-dependent and significant decrease in the α-synuclein-EGFP levels in wild type cells (FIGS. 4B and D and FIGS. 9 B and C) but not in an autophagy mutant, atg1Δ (FIGS. 3C and E and FIGS. 9D and E). These results suggest that 6-Bio treatment is not only able to enhance starvation induced autophagy but also resulted in a concomitant decrease of α-synuclein-EGFP demonstrating that the pro-survival effects of 6-Bio is due tip autophagy dependent α-synuclein-EGFP clearance.

During growth conditions where autophagy is barely detectable, 6-Bio dramatically induces autophagy (6 h time point, P<0.001 versus untreated; FIG. 3C) and also increases the flux (6 h time point, P<0.001 versus untreated; FIG. 3C) by approximately 3 fold across all time points. Similarly, 6-Bio treatment under starvation condition shows significant increase in autophagy induction (4 h and 6 h time points, P<0.001 versus untreated; FIG. 3D) and flux (4 h and 6 h time points, P<0.01 and P<0.001 respectively versus untreated; FIG. 3D) by 2-fold in a time dependent manner suggesting 6-Bio augmented starvation induced autophagy.

### Example 3

### Assessing the Effect of 6-Bio in Autophagy in Mammalian Cells

HeLa cells are maintained in DMEM containing 10% FBS (Pan-Biotech), SH-SY5Y cells are maintained in DMEM-F12 containing 10% FBS (Life technologies). Cell lines are cultured in presence of 5% CO₂ and 37° C.

To perform autophagy assays, equal numbers of sub-confluent HeLa cells are seeded in 6 well dishes, allowed to attach for 24 h, treated with 6-Bio μM) and/or 3-MA (5 mM) in growth medium for 2 h. For EGFP-α-synuclein degradation assay, equal numbers of sub-confluent SH-SY5Y cells are seeded in 6 well dishes and allowed to attach for 24 h. Cells are transfected with EGFP-α-synuclein plasmid using Lipofectamine 2000 (Life technologies) and allowed to express for 24 h. Cells are treated with 6-Bio (5 μM) for 24 h and fold EGFP-α-synuclein levels are analyzed using immunoblotting.

Tandem RFP-EGFP-LC3 Assay

For tandem RFP-EGFP-LC3 assay, sub-confluent cells are seeded in 60 mm dishes, transfected with ptf LC3 construct and allowed to express for 24 h. Later, cells are trypsinized, reseeded (10⁵ cells) and allowed to attach on cover slips in a 12 well plate. Cells are treated with and without 6-Bio (5 μM) for 2 h and cover slips are processed for imaging.

It is observed that in HeLa cells, 6-Bio increases LC3-II (processed form of LC3-I) levels in a dose dependent manner suggesting autophagy activation (FIG. 5A). Through the tandem RFP-EGFP-LC3 assay that reveals autophagy flux, it is observed that 6-Biotreatment dramatically increases autolysosome numbers (˜9 fold, P<0.001, compared to control; FIG. 3E) indicating enhanced fusion of autophagosomes with lysosomes.

Preparation of Mammalian Cell Lysates for Immunoblot Analysis

After treatments, cells are collected in Laemmli buffer to perform LC3 processing assay, P70S6K, GSK3β and 4E-BP1 immunoblotting. To validate EGFP-α-synuclein degradation by 6-Bio, treated cells are scraped and collected in growth medium. After washing with phosphate buffer, pellets are lysed in Laemmli buffer and boiled for 10 min.

Samples are electrophoresed on SDS-PAGE (8-15%) and then transferred onto PVDF (Bio-Rad) membrane using Transblot turbo (Bio-Rad). Transferred blots are stained with Ponceau S, probed with appropriate primary antibodies overnight and subsequently horseradish peroxidase conjugated secondary antibody. Signals are developed using enhanced chemiluminescence substrate (Clarity, Bio-Rad), imaged using a gel documentation system (G-Box, Syngene) and bands are quantified using image software (NIH). (FIGS. 4F and 5A)

It is observed that 6-Bio significantly reduces EGFP-α-synuclein levels (˜2 fold, P<0.001versus untreated).

Effect of 3-MA on EGFP-α-Synuclein Clearance by 6-Bio

In the presence of autophagy inhibitor 3-methyl adenine (3-MA) (5 mM for 2 hours), the EGFP-α-synuclein levels does not change upon 6-Bio co-treatment suggesting that autophagy is the primary mechanism for degradation (FIGS. 4C and E; FIG. 4F and FIGS. 9D and E). Similar effect is demonstrated in yeast model in the instant disclosure.

Effect of 6-Bio on mTOR Signalling

As mTOR negatively controls autophagy, it is tested if 6-Bio affected mTOR signaling. 6-Bio decreases phosphorylation levels of P70S6 kinase and 4E-BPI in a dose dependant manner (FIG. 5A), indicating 6-Bio negatively regulates mTOR signaling. These assays confirmed that 6-Bio treatment not only induces autophagy but also enhances the autophagic flux by promoting autophagosome fusion with lysosomes in an mTOR dependent manner.

Microscopy

For mammalian cell microscopy, after 2 h of treatment, coverslips are fixed using 4% paraformaldehyde (PFA) (Sigma) and permeabilized using Triton X-100 (0.2%, HiMedia). Coverslips are mounted using antifade, Vectashield (Vector laboratories). For antibody staining, coverslips are blocked in 5% BSA for 1 h, incubated in primary antibody overnight and subsequent probing with fluorescent conjugated antibody. Coverslips are mounted using antifade, Vectashield (Vector laboratories).

Images are acquired using DeltaVisionElitewidefield microscope (API, GE) with following filters: DAPI (390/18 and 435/48), FITC (490/20 and 529/38), TRITC (542/27 and 594/45) and Cy5 (632/22 and 676/34). Images are processed using DV SoftWoRX software. Autophagosome (yellow) and autolysosome (red) are counted using Cell Counter plug-in in ImageJ software (NIH) and graphically represented as fold difference versus untreated (FIG. 3E).

Cell Viability Assay

SH-SY5Y cells are seeded on a 96 well plate and transfected with GFP-SNCA only and/or is cotransfected with shRNA GSK3B and GFP-SNCA. To cells, the drugs were added (24 h) after 48 h of transfection. Then, the cell viability was measured using the CellTitre-Glo® (Promega) and luminescence was measured using Varioskan Flash (Thermo Scientific).

Autophagy Dependent GSK3B Mediated Neuro(Cyto) Protection by 6-Bio

Significant reduction of GSK3B activity upon 6-Bio treatment is observed as revealed by the reduced p-GSK3B levels (FIG. 2F and FIG. 3A). To understand the GSK3B dependency of autophagic activity by 6-Bio, knockdown studies in SH-SY5Y cells are performed. For this, shRNA GSK3B is transiently transfected to cells and after 48 h, the GSK3B protein levels are reduced significantly. In SH-SY5Y cells, when SNCA is over expressed for 72 h, the cell viability is significantly affected. When these cells are treated with 6-Bio, its viability is significantly increased and comparable to that of control. Similar protection is noted in GSK3B silenced SNCA over expressed cells. When GSK3B expression is silenced, addition of 6-Bio only shows marginal cytoprotection as compared to untreated cells with normal GSK3B expression. In fact, in silenced cells, 6-Bio is not effective in cytoprotection over and above that offered by silencing GSK3B but is cytotoxic. In addition, another GSK3B inhibitor, compound VIII also exerts protection against SNCA mediated toxicity. Using a direct readout for autophagy (tandem RFP-GFP-LC3B assay), a similar silencing strategy is employed to address the GSK3B and autophagy interplay in presence of 6-Bio. In GSK3B silenced cells, the autolysosomes are increased with concomitant reduction in autophagosomes than that of its scrambled shRNA control (P<0.001). Interestingly, the autophagosomes and autolysosomes formed in GSK3B silenced cells are similar to that of 6-Bio treatment. Notably, the autolysosomes formed in 6 Bio treated GSK3B silenced cells are significantly reduced than that of only treated cells (P<0.001). Although 6-Bio is known to affect other signaling pathways such as PDK1 (Pyruvate dehydrogenase lipoamide kinase isozyme 1) and JAK/STAT3 (Janus kinase/signal transducers and activators of transcription 3) results of the study suggest that 6-Bio primarily modulates autophagy in a GSK3B dependent manner.

### Example 4

### Studies on MPTP Mouse PD Model for 6-Bio

Mice are randomly allocated to 5 different study groups, viz., Control (n=5), 6-Bio-only (n=5), MPTP only (n=5), MPTP+Prophylactic (2 days prior) administration of 6-Bio (n=3) and MPTP+Co-administration of 6-Bio (n=5).

As described by Vernice Jackson-Lewis & Serge Przedborski (2007), 23.4 mg/kg MPTP.HCl (equivalent to 20 mg/kg free base) in 10 ml/kg body wt. of saline is administered intraperitoneally (i.p.) for 4 times at 2 h intervals.

Further, 5 mg/kg body wt. of 6-Bio in 100 μl of saline is administered i.p. to the MPTP-injected animals by following either of the two different regimen; first regimen comprised of a prophylactic/pre-treatment which is begun two days prior to MPTP administration (MPTP+Pro); while the second involved treatment given alongside the MPTP injection (MPTP+Co). In both the cases, 6-Bio is administered for 7 days post-MPTP administration daily (FIG. 10A). The other experimental groups include 6-Bio only (daily i.p.) and MPTP alone (daily vehicle) for 9 days. All mice are sacrificed 9 days post MPTP administration and brains are processed for immunohistochemistry.

Tyrosine Hydroxylase (TH) Immunohistochemistry:

Mice are anaesthetized using Halothane BP (Piramal Healthcare) inhalation and perfused intracardially with normal saline followed by 4% PFA in 0.1 M phosphate buffer, pH 7.4. Brains are removed quickly and post-fixed with 4% PFA for 24 h-48 h at 4° C. Following cryoprotection in 15% and 30% sucrose, 40 μm thick coronal cryosections of midbrain are collected serially on gelatinized slides. Immunoperoxidase labelling protocol as identical to that reported. Briefly, endogenous peroxidase is quenched using H₂O₂ (0.1%) in methanol (70%), followed by blocking of non-specific staining by buffered solution (3%) of bovine serum albumin for 4 h at room temperature. Sections are incubated with TH primary antibody (1:500) followed by biotin conjugated secondary antibody (1:200 dilution, Vector Laboratories). Tertiary labelling is performed with avidin-biotin complex solution (1:100, Vector Laboratories). Staining is visualized using 3′-3′-diaminobenzidine (0.05%) as a chromogen in a solution of 0.1M acetate imidazole buffer (pH 7.4) and H₂O₂ (0.1%). Negative controls are processed identically, except that primary antibody is replaced with dilution buffer.

Stereological Quantification of TH⁺ Dopaminergic Neurons in SNpc:

As described by Y. Fu et al., SNpcis delineated using the 4× objective of Olympus BX61 Microscope (Olympus) equipped with Stereo Investigator Software Version 7.2 (MicroBrightField). Stereological quantification is performed using optical fractionator probe with slight modifications(P. A. Alladi et al.). Briefly, every sixth midbrain section containing SNpc is chosen and TH⁺ cells are counted under 100× objective, with a regular grid interval of 22500 μm² (x=150 μm, y=150 μm) and counting frame with an area of 3600 μm² (x=60 μm, y=60 μm) (FIG. 10B). Mounted thickness is determined at every fifth site, averaging to 25 μm. Guard zone 4 μm is applied on either side, thus providing 17 μm of z-dimension within optical dissector. The quantification is performed starting with first anterior appearance of TH⁺ neurons in SNpc to caudal most part. The SNpc volume is determined by planimetry (FIG. 10C) and total numbers of neurons according to mean measured thickness are noted.

Densitometry Based Image Analysis:

High magnification images of TH stained nigral DAergic neurons, captured for offline assessment of Tyrosine Hydroxylase (TH) enzyme expression levels=used. Expression intensity is measured using a Windows based image analysis system (Q Win V3, Leica Systems). Cumulative mean is derived from the values obtained from sampling, approximately 200 DAergic neurons per animal. Intensity output is measured on a grey scale of 0-255, where 0 equals intense staining and 255 means absence of staining. Thus, lower grey values suggest higher protein expression and vice-versa.

From the above assays, it is observed that number and health of DA neurons (as revealed by tyrosine hydroxylase (TH) staining intensities) are significantly reduced in MPTP treated mice (˜3 fold, P<0.001 compared to control; FIG. 5B to D and FIG. 10B). Strikingly, in both the 6-Bio treatment regimen, number of DA neurons resembles almost that of control or animals injected with 6-Bio only (Co/Pro ˜2.5 fold versus MPTP, P<0.001; FIG. 5B to D and FIG. 10B). In addition, the decrease in SNpc volume upon MPTP administration is not seen in 6-Bio treated groups (P<0.001 versus Co, P<0.01 versus Pro; FIG. 10C). These observations in mouse model of PD reassert the neuroprotective nature of 6-Bio and activation of autophagy.

In Vivo Blood Brain Barrier Assay:

Animals are randomly allocated for placebo control and drug treatment cohorts, C57BL/6 mice are injected with placebo control or 6-Bio (5 mg/kg of body weight, intraperitoneally) twice with 24 h time interval. After second injection, the brains are harvested at 15 min, 30 min, 60 min, 6 h, 12 h and 24 h by cervical dislocation.

Mice brains are immediately homogenized with RIPA buffer (with protease inhibitor cocktail, Roche) for both treatment cohorts, Homogenously macerated mouse brain sample (100 μl) is mixed with acetonitrile (ACN, 400 μl), formic acid (0.2%) and aceclofenac (100 ng/ml, an internal standard), vortexed for 10 min at 2000 rpm Orbital shaker. Then, samples are centrifuged and supernatant is injected to LCMS/MS.

Standard samples are prepared by spiking 6-Bio standard in to blank control brain sample.

Standard spiking is performed such that resultant concentrations are 0, 10, 100, 500, 1000, 1500 and 2000 ng/ml of drug in blank brain matrix. For LC, PE 200 (Perkin Elmer) HPLC with Agilent Zorbax XDB C8 4,6×75 mm, 3.5 μm column is employed. The following conditions are used for LC: Mobile phases (0.1% formic acid in water (5%): Methanol (95%), Isocratic flow rate (0.7 ml/min), Run time (4 min), Injection volume (15 μl) and Needle wash solution (1:1 methanol:water mix containing 0.1% thrmic acid). The mass spectrometry (API3000, AB Sciex) is used with aceclofenac as an internal control and data were processed using Analyst Software V1.4.2, Drug injections of the various cohorts and preparations of the brain homogenates are performed at JNCASR. ACQUITY LABORATORIES performed the LC MS/MS analysis of the brain samples.

6-Bio Enhances Autophagy and Clears Toxic Protein Aggregates in Mice Brain:

The autophagy and toxic aggregate levels in the DAergic neurons in SNpc of midbrain are evaluated. A significant reduction of LC3B puncta per neuron in MPTP treated cohort than that of placebo (˜1.8 fold, placebo vs MPTP, P<0.001, FIGS. 4A and B) is observed. On the other hand, A11 puncta are significantly higher (˜7 fold, placebo vs MPTP, P<0.001, FIGS. 4, C290 and D). Along with decreased autophagy, these SNpc (TH+) neurons display toxic aggregate build-up. This reduced autophagy upon administration of MPTP is in agreement with previous observations. Furthermore, as seen in cell culture models, 6-Bio treatment alone increases LC3B puncta per neuron (˜2 fold, placebo vs 6-Bio only, P<0.001, FIGS. 4A and B) suggesting that 6-Bio could induce autophagy in mice brain by crossing the blood-brain barrier.

When 6-Bio is co administered along with MPTP, LC3B puncta per neuron increases (˜2,5 fold, MPTP vs MPTP+Co, P<0.001, FIGS. 4A and B) with a significant reduction in all positive aggregates (˜7 fold, MPTP MPTP+Co cohort, FIGS. 4C and D). Strikingly, the aggregate numbers in MPTP+Co are found to be comparable to that of control suggesting that the 6-Bio treatment decreases the toxic aggregates to that of placebo neurons (placebo MPTP±Co, ns, P>0.05, FIGS. 4C and D), These results imply that autophagy is drastically induced in the MPTP and 6-Bio co-administered cohort, leading to neuroprotection by clearance of the toxic aggregates. Reduced p-GSK3B signals are observed in DAergic neurons of the 6-Bio treated cohorts, namely, 6-Bio only and MPTP+Co.

Behavioral Studies:

All the behavioural experiments are done on 3-4 month old, male C57/B16 mice. Experimenters are blind to the drug injected animals. Experimenters handle mice used for behavioral experiments for 3 consecutive days prior to the training paradigm. Behavioral experiments are designed in an order of low to high stress activity for mice. Therefore, Open Field Test is conducted in forenoon while rotarod is performed in the afternoon.

Mice were habituated to the behaviour room for 15 minutes every day before start of experiments. The light intensity is maintained at 100 lux throughout the experiment. Mice are weighed every day before training or test to ensure their good health. Mice are randomly allocated into three treatment cohorts: placebo control, MPTP and 6-Bio. Data is plotted using GraphPad prism 5 software.

Rotarod Trials:

The rotarod instrument is custom made at the Mechanical workshop, National Centre of Biological Sciences, Bengaluru, India. The rotating rod (diameter 3.3 cm) is made of Delrin and is textured to enhance the grip of mice. The rod is fixed at a height of 30 cm from the cushioned platform where mice fell on to during training and test. The rod is partitioned into three areas of 9.3 cm distance between each partition using discs (40 cm diameter) made of Teflon. Mice are trained in rotarod for five consecutive days prior to drug injection. Each mouse is trained in rotarod by gradually increasing the rotation on every day. On Day 1, mice are trained on 540 rpm (accelerated 612 at 1 rpm/5 seconds), 11-15 rpm (accelerated at 1 rpm/15 seconds) on second day, 16-20 rpm (accelerated at 1 rpm/5 seconds) on third day and at 20 rpm (fixed) for Day 4 and 5. Mice are trained at above specific rpm for 3 times with 5 minute interval between trials. The rod is rotated from 5 rpm to 20 rpm by manually changing the speed of motor (non-automated). During test (Day 13 post injection), the rotarod is started at 20 rpm. Mice are tested in a rotating rotarod for a maximum of 60 seconds and their latencies were noted down. At the end of each trial, the rotarod is wiped with 70% ethanol and left for drying before placing next set of mice. The entire trial is video recorded using a DSLR camera (Nikon D5100) and latencies are scored manually. The average time spend on rotating rotarod across three trials are plotted as mean latency to fall.

Open Field Test:

Open field arena (50 cm×50 cm×45 cm) is custom-made (JNCASR) using plywood and the internal surface was coated with white polish. Mice are trained in open field for 2 consecutive days prior to drug injection. During training or testing, one animal at a time is placed in zone periphery in open field arena and allowed to explore the arena for 5 minutes. The activity is video recorded (SONY® color video camera, Model no. SSC-G118) using a software (SMART v3.0.04 from Panlah, Harvard Apparatus, USA). After 5 minutes, the mouse is returned to its home cage. The open field arena is then wiped using 70% ethanol and allowed to dry before placing the next mouse. Distance travelled is analyzed offline by an experimenter who was not involved in performing the experiment.

6-Bio Ameliorates MPTP-Induced Behavioral Deficits: 1541

To study whether 6-Bio can combat the MPTP induced behavioral impairments in motor co-ordination, locomotion and exploration abilities, we two widely used behavior tests namely rotarod and Open Field Test are employed. Stereology is performed on day 7 post MPTP/placebo administrations, so behavior experiments are conducted on day 13 i.e., day 7 post MPTP/placebo administrations. In rotarod test, the latency to fall for MPTP cohort reduces significantly to that of placebo cohort on day-13 post-administration (MPTP versus placebo, P<0.001, FIG. 20A) validating the MPTP induced motor deficits in mice. Strikingly, the latency to fall is increased in the 6-Bio treated cohort compared to that of MPTP treated cohort (Co versus MPTP P<0.001, FIG. 20A). The latency to fail for 6-Bio treated cohort is found to be comparable with the placebo cohort (Co day-7 versus placebo, P>0.05, FIG. 20A). Similar to the lack of motor co-ordination observed in rotarod, the total distance travelled by MPTP treated cohort in Open Field Test arena on Day-13 post-injection is reduced significantly compared to that of placebo (MPTP versus placebo, P<0.001, FIG. 20B, FIG. 20C) affirming the MPTP induced locomotion and exploratory impairments.

These impairments are improved after 6-Bio administration as the distance travelled by, mice increased dramatically (Co versus MPTP, P<0.001, FIG. 20B, C) and matched control cohorts (Co versus placebo, P>0.05, FIG. 20B, C and FIGS. 6A and C), MPTP induced impairments are not protected by 6-Bio when administered after 48 h MPTP (Post versus MPTP, P>0.05, FIGS. 20D and E and FIGS. S6B and D).

Since the 6-Bio treated cohort spent more time on the rotarod (as the placebo cohort) and also travelled more distance in open field unlike MPTP treated, it can therefore be can infer that 6-Bio rescued the MPTP induced motor, locomotion and exploratory impairments. It is observed that 6-Bio fails to protect the MPTP induced behavioral deficits when administered 48 h after MPTP dosage.

### Example 5

### Assays for Assessing Action of Small Molecule Inhibitors Bay-11 and ZPCK at Different Stages of Autophagy in Yeast

Standard autophagy assays are performed in S. cerevisiae for the degradation of autophagy markers, such as Pot1-GFP for pexophagy (FIGS. 21A and 21B) and GFP-Atg8 for general autophagy (FIGS. 21E and 21F), Both Bay11 and ZPCK are found to delay the degradation of peroxisomes, as evident from decreased clearance and consequent slow release of free GFP associated with Pot1-GFP (FIGS. 21A-21D) and also slower release of GFP in general autophagy assay using GFP-Atg8 as the marker (FIGS. 3E-3G), which suggests a block in both selective and general autophagy respectively. Fluorescence microscopy studies in S. cerevisiae shows a decrease in the degradation of peroxisomes (labelled with Pot1-GFP) in the presence of both the inhibitors as observed by the accumulation of GFP-positive punctate structures (peroxisomes or pexophagic bodies) inside or outside of the vacuole (labelled with FM4-64) (FIGS. 21H-21L), and lack of diffused GFP signal inside the vacuole (FIGS. 21H and 21K). On the contrary, the number of peroxisomes in the untreated cells decreases in the cytosol with the appearance of diffused GFP inside the vacuole; an indicator of pexophagy (FIGS. 21H, 21K and Vid. S1). However, treatment with 25 μM Bay11 causes the peroxisomes to accumulate outside the vacuoles without any vacuolar diffused GFP pattern even upon starvation (FIGS. 21H, 2111K and Vid. S2). Interestingly, ZPCK (50 μM) treated cells exhibit a build-up of peroxisomes inside the vacuoles (FIGS. 21H, 21L and Vid. S3). Furthermore, fluorescent microscopy of GFP-labelled autophagosomes (GFP-Atg8) shows accumulation of punctate structures outside the vacuole without any free-GFP inside it, on treatment with Bay11 (FIGS. 4A-4C); a morphology similar to an autophagy mutant (Δypt7) that blocks the autophagosome-vacuole fusion step. These results indicate that Bay11 likely perturbs at a step prior to the fusion of autophagosomes with vacuoles, whereas ZPCK inhibits the degradation of autophagic cargo inside the vacuoles.

To elucidate the step of action of Bay11, a protease protection assay is performed using aminopeptidase as a marker, which is also a substrate for starvation-induced autophagy. Untreated cells in presence of proteinase K show both the precursor as well as the matured form due to the autophagosome-sequestered membrane-protected cargo and the cytosolic free form, respectively (FIGS. 22D and 22E). However, Bay11 -treated cells primarily show only the mature form of aminopeptidase upon proteinase K treatment (FIGS. 22D and 22E). Combined treatment with Proteinase K and triton X-100 results in conversion of all the precursor form to the matured form in both treated and untreated groups. Conversion of the precursor to matured form of aminopeptidase in the presence of proteinase K un Bay11 treated cells indicates that the cargo is not protected by the autophagosome membrane, and thus autophagosome biogenesis or maturation or both may be inhibited by Bay11. Co-localization of Atg8 protein with Atg5, a marker for developing autophagosomes, shows only a single Atg5 punctate structure in untreated cells as compared to multiple puncta in Bay11 treated cells (FIGS. 4F and 4G). Also there is significantly more co-localization observed between Atg8 and Atg5 dots in Bay11 treated cells than untreated cells (FIGS. 4F and 4H). This observation taken together with the protease protection assay suggests that the treatment with Bay11 leads to accumulation of incompletely formed autophagosomes.

### Example 6

### Inhibition of Autophagy by Bay 11 and ZPCK in Mammalian Cells

Owing to the conserved nature of autophagy, the putative inhibitors as obtained through the yeast screen are analysed in mammalian cells for their autophagy inhibitory effects.

The effects of Bay11 and ZPCK in mouse cells is assessed for their ability in impairing autophagic cargo degradation by analysing the clearance of the specific autophagy substrate, p62/SQSTM1. In mouse embryonic fibroblasts (MEFs), it is found that both the compounds cause significant accumulation of endogenous p62 aggregates at 24 h and 48 h (FIGS. 5A and 5B).

Bay11 is further analysed with whether this accumulation of p62 is autophagy dependent by employing Atg5+/+ (wild-type) and Atg5−/− (autophagy-deficient) MEFs. As expected, while Bay11 significantly increases endogenous p62 levels in Atg5+/+ MEFs, it has no significant effect in Atg5−/− MEFs (FIGS. 5C and 5D). Likewise, Bay11 reduces MAP1LC3B-II levels in Atg5+/+ MEFs but not in Atg5−/− MEFs that are devoid of autophagosomes or MAP1.LC3B-II (FIG. 23E). Densitometric analyses of MAP1LC3B II/GAPDH and MAP1LC3B-II/MAP1LC3B-I ratios indicate that Bay11 causes a significant reduction of MAP1LC3B-II when analyzed relative to MAP1LC3B-I or GAPDH (FIGS. 5F and 5G). To further dissect the step in the autophagy pathway at which the inhibitors act, an autophagosome maturation assay is performed in HeLa cells (immortalized human cervical cancer cells) using tandem-fluorescent-tagged MAP1LC3B reporter, mRFP-GFP MAP1LC3B.35 This reporter measures the maturation of autophagosomes into autolysosomes, wherein the autophagosomes emit both mRFP and GFP signals (mRFP+/GFP+) whereas the autolysosomes emit only mRFP signal (mRFP+/GFP−) because GFP is acid-labile and is quenched in the acidic environment. Treatment with 2.5 μM Bay11 decreased the number of autophagosomes (mRFP+/GFP+puncta) and autolysosomes (mRFP+/GFP− puncta) in HeLa cells expressing mRFP-GFP-MAP1LC3B, whereas treatment with 25 μM ZPCK increases the number of autolysosomes with no significant change in autophagosomes (FIGS. 24A and 24B). Treatment of the inhibitors along with Bafilomycin Al (BFA), an inhibitor of autophagosome to lysosomal fusion, gave an idea about the inhibition of autophagic flux at different stages.

Bay11 is blocked at a step prior to BFA action, whereas ZPCK acts downstream of BFA (FIGS. 24A and 24C). The MAP1LC3B conversion assay is performed under nutrient rich condition (FIGS. 24D and 24E), starvation condition (FIGS. 24D and 24F) and in the presence of BFA (FIGS. 24D and 24G). Relative changes in MAP1LC3B-II/MAP1LC3B-I and II/TUBB ratios are measured. Bay11 decreases MAP1LC3B-II/MAP1LC3B-I ratio under nutrient-rich and starvation conditions whereas ZPCK increases it in both scenarios (FIG. 24D-24F). However, although ZPCK showed increased MAP1LC3B-II/TUBB ratio, no significant changes are found with Bay11 treatment (FIG. 24D-24F). Nonetheless, analysing autophagosome synthesis with BFA reveals that Bay11 reduces MAP1LC3B12 II/TUBB ratio whereas ZPCK has no significant alterations (FIGS. 24D and 24G). Moreover, a time course experiment with prolonged treatment for 12 hours with Bay11 results in a significant reduction in both MAP1LC3B-II/MAP1LC3B-I (FIGS. 24H and 24I) and MAP1LC3B-II/TUBB (FIGS. 24H and 24J) ratios, further suggesting that Bay11 inhibits autophagosome synthesis. Shorter exposure immunoblots for all the MAP1LC3B conversion assays done for HeLa cells are depicted in FIG. 45. Said data suggests that Bay11 inhibited autophagosome biogenesis whereas ZPCK did not affect this event.

To further assess the impact of Bay11 and ZPCK on autophagic degradation in HeLa cells, the fluorescence intensity of endogenous p62 and its co-localization with mRFP MAP1LC3B-positive autophagosomal compartments is studied. It is observed that p62 accumulates either outside or inside the mRFP-MAP1LC3B-positive compartments upon treatment with Bay11 and ZPCK, respectively (FIGS. 6K and 6L). Moreover, the effects of the autophagy blocker BFA are similar to that of ZPCK. (FIGS. 6K and 6L). This suggests that Bay11 possibly prevents the loading of p62 onto autophagosomes or it can inhibit autophagosome biogenesis, leading to lesser availability for p62 aggregates to co-localize with MAP1LC3B-positive structures.

ZPCK like BFA prevents the degradation of p62 once captured by the autophagosomes, and hence p62 accumulates in MAP1LC3B-positive structures. This result combined with the data using mRFP-GFP-MAP1LC3B reporter (FIG. 24A) suggests that ZPCK inhibits the degradation of autophagic cargo post autophagosome-lysosome fusion.

Next, the effects of Bay11 and ZPCK on other trafficking pathways such as endocytosis using the endocytosis-mediated EGFR degradation assay (FIG. 24M) is assessed Upon EGF treatment, no difference in the degradation of EGFR over time between untreated and treated groups is found, suggesting that the compounds do not affect general endocytic trafficking (FIG. 24N). Since the EGFR degradation is not affected, which normally occurs in the lysosomal compartments, it is likely that lysosomal proteolytic activity is not perturbed.

However, the fact that ZPCK caused accumulation of p62 suggests that it possibly affects some lysosomal protease specific for autophagic cargo.

### Example 7

### Effect of Known and Novel Autophagy Modulators on Lace Plant Aponogeton madagascariensis

Lace Plant Cultures and Experiments

Axenic lace plant cultures are grown in magenta boxes and prepared according to Gunawardena et al. Leaves in the window stage are removed from the corm and rinsed thoroughly with distilled water prior to being sectioned into 2 mm² pieces. For starvation treatments, window stage leaves are removed from the plant, placed in distilled water and kept in the dark overnight.

Leaf sections are stained with monodansylcadaverine (MDC; 300 μM) (Sigma, D4008) and simultaneously treated with autophagy modulators for 2 hours in the dark. (1 hour vacuum infiltration at 15 psi). Treatment times, along with stain and modulator applications are optimized using concentration gradients followed by microscopy. The optimized concentrations are 5 μM rapamycin (Enzo Life Sciences (BML A275-0005), 5 μM wortmannin (Santa Cruz Biotechnology, sc-3505), 1 μM concanamycin A (Santa Cruz Biotechnology, sc-202111), 50 μM Bay 11 (Sigma, B5556) and 50 μM ZPCK (Sigma, 860794).

Tissue sections are then rinsed and mounted in distilled water prior to being scanned using a Nikon Eclipse Ti confocal microscope (Nikon 40X/1.30, Plan Fluor, 405 nm excitation and 450/30 nm emission). Areoles in the early phases of PCD are scanned to avoid cellular debris. The mean number of puncta are quantified for each treatment group with a minimum of four independent experiments using NIS Elements Advanced Research software. Additionally, starvation treatment leaves are also exposed to 5 μM, wortmannin, 50 μM Bay11 and 50 μM ZPCK treatments and then qualitatively assessed via confocal microscopy.

Immunostaining in Lace Plant

ATG8 immunolocalization in lace plant window stage leaves is achieved using a modified protocol from Pasternak et al, 2015.

Whole leaves are treated for two hours prior to fixation in 100% Methanol at 37° C. and then hydrophilized to 20% methanol by adding distilled water at 60° C. every two minutes for 32 minutes. Samples are then sectioned and placed on a multiwall slide and allowed to air dry for 10 minutes to facilitate membrane permeabilization. Blocking is done for 30 minutes at 37° C. with 4% low fat milk powder in 1× microtubule stabilization buffer MTSB (Modified 2× MTSB stock solution: 15 g PIPES, 1.9 g EDTA, 1.22 g MgSO4*7H2O and 2.5 g KOH—pH 7.0). Primary antibody incubation for anti-rabbit ATG8 (Agrisera, AS14 2769) is done at a 1:1000 dilution in 1× MTSB for 30 minutes at 37° C. Samples are then washed for 5 minutes, 3 times with 1× MTSB. Secondary antibody incubation with Goat anti-rabbit Dylight 488 (Agrisera AS09 633) at a 1:2000 dilution in 1× MTSB is done for 30 minutes at 37° C. and then samples are rinsed as above. Tissues are mounted in Mowiol 4-88 solution (Sigma, 81381) and scanned via confocal microscopy as mentioned above. The mean number of puncta per cell is determined using maximum intensity projections (MIPs) for each replicate. The total number of cells within a field of view are counted manually and the number of puncta are counted automatically using ImageJ.

The aquatic lace plant, Aponogeton madagascariensis, has leaves that are nearly transparent and ideal for live-cell imaging. Leaves taken from axenic cultures are sectioned and then assigned to treatment groups. Treatments included a control with no autophagy modulators (FIG. 25A), overnight starvation (FIG. 25A), 5 nM rapamycin (autophagy enhancer),5 μM wortmannin (autophagy inhibitor), 1 μM concanamycin A (autophagy inhibitor) (FIG. 25B), 50 μM Bay11 and 50 μM ZPCK (FIG. 25C) and overnight starvation combined with either 5 μM wortmannin, 50 μM Bay11, or 50 μM ZPCK treatments (FIG. 25E). All leaf sections are stained simultaneously with treatment of the modulators using monodansylcadaverine (MDC). The leaf sections are observed using confocal laser scanning microscopy (CLSM) and the mean number of punctate structures per cell are counted for each treatment group and compared to the control which had a mean of 0.99±0.12 per cell (FIG. 25D). There are significantly fewer puncta in the wortmannin (0.26±0.03) (FIGS. 25B and 25D) and Bay11 (0.28±0.07) (FIGS. 25C and 25D) treatment groups. An overnight starvation for detached leaves resulted in a significant increase in punctate structures (1.90±0.21) (FIGS. 25A, 25D and 25E). A significant accumulation of puncta compared to the control is also observed with the concanamycin A (1.91±0.13) treatment (FIGS. 25B and 25D), but the highest increases in puncta are observed in the rapamycin (2.31±0.25) (FIGS. 25B and 25D) and ZPCK (3.56±0.23) treatment groups (FIGS. 25C and 25D).

In ZPCK treated cells the punctate structures appear to accumulate inside the vacuoles (Vid. S4). Additionally, overnight starvation leaves treated with either Bay11 or wortmannin showed fewer puncta compared to the overnight starvation group, as well as the overnight starvation combined with ZPCK, which have a similar appearance to the starvation group (FIG. 25E). In order to confirm that the puncta observed with MDC staining are autophagosomes, immunolocalization experiments are carried out using an Atg8 antibody from Chlamydomonas which bears 80% resemblance to Arabidobsis Atg8a. The modulators used in MDC experiments are applied to lace plant leaves as mentioned above, the pattern of the punctate structures increases or decreases as expected and is similar to the results obtained from MDC staining procedure (FIG. 26vA-C). The control group (FIG. 26A) has 0.90±0.08 puncta (FIG. 26C) and there is a significant inhibition following wortmannin (0.27±0.025; FIG. 26A) and Bay11 (0.22±0.03; FIG. 26B) treatment. There is a significant increase in puncta following starvation (2.16±0.15; FIG. 26A), rapamycin (3.52±0.09; FIG. 26A), concanamycin A (2.43±0.29; FIG. 26A) and ZPCK (2.09±0.13; FIG. 26B) treatments.

### Example 8

### Assays for Assessing Effect of Acacetin

Pot1-GFP Assay

Pot1-GFP positive strains are allowed to grow in yeast extract peptone dextrose (YPD) (2% dextrose, 2% peptone and 1% yeast extract) till the Absorbance at 600 nm reaches 0.6-0.8. Peroxisome biogenesis is induced by growing these cells in YPG medium (1% yeast extract, 2% peptone, 3% glycerol) for 12 hours.

Cells are harvested, washed twice to remove traces of oleate and transferred to starvation medium with and without Acacetin, at. inoculum density Absorbance at 600 nm 3/ml, to induce pexophagy. Cells are collected at various time intervals after pexophagy induction and processed by TCA precipitation.

Cells treated with Acacetin show an enhanced accumulation of free GFP over time as compared to the untreated cells, which indicates an increase in the levels of pexophagy. (FIG. 11)

Fold Change for Acacetin Using Burden Assay

U1752 cell line and HeLa cell line are infected with Salmonella typhimurium SL1344, and grown overnight in micro-aerophilic condition, at an MOI of 400 for one hour. The cells are treated with media containing Gentamycin at the concentration of 100 μg/ml for 2 hours to kill the extracellular bacteria. The cells are then treated with compounds and incubated for ?hours to 4 hours. At the end, the cells are lysed using lysis buffer (0.1% SDS, 1% Triton X-100, 1× PBS) and the intercellular Salmonella is plated and the CFU is counted. The CFU of Salmonella in the Acacetin treated cells is reduced by almost 2 fold compared to that of the untreated cells. Statistical analysis of the results is done using Graphpad prism—two tailed T test (FIG. 12)

### Example 9

### Effect of Acacetin on Salmonella typhimurium SL1344

A single colony of Salmonella typhimurium WT strain SL1344 grown overnight at 37° C. is diluted in Luria Broth media to get an O.D of 0.2. The diluted culture is used for treatments with Acacetin and Acacetin with gentamycin (100 μg/ml). The growth curve of the culture is obtained by measuring the absorbance at 600 nm using varioskan Flash Multiplate Spectrophotometer at 300 rpm and O.D taken at every 30 minutes interval for 10 hours is plotted using GraphPad Prism.

FIG. 13 illustrates that Acacetin does not have any anti-microbial activity against Salmonella typhimurium SL1344.

### Example 10

### Co-Localization GFP-LC3 with mcherry Salmonella typhimurium SL1344

HeLa cells are transfected with GFP-LC3 using lipofectamine 3000. After 24 hours, cells are infected with Salmonella typhimurium WT strain SL1344 with an MOI of 400 for 15 minutes followed by gentamycin treatment at the concentration of 100 μg/ml for 10 minutes to kill the extracellular bacteria.

The cells are treated with and without Acacetin and incubated for different time points (1, 2, 4 and 6 hours) at 37° C. Quantitation of LC3 co-localization with Salmonella typhimurium SL1344 is done using ImageJ-Cell counter option (FIG. 14), to check whether with compound (Acacetin) treatment more autophagy machinery is recruited towards the bacteria.

### Example 11

### Live Cell Microscopy to Assess Effect of Acacetin on Replication of Salmonella

GFP-LC3 transfected HeLa cells is infected with mcherry-Salmonella typhimurium SL1344 for 15 minutes (MOI-400) and is treated with gentamycin for 10 minutes. The cells are then washed with 1× PBS and changed to either only media (a) and media containing Acacetin (b) and imaged by FV10i-olympus confocal live cell imaging microscope, using 60× water immersion lens, with confocality aperature set to 1.0. Images are taken at an interval of 15 minutes. (c) The intensity of the Red channel denoting the mcherry tagged S. typhimuriumis measured using image J—Stacks T function (intensity of red channel signifies the replication of the S. typhimurium over time). The replication of Salmonella in Acacetin treated samples is restricted as compared to that of untreated samples (FIG. 15)

### Example 12

### Traffic Light Assay for Acacetin

ptf-LC3 transfected HeLa cells are treated with the Acacetin for 2 hours. Following treatment, the number of autophagosomes and autolysosomes are counted using image J-cell counter function. The starvation medium (HBSS), is used as positive control which shows higher counts than the basal level of growth medium (GM). The compound treated sample shows an increase in the number of autolysosomes (red dots) (FIG. 16).

### Example 13

### Assessing the Effect of XCT-790 in Autophagy in Yeast Cells

Yeast media used for culturing is SD-Ura [Synthetic dextrose (2%) medium without uracil] for culturing α-synuclein-EGFP strains (wild type and atg1Δ) and EGFP-Atg8 processing assay, SG-Ura [Synthetic galactose (2%) medium without uracil] to induce α-synuclein-EGFP protein expression. Above mentioned strains are cultured at 250 rpm and 30° C.

Yeast Growth Assays:

Appropriate yeast strains are seeded (A600 ˜0.07) with or without drugs in a 384-well plate and incubated (420 rpm, 30° C. and 80 μl) in a multiplate reader (Varioskan Flash, Thermo Scientific) for 48 h that records absorbance (A600) automatically for every 20 min. Growth curves are plotted using GraphPad Prism.

α-Synuclein-EGFP Aggregates Induction in Yeast:

For inducing α-synuclein-EGFP aggregates in yeast, the appropriate strains are inoculated SD-Ura medium. Then, secondary cultures are inoculated from the primary culture and incubated till A600 reaches 0.8/ml. Cells are washed twice with sterile water and the aggregates are induced by incubating the cultures in SG-Ura for 12-16 h.

α-Synuclein-EGFP Degradation Assays:

After inducing α-synuclein-EGFP aggregates in the corresponding yeast strains driven by galactose promoter, the protein expression is turned off by adding dextrose in the medium. Then, α-synuclein-EGFP aggregates degradation by XCT 790 are assessed by collecting cells treated with and without XCT 790 (50 μM) for 0 and 24 h. Subsequently, the protein levels are analyzed using immunoblotting.

Immunoblot Analysis:

Yeast Lysates Preparation:

The appropriate yeast strains (A600=3) are mixed in trichloroacetic acid (12.5%) and then stored at −80° C. Then, the samples are thawed on ice, centrifuged (16000×g, 15 min) and the pellets are washed twice with ice-cold acetone (80%). Pellets are air dried, resuspended in lysis solution (0.1 N NaOH and 1% SDS) and Laemmli buffer and boiled for 15 min.

Microscopy:

After respective treatments, the yeast cultures were washed, seeded on agarose (2%) pad and then imaged.

A thiadiazoleacrylamide, XCT 790 is found to be one of the ‘Hits’ that showed significant rescue of growth in yeast cells overexpressing α-synuclein (FIG. 39a, FIG. 45). Treating wild-type (WT) yeast cells over expressing α-synuclein with XCT 790 rescued growth lag compared to that of untreated (˜3.2 fold, WT α-syn cells; untreated vs XCT treated, P<0.001, FIG. 39b).

Toxic protein aggregates are known to be substrates of the autophagy pathway for their effective cellular degradation. Consistently, XCT 790 failed to rescue the growth lag in core autophagy mutant cells (atg1Δ) ascertaining its autophagy-mediated rescue of the cells from α-synuclein toxicity (atg1Δ α-syn cells; untreated vs XCT 790 treated, P>0.05, FIG. 39b). Also, in XCT 790 treated atg1Δ cells over expressing α-synuclein cells, the growth related parameters like growth rate (untreated vs XCT 790 treated, P>0.05, FIG. 45f) and doubling time (untreated vs XCT 790 treated, P>0.05, FIG. 45f) are unaltered compared to that of untreated, XCT 790 does not affect the yeast growth at 50 μM (growth rate; untreated vs XCT 790 treated, P>0.05: doubling time; untreated vs XCT 790 treated, P>0.05, FIG. 45b, c). To understand the autophagy modulation by XCT 790 in yeast, GFP-Atg8 (an autophagosome marker) processing assay under both growth and starvation conditions was performed. XCT 790 treatment dramatically induces autophagic flux in nutrient rich growth conditions where autophagy is barely detectable (Autophagy induction; untreated vs XCT treated, P<0.001; Autophagy flux; untreated vs XCT 790 treated, P<0.001, FIG. 39c). Similarly, under starvation conditions XCT 790 significantly induces autophagic flux in a time-dependent manner (untreated vs XCT 790 treated: Autophagy induction: 2h, P<142 0.01; 4 h, P<0.001; 6 h, P<0.001: Autophagy flux; 2 h, P<0.001: 4 h, P<0.001; 6 h, P<0.001, FIG. 46a). In yeast cells overexpressing α-synuclein-EGFP, XCT 790 treatment leads to vacuolar degradation of α-synuclein-EGFP with a restoration of normal plasma membrane localization of α-synuclein-EGFP (˜14 fold, untreated vs XCT 790 treated, P<0.001. FIG. 39d). To validate this, an α-synuclein-EGFP aggregate degradation assay is employed in yeast. Assay scheme is illustrated in FIG. 46B. XCT 790 treatment significantly clears α-synuclein-EGFP in wild-type strain (˜2.5 fold, untreated vs XCT 790 treated, P<0.001, FIG. 39e) but not in autophagy mutant (untreated vs XCT 790 treated, ns, P>0.05, FIG. 39f).

The study, identifies XCT 790 as an autophagy inducer with a potential to clear toxic protein aggregates.

### Example 14

### Assessing the Effect of 6-Bio in Autophagy in Mammalian Cells

SH-SY5Y cells are cultured in DMEM-F12 containing 10% FBS (Life 558 technologies). HeLa cells are cultured in DMEM containing 10% FBS 559 (Pan-Biotech). Cell lines are maintained in following conditions of 37° C. and 5% CO2.

The autophagy assays are performed by seeding equal numbers of sub-confluent 1-HeLa or SH-SY5Y cells in 6-well dishes and allowed to attach for 24 h, then treated with XCT 790 (5 μM) and/or 3-MA (5 mM) and/or lithium chloride (10 mM) in fed condition for 2 h. After treatments, the cell lysates are analyzed by immunoblotting.

RFP-EGFP-LC3 Assay:

Sub-confluent HeLa and/or SH-SY5Y cells are seeded into 60 mm cell culture dishes, then transfected with ptf LC3 construct and/or siRNA and allowed to express for 48 h. Cells are trypsinized, seeded again on poly-D-lysine coated cover slips in a 12 or 24 well plates and allowed to attach. After appropriate treatments, the coverslips containing cells are processed for imaging. For immunofluorescent antibody staining, the cover slips are incubated in primary antibody at 4° C. for overnight followed by secondary antibody incubation at room temperature.

Immunoblot Analysis:

Mammalian cell lysates preparation: After treatments, cells are collected in Laemmli buffer to perform LC3 processing assay, P70S6K, AMPK, ULK1 and 4E-BP1 immunoblotting. Samples are electrophoresed onto SDS-PAGE (8-15%) and then transferred onto PVDF (Bio-Rad) membrane through Transblot turbo (Bio-Rad). Blots are stained with Ponceau S, then probed with appropriate primary antibodies at 4° C. for overnight and subsequently HRP-conjugated secondary antibody. Signals are attained using enhanced chemiluminescence substrate (Clarity, Bio-Rad) and imaged using a gel documentation system (G Box, Syngene) and then bands are quantitated using Image software (NIH).

Microscopy:

For imaging the mammalian cells, after appropriate treatments, coverslips containing cells are fixed using 4% paraformaldehyde (PFA) (Sigma) and then permeabilized using Triton X-100 (0.2%, HiMedia). On slide, coverslips are mounted using antifade, Vectashield mounting medium (Vector laboratories). For antibody staining, coverslips are blocked using 5% BSA for 1 h at room temperature, then incubated with primary antibody at 4° C., overnight and then subsequently probed with corresponding fluorescent dye conjugated secondary antibody.

Images are acquired using DeltaVision Elite widefield microscope (API, GE) with following filters: FITC (490/20 and 529/38), TRITC (542/27 and 594/45) and Cy5 (632/22 and 676/34). Acquired images.

Cell Viability Assay:

SH-SY5Y cells are seeded onto tissue culture treated 96 well plate and then transfected with EGFP-α-synuclein only and/or co-transfected with siRNA. To cells, appropriate drugs are added (24 h) after 48 h of transfection. Using luminescence-based CellTitre-Glo® (Promega) kit, the cell viability is assayed using automated microtitre plate reader Varioskan Flash (Thermo Scientific) are processed using DV SoftWoRX software.

### Example 15

### Mechanism of Actio of XCT 790

XCT 790 Modulates Autophagy through and mTOR Independent Pathway:

Autophagy is regulated by mTOR (mammalian target of rapamycin) dependent and mTOR-independent pathways that are amenable to chemical perturbations 12. To delineate the mechanism of autophagy modulation by XCT 790, the activity of mTOR through monitoring its substrates such as P70S6K and 4EBP1 is examined.

Upon XCT 790 treatment, mTOR activity is unaffected as revealed by its substrates such as phospho-P70S6K and phospho-4EBP1 protein levels which are comparable to that of nutrient rich condition (FIG. 40d and FIG. 48b). In contrast, the levels of phospho-p70S6K and phospho-4EBP1 are attenuated under starvation conditions where autophagy is regulated in an mTOR-dependent manner. Lithium Chloride (10 mM), known to induce autophagy through an mTOR independent mechanism serves as positive control (FIG. 40d, FIG. 48b). These observations assert that XCT 790 is an mTOR independent autophagy modulator.

It is further examined whether XCT 790 exerts its effects through AMPK pathway, one of the predominant mTOR-independent mechanisms known to regulate autophagy. It is observed that treatment of XCT 790 for 2 hours did not affect the activity of AMPK, as evident by the unchanged T172 phosphorylation of AMPK (FIG. 40e) when compared to nutrient rich conditions. AMPK promotes autophagy in an mTOR-independent manner by directly activating Ulk1 through phosphorylation of Ser 555.

Whereas, under nutrient sufficiency, high mTOR activity inhibits Ulk1 activation by phosphorylating Ulk1 at Ser 757 and disrupting the interaction between Ulk1 and AMPK.

Therefore, it is further examined the regulation of levels of activating (S555) and inhibitory (S757) phosphorylation of ULK1 by XCT 790. Consistent with unchanged levels of phosphorylated AMPK after treatment with XCT 790 for 2 hours, the downstream phosphorylation of ULK1 at S555 is unaffected and comparable to the nutrient rich conditions (FIG. 40e). This suggests that XCT 790 does not exert its effects through AMPK pathway, importantly, mTOR-dependent phosphorylation of ULK1 at S757 remains unaltered in XCT 790-treated cells unlike in starvation conditions, where a concomitant decrease in the phospho-ULK1 S757 protein levels is observed. These results further confirm that XCT 790 acts through an mTOR-independent mechanism but not through AMPK pathway.

XCT 790 Induces Autophagy through Regulation of estrogen-Related Receptor Alpha (ERRα):

XCT 790 is found to be the first potent and selective inverse agonist of ERRα 9. To elucidate the role of ERRα in contributing to the function of XCT 790 as autophagy inducer, the following two approaches are used:

a) siRNA-based silencing of ERRα,

b) over expression of ERRα

To evaluate the level at which the knockdown exerts its effects, cells are transfected with siRNAs targeting ERRα. A non-targeting pool is used as a control. Post 48 hours of transfection, the effect of knockdown on regulation of autophagy by ERRα is monitored by microscopy based tandem RFP-EGFP-LC3 assays.

Knockdown efficiency is confirmed by western blotting to be around 80% (Scrambled vs ERRα siRNA, P<0.001, FIG. 41a). Consistent with the effect of XCT 790 knockdown of ERRα also results in a significant induction of autophagosomes (˜5 fold, Scrambled vs ERRα siRNA treated, P<0.001, FIG. 41b) and autolysosomes (˜3 fold, Scrambled vs ERRα siRNA treated, P<0.001, FIG. 41b).

Autophagosome and autolysosome numbers in XCT 790 treated and ERRα downregulated cells are found to be comparable. These results suggest that XCT 790 modulated autophagy through ERRα.

This question is addressed through another approach to understand the autophagy modulation upon overexpression of ERRα. In ERRα over expressed cells, more autophagosomes (˜2 fold, P<0.01, ERRα overexpressed vs untreated) and less autolysosomes (˜2 fold, P<258 0.01, ERRα overexpressed vs untreated) are found than that of control (FIG. 41d, e). From this, it can be interpreted that autophagy is inhibited at its autophagosome to lysosome fusion step upon over expression of ERRα.

When XCT 790 is treated in ERRα over expressed cells, more autophagosomes (˜2 fold, P<0.01, ERRα overexpressed vs untreated) and less autolysosomes (˜2 fold, P<0.01, overexpressed vs untreated) than that of untreated are found (FIG. 41d, e). This autophagic scenario is similar to that of only ERRα over expressed cells (Autophagosomes; ERRα over expressed +XCT 790 vs ERRα overexpressed only, ns, P>0.05, FIG. 41d, e). When ERRα is over expressed, the autophagic modulating ability of XCT 790 is indeed abrogated.

Collectively, these results suggest that XCT 790 modulates autophagy through ERRα.

ERRα Regulates Autophagy by Localizing onto Autophagosomes:

From knock down and over expression of ERRα studies, there is a clear indication that ERRα can modulate autophagy pathway. Autophagy is induced when ERRα is downregulated (FIG. 41b,c) but inhibited when over expressed (FIG. 41d, e).

It is examined whether active transcription is required for autophagic function of XCT 790. Upon XCT 790 treatment in presence of actinomycin D, the autophagosomes and autolysosomes are similar to that of only XCT 790 (XCT 790+Act D vs XCT 790 only, P>0.05. FIG. 49a). This result indicates that autophagic activity of XCT 790 remains unaffected when active transcription is inhibited.

It is then attempted to assess whether ERRα localizes to autophagic related structures such as autophagosomes and autolysosomes. It is analysed if ERRα localizes with either autophagosomes autolysosomes. FCC of ERRα with autophagosomes (˜0.85) are found to be significantly more than that with autolysosomes (˜0.3) under nutrient rich condition (˜2.5 fold, autophagosomes vs autolysosomes, P>0.001, FIG. 42a, c). In basal autophagy conditions, colocalization of ERRα with autophagosomes is significantly reduced in ERRα silenced and XCT 790 treated cells (˜3.5 fold, untreated or scrambled siRNA vs ERRα siRNA, P<0.001, FIG. 42a, c). Significantly more ERRα colocalize with autophagosomes when ERRα is over expressed (control or scrambled siRNA vs ERRα over expressed, P<295 0.001, FIG. 42a, c).

Colocalization of ERRα with autolysosomes is not regulated when compared to that of control (control or scrambled siRNA vs ERRα siRNA or XCT 790 or ERRα over expressed, P>0.05, FIG. 42a, c) suggesting that ERRα might not interact with the autolysosomes. ERRα could localize most likely to autophagosomes than autolysosomes. ERRα might regulate autophagy through its non-canonical LIR motif. These results show that ERRα might regulate autophagy through its localization with the autophagosomes.

### Example 16

### Studies on MPTP Mouse PD Model for XCT-790

Animal studies: All procedures in this study are approved by JNCASR Institute Animal Ethical Committee and conducted as per institutional guidelines. Inbred male C57BL/6 mice (3-4 months old) were used for all experimental groups (n=6). The animals are maintained under standard laboratory conditions i.e. temperature 25°±2° C., 12 h light: 12 h dark cycle and 50±5% relative humidity with ad libitum access to food and water.

MPTP.HCl and XCT 790 Treatment:

The mice are distributed into three groups' viz., vehicle, MPTP and MPTP+XCT 790 injected respectively. The vehicle group receives intraperitoneal injections of dimethyl sulfoxide (DMSO) injections i.e. the solvent. The MPTP group receives 23.4 mg/kg MPTP.HCl in 10 ml/kg body wt. of saline is administered intraperitoneally for 4 times at 2 h interval. The MPTP+XCT 790 group mice are injected with 5 mg/kg body wt, of XCT 790 dissolved in DMSO, alongside the first MPTP injection. The treatment is continued by administering XCT 790 in “an injection a day regime” for 6 days. All the mice are sacrificed 7 days after MPTP administration and the brains are processed for immunohistochemistry.

Tissue Processing for Immunohistochemistry:

The mice are anaesthetized using halothane inhalation and perfused intracardially with saline, followed by 4% buffered paraformaldehyde (pH 7.4), The brains are removed quickly and post fixed in the same buffer tier 24-48 h at 4° C. and cryoprotected in increasing grades of sucrose. Coronal midbrain cryosections of 40 μm thick are collected serially on gelatinized slides. Every sixth midbrain section is used for immunostaining.

Immunoperoxidase Staining of Tyrosine Hydroxylase (TH):

Briefly, the endogenous expression of peroxidase is quenched using 0.1% H2O2 in 70% methanol, followed by blocking of non-specific staining by 3% buffered solution of bovine serum albumin for 4 h at room temperature. The sections are then incubated with the rabbit polyclonal anti-TH antibody (1:800, Santacruz Biotechnology Inc, USA), followed by anti-rabbit secondary antibody (1:200 dilution; Vector Laboratories, Burlingame, USA). The tertiary labelling is performed using avidin-biotin complex solution (1:100, Elite ABC kits; Vector Laboratories, USA).

The staining is visualized using 0.05% solution of 3′-3′-diaminobenzidine, in 0.1 M acetate imidazole buffer (pH 7.4) with 0.1% H2O2. Phosphate buffered saline (0.01 M) containing 0.3% Triton X-100 (0.01M PBST, pH 7.4) is used as both diluent and washing buffer. Appropriate negative controls are processed identically.

Stereological Quantification of TH-Immunoreactive (TH-ir) Neurons at SNpc:

Stereological quantification of TH-ir dopaminergic neurons is performed using optical fractionator probe. The SNpc is delineated on every sixth. TH-ir midbrain section using 4× objective of the Olympus BX61 Microscope (Olympus Microscopes, Japan) equipped with StereoInvestigator (Software Version 7.2, Micro-brightfield Inc., 664 Colchester, USA). The cells are counted using oil immersion lens 665 (100×), with a regular grid interval of 22500 μm² (x=150 μm, y=150 μm) and counting frame of 3600 μm² (x=60 μm, y=60 μm).

The mounted thickness averages to 25 μm. A guard zone of 4 μm is implied on either side, thus providing 17 μm of z-dimension to the optical dissector. The quantification is performed starting with the first anterior appearance of TH-ir neurons in SNpc to the caudal most part in both hemispheres and added to arrive at the total number. The volume of SNpc is estimated by planimetry.

Densitometry Based Image Analysis:

The offline evaluation of TH expression is performed on high magnification images of TH immunostained nigral dopaminergic neurons using Q Win V3 (Leica Systems, Germany); a ‘Windows’ based image analysis system. A cumulative mean is derived from the values obtained from sampling approximately 200 dopaminergic neurons per animal, and expressed as grey values on a scale of 0-255, where ‘0’ means absence of staining and ‘255’ equals intense staining.

Immunofluorescence Based Double Staining of SNpc Dopaminergic Neurons:

The sequential immunolabeling procedure is used to co-label the TH and LC3 and/or A11. First, the midbrain sections are equilibrated with 0.1 M PBS (pH 7.4) for 10 min and then incubated with buffered bovine serum albumin (3%) for 4 h to block non-specific epitopes. Then, the sections are incubated in rabbit anti-LC3 antibody (1:1000) and/or anti-oligomer antibody (A11, 1:1000) for 72 h at 4° C. After subsequent washes, the sections are incubated in corresponding fluorescent secondary antibody (1:200) at 4° C., overnight.

Co-labeling with TH is performed on the same sections using rabbit anti-TH antibody (1:500), followed by secondary labeling. PBST (0.01 M, pH 7.4) is used as both working and washing buffer. Sections are then mounted using Vectashield hardset mounting medium.

Considering that the autophagic mechanism is highly conserved between the yeast and the mammalian system, the potential of XCT 790 to clear toxic α-synuclein protein aggregates through autophagy in mammalian cells such as human neuroblastoma SH-SY5Y and HeLa cell lines is validated.

To test the modulation of mammalian autophagy and its flux by XCT 790, western blot analysis based LC3 (autophagosome marker) and microscopy-based tandem RFP-EGFP-LC3 assays are employed. In tandem RFP-EGFP-LC3 assay, XCT 790 treatment significantly induces autophagosomes and autolysosome formation in both SH-SY5Y (control vs XCT 790 treated, autophagosomes, ˜2 fold, P<0.05; autolysosomes, ˜4 fold, P<0.01, FIG. 48a) and HeLa cells (control vs XCT 790 treated, autophagosomes, ˜5 fold, P<0.001; autolysosomes, ˜2 fold, P<0.001, FIG. 40a). Additionally, XCT 790 treatment enhances accumulation of LC3-II levels indicating the induction of autophagy (˜2.5 fold, untreated vs XCT 790, P<0.001, FIG. 40b).

These results demonstrate that XCT 790 also modulates mammalian autophagy as in yeast.

The question whether XCT 790 protects SH-SY5Y cells from EGFP-α-synuclein mediated toxicity is then addresses. Overexpression of EGFP-α-synuclein SH-SY5Y cells is toxic and leads to its significant cell death as measured by cell viability assay (˜4 fold, vector control or untransfected vs a-syn transfected, FIG. 40c). Upon administration of XCT 790 to cells overexpressing EGFP-α-synuclein, the cell viability increases significantly than that of untreated (˜4 fold, α-syn over expressed cells, untreated vs XCT 790 treated, P<0.001, FIG. 40c) and comparable to that of vector control (vector control vs α-syn over expressed cells XCT 790 treated, ns, P>0.05, FIG. 40c).

It is observed that potential of XCT 790 to protect from EGFP-α-synuclein toxicity is abrogated in presence of pharmacological autophagy inhibitor, 3-MA (α-syn over expressed cells, XCT 790 vs XCT 790+3-MA, ˜4 fold, P<0.001, FIG. 40c) that is comparable to that of XCT 790 untreated (α-syn over expressed cells, untreated vs XCT 790+3-MA, ns, P>0.05, FIG. 40c). These results clearly demonstrate that XCT 790 protects human neuroblastoma cells from EGFP-α-synuclein mediated toxicity in an autophagy dependent manner. It is demonstrated that XCT 790 exerts protection to the cells against EGFP-α-synuclein mediated toxicity by inducing autophagy which helps clear the toxic aggregates.

XCT 790 Alleviates the MPTP Induced Dopaminergic Neuronal Loss:

A significant proportion of dopaminergic neurons in Substantia Nigra pars compacta (SNpc) are lost after MPTP treatment (˜68%, MPTP vs Vehicle, P<0.001, FIG. 43a,b) as previously described. Co-administration of XCT 790 with MPTP, however alleviated this loss by 80% (MPTP+Co vs Vehicle, P<0.05; XCT 790 vs MPTP, P<0.01, FIG. 43a,b). In a congruent manner, volume of SNpc reduces significantly after MPTP injection (MPTP vs Vehicle, P<0.01, FIG. 50c), whereas the shrinkage is prevented by approximately 85% when MPTP and XCT 790 are administered together (MPTP+Co vs MPTP, P<0.01, FIG. 43b, FIG. 50c).

Cellular Tyrosin Hydroxylase (TH) Expression is preserved in XCT 790 Co-Treatment Group: The cellular TH expression of individual TH-immunoreactive (TH-ir) dopaminergic, as measured by densitometry, is significantly reduced in surviving neurons in MPTP group (MPTP vs Vehicle, P<0.001, FIG. 49b). TH expression in the nigral neurons of MPTP and XCT 790 co-treated mice is comparable to that of the vehicle control group. Thus, XCT 790 significantly alleviates the MPTP induced depletion of cytoplasmic TH expression (MPTP+Co vs MPTP, P<0.001, FIG. 49b).

XCT 790 Enhances Autophagy and Clears Toxic Protein Aggregates in an In-Vivo Mouse Model of PD:

In neurons, the autophagy process is indispensable for clearing the misfolded toxic protein aggregates. During the neurodegenerative progression, autophagy would be defunct and becomes incompetent to maintain cellular proteostasis.

To delineate the mechanism of neuroprotective action of XCT 790, the autophagy status is examined in the various mice treatment cohorts. Yeast and cell lines results strongly indicate that XCT 790 might exert neuroprotection through modulating autophagy. In MPTP toxicity model, the LC3 puncta per neuron is reduced significantly than that of vehicle treated (˜0.8 fold, vehicle vs MPTP treated, P<0.01, FIG. 43c,d) indicating the dysfunctional autophagy during neurodegenerative disease progression.

Interestingly, XCT 790 only cohort exhibits significantly increased LC3 puncta per cell compared to that of vehicle treated cohort (˜3 fold, vehicle vs XCT 790 only, P<0.001, FIG. 43c,d). This demonstrated that XCT 790 is a strong autophagy inducer in the dopaminergic neurons of SNpc. A significantly increased LC 3 puncta per cell is observed in the MPTP and XCT 790 co-administered than that of vehicle treated cohort (˜3 fold, vehicle vs MPTP+Co, P<0.001, FIG. 43c,d). These results demonstrate that XCT 790 could induce autophagy in the brain and strikingly surpass the autophagic deficit caused due to pathogenesis.

During protein aggregation, the toxic misfolded protein oligomeric species would be accumulated in the neurons. It is examined whether autophagy induction by XCT 790 could clear the toxic oligomeric intermediates in the neurons. In a vehicle treated cohort, the occurrences of aggregates are significantly less compared to that of MPTP treated cohort (˜6.5 fold, vehicle vs MPTP treated, P<0.001, FIG. 43e, f). These observations reaffirm that toxic misfolded protein aggregates are formed during disease pathology. Upon co-administration of MPTP along with XCT 790, it is observed that there is a significant reduction in the toxic aggregates compared to that of MPTP only treated cohort (˜6 fold, MPTP vs MPTP+Co, P<0.001, FIG. 43e, f). It is found that aggregate reduction in the MPTP+Co cohort is comparable to that of vehicle treated cohort (MPTP+Co vs vehicle, ns, P>0.05, FIG. 43e, f) indicating its strong potential to clear misfolded toxic protein aggregates.

In addition, the presence of aggregates in the steady state level of cell in the XCT 790 only is comparable to that of vehicle cohort (vehicle vs XCT 790 only, ns, P>0.05, FIG. 43e, f). This result indicates that administrated dosage regimen is not exerting any proteotoxic stress to the neurons. It is demonstrated that XCT 790 could clear the pathological toxic misfolded protein aggregates upon disease progression, one of the main causative of neurodegeneration.

Mechanistically XCT 790 exerts neuroprotection by clearing misfolded protein aggregates through inducing autophagy demonstrated in the in-vivo preclinical mouse model of PD.

XCT 790 Ameliorated MPTP-Induced Behavioral Impairments:

Parkinson's disease patients exert movement disorder symptoms such as motor co-ordination, exploration and locomotion disabilities that can be recapitulated in a MPTP mice toxicity model.

As data herein shows neuroprotective role of XCT 790 at both cellular and tissue level, it is tested whether its effect can be translated up to behavioral level. To address this, a set of well-known behavioral experiments is performe-Rotarod and Open Field tests—specific for assaying the movement disorders.

To test the exploratory ability of mice, the distance travelled in periphery zone of open field arena is compared across different cohorts. It is observed that distance travelled in the zone periphery is drastically reduced in MPTP treated cohort compared to that of vehicle treated cohort (MPTP versus vehicle control, P<0.001, FIG. 44d,e) on both day 13 and day 15, validating the MPTP's effect on exploratory ability.

Upon co-administration of XCT 790 along with MPTP, the distance travelled is significantly more than that of MPTP cohort (Co versus MPTP cohort, P<0.001, FIG. 44d, e), and more importantly comparable to that of vehicle treated cohort on both day 13 and day 15 (Co versus vehicle control, P>0.05, FIG. 46d,e).

Importantly, exploratory behaviour of various cohorts is evident in the trajectory maps (FIG. 44c). Rotarod test, another standard behavioral assay to test motor coordination is also employed. In this test, the time spent by mice on a horizontal rotating rod (latency to fall) is used to assay the motor coordination ability across different cohorts. In parallel to the results observed in case of Open Field, the co-treated cohort shows improved latency to fall compared to that of MPTP treated cohort (Co versus MPTP cohort, P<0.001, FIG. 44a, b) which shows reduced latency to fall against vehicle treated cohort on both day 13 and day 15 (MPTP versus vehicle control, P<0.001, FIG. 44a,b).

Also, the results of vehicle treated and co-treated cohorts are fairly comparable (Co versus vehicle control, P>0.05, 44a, b) on both days. Therefore, these results suggest restoration of exploratory and motor coordination abilities in MPTP toxicity model, upon administration of XCT 790. Therefore, XCT 790 ameliorates the behavioural disabilities of MPTP treated mouse model.

1-22. (canceled)

23. A method of modulating autophagy in a cell comprising step of—
i) activating autophagy by contacting the cell with compound selected from a group comprising 6-Bio XCT-790 and acacetin or a combination thereof, wherein during activation, the 6-Bio and the XCT-790 enhance fusion of autophagosome and lysosome and the acacetin induces formation of autolysosome in the cell infected with microorganism, wherein the acacetin causes degradation of the microorganism in the infected cell; or,
ii) inhibiting autophagy by contacting the cell with Bay-11 ZPCK or a combination thereof, wherein during inhibition, the Bay-11 inhibits autophagosome lysosome fusion, autophagosome biogenesis or autophagosome maturation and the ZPCK inhibits degradation of autophagic cargo inside the vacuole after fusion of autophagosome and lysosome;
during activation, the 6-Bio is mTOR dependent and the XCT-790 is mTOR independent.

24. The method as claimed in claim 23, wherein the 6-Bio and the XCT-790 cause degradation of α-synuclein (SNCA).

25. The method as claimed in claim 23, wherein the 6-Bio enhances fusion of autophagosome and lysosome in the cell by about 8 fold to 10 fold.

26. The method as claimed in claim 23, wherein the 6-Bio modulates autophagy by passive diffusion and the 6-Bio is GSK3B dependent.

27. The method as claimed in claim 23, wherein the microorganism is selected from a group comprising Salmonella typhimurium Legionella pneumophila, Listeria monocytogenes, Shigella flexneri, Streptococcus pyrogenes, Mycobacterium tuberculosis, or any combination thereof.

28. The method as claimed in claim 23, wherein the autophagy is selected from a group comprising macroautophagy, chaperone mediated autophagy, microautophagy, mitophagy, pexophagy, liphophagy, reticulophagy, ribophagy, zymophagy, Aggrephagy, xenophagy, or any combinations thereof.

29. The method as claimed in claim 23, wherein the cell is eukaryotic cell selected from a group comprising yeast cell, plant cell and mammalian cell, or a combination thereof.

30. The method as claimed in claim 23, wherein the concentration of the 6-Bio, XCT-790 Acacetin, Bay-11 and ZPCK is ranging from about 1 μM to about 150 μM.

31. A modulator of autophagy selected from a group comprising 6-Bio, XCT-790, Acacetin, Bay-11 and ZPCK or any combination thereof for enhancing formation of autolysosome by promoting autophagosome and lysosome fusion, or inhibiting autophagosome biogenesis, autophagosome maturation or degradation of autophagic cargo following autophagosome-lysosome fusion, or any combination thereof, thereby increasing or decreasing autophagic flux.

32. The modulator of autophagy as claimed in claim 31, wherein the 6-Bio and the XCT-790 enhance autolysosome formation in the cell and cause degradation of α-synuclein (SNCA).

33. The modulator of autophagy as claimed in claim 32, wherein the 6-Bio modulates autophagy by passive diffusion and wherein the 6-Bio is mTOR dependent and GSK3B dependent; wherein the XCT-790 is mTOR independent while modulating the autophagy and wherein the XCT-790 is inverse agonist of ERRα.
